(19) United States
(12) Patent Application Publication
(10) Pub. No.: US 2026/0283924 A1
(43) Pub. Date: Sep. 24, 2026

(54) TOPICAL COMPOSITIONS FOR PROMOTING HAIR GROWTH

(71) Applicant: ESTETRA SRL, Liège (BE)

(72) Inventors: Céline GERARD, Liège (BE); Valérie DION, Liège (BE)

(73) Assignee: ESTETRA SRL, Liège (BE)

(21) Appl. No.: 19/474,382

(22) PCT Filed: Apr. 8, 2024
(86) PCT No.: PCT/EP2024/059517

§ 371 Date: Oct. 10, 2025

(30) Foreign Application Priority Data
Apr. 12, 2023 (EP) 23167584.4

(51) Int. Cl. A61K 8/63; A61Q 5/00; A61Q 7/00

(52) U.S. Cl. CPC A61K 8/63; A61Q 5/00; A61Q 7/00

(57) ABSTRACT

The present invention relates to topical compositions for preventing female or male hair loss and/or promoting hair growth, a cosmetic or therapeutic treatment wherein such compositions are used and to the associated formulations or dosage units comprising such compositions.

### FIELD OF THE INVENTION

The present invention relates to topical compositions for preventing hair loss and/or promoting hair growth, a cosmetic or therapeutic treatment wherein such compositions are used and to the associated formulations or dosage units comprising such compositions.

### BACKGROUND ART

With aging, the rate of hair growth declines accompanied by a reduction in hair thickness. Grey hairs appear as the result of decreased melanin production within the hair matrix. In general, the hair follicle cycle is characterized by a period of follicle growth (anagen), followed by a period of regression and remodelling (catagen), and finally by a resting period (telogen).

Estrogen levels have been related to both hair growth and hair loss. For example, during pregnancy a woman's estrogen levels are higher than normal, which increases hair follicle growth and leading to full, thick hair. In contrast, when the estrogen level decreases such as after a pregnancy or during and after menopause, more hair follicles will enter into a “resting” phase, leading to hair loss causing thinning and even balding patches. The change in hormone balance can have an influence on the skin composition and on the functioning of the hair follicles. Also, the secretions of the sebaceous glands (sebum) provides emollients for the hair and skin and are under the influence of androgen hormones. Sebum secretion declines with age and can be influenced by estrogen levels. These effects are aggravated in menopausal women (i.e. perimenopausal and postmenopausal woman) due to the effects of hormonal imbalance, often leading to a negative self-image.

In men, hair loss is a common phenomenon, and numerous different forms and causes having been described in the art. For example, androgenetic alopecia (AGA) in men is characterized by follicular miniaturization in a patterned hair loss occurring due to systemic androgen and genetic factors. The prevalence and severity of AGA is considered to be ethnic group-specific, with Caucasian men having a higher prevalence (up to 50% of the population) when compared to Asian and black men (Salman et al., An Bras Dermatol, 2017). The onset typically coincides with the end of puberty and continues progressively throughout life. In some cases male hair loss may impact body image satisfaction and self-esteem.

In view of the above, intensive research was conducted the past decades in order to prevent, reduce, or even reverse both female and male hair loss. Several medicinal (e.g. minoxidil, finasteride) and chirurgical treatment strategies (e.g. transplantation) enjoy widespread use. Both systemic dosage units and topical compositions are available for treating female or male hair loss, each approach having their own set of advantages and disadvantages. However, the applicability and/or efficacy remains highly patient-specific and therefore any additional treatments remain of high value to the field.

Hormonal imbalances have been reported to impact hair loss in both women and men (Kische et al., JAMA Dermatol, 2017). The potential impact that estrogens have on female hair growth have been described in the art (Brough and Torgerson, Int J Womens Dermatol, 2017). In addition, the potential impact that estrogens may have on male hair loss has been studied extensively and this research has led to the conclusion that an increased estrogen level may contribute to male balding (Hu et al., PLoS One, 2012). In this context, reference is for example made to PCT application WO03103685 which reports on the topical application of a cosmetic composition comprising estetrol. Notably, the document suggests that some of the effects of applying estrogen on the skin include slowing down of the rate of hair growth, which was seen as a positive thing to avoid e.g. facial hair growth in menopausal women.

In general, topical compositions have been traditionally preferred by users. In addition, regardless of the active pharmaceutically ingredient(s), topical application generally results in a reduced risk of systemic (adverse) effects. Moreover, topical compositions allow to conveniently combine several active ingredients within a composition, such that enhanced personal care products can be formulated (e.g. shampoo for reducing hair loss).

Topical compositions and formulations comprising an estrogen have been described in the art. An example of a commercially available topical formulation is EstroGel®, which is an estradiol-comprising gel. EstroGel® is indicated in the treatment of moderate to severe vasomotor symptoms and moderate to severe symptoms of vulvar and vaginal atrophy due to menopause. Although potential for wound healing indications is assumed, the use is not pursued because of undesirable systemic side effects. Indeed, it is known that conventional estrogen formulations are suboptimal for direct application to individuals and are characterised by a considerable list of adverse effects that may be linked to the use of estradiol and/or unintended systemic exposure. Such adverse effects can include nausea, vomiting, stomach cramps, bloating, swelling, weight gain, breast pain, breast tenderness, headache, vaginal itching, vaginal discharge, aberrant menstrual regulation, spotting, hair loss, etc. (cf. Mayo Clinic report on transdermal estradiol: Estradiol (Transdermal Route) Side Effects—Mayo Clinic). Hence, known pharmaceutical compositions are accompanied by considerable amounts of unwanted systemic effects on the individual.

It is thus evident that there is an ongoing need for new topical formulations that are effective in reducing, stopping, or even reversing hair loss in both female and male. The value of such formulations is considered to extend beyond the mere aspect of hair loss reduction but may improve the psychological self-image of both women and men.

### SUMMARY OF THE INVENTION

The hair follicle (HF)'s capability to constantly self-renew and undergo repeated cycles relies on the reciprocal interactions that occur between its mesenchymal and epithelial compartments, so-called epithelial-mesenchymal interactions (EMIs). Therefore, successful strategies aiming to promote HF regeneration/stimulation will work best when targeting the combination of relevant cell populations as well as their functioning/signalling and/or migration. The HF mesenchyme is mainly represented by specialized dermal papilla (DP) cells, whereas the epithelial fraction is commonly comprising keratinocytes (KCs) which can be present in different follicular sources, including the bulge, the outer root sheath (ORS) or the hair bulb itself. Such epithelial cells can be derived from epidermal KCs with stem-like features, either residing in the hair follicle itself or present in the skin and that are capable of forming HF epithelium and create EMIs with DP cells, support DP cell growth, promoting HF formation and of sebaceous gland-like structures (Abreu et al., 2021, Stem Cell Research & Therapy volume 12, Article number: 62).

In postnatal life the hair follicles undergo cyclical growth, comprising a resting phase (telogen), a growth phase (anagen) and a regression phase (catagen). During catagen, the epithelial (keratinocytes) cells at the base of the follicle undergo apoptosis, but the DP cells (fibroblast) remain intact and are pulled or migrate upwards, until they come to rest next to the stem cells of the hair follicle bulge. This situation continues in the telogen (resting) phase. In anagen, the cells at the base of the hair follicle start to proliferate, which results in a downward growth of the follicle and envelopment of the DP. Although DP cells themselves are believed to (virtually) not divide, the number of cells in the DP increases during anagen, possibly as a result of replenishment from neighboring cells of the dermal sheath (mesenchymal cells—fibroblast). At the onset of anagen (growth) the DP activates stem cells in the secondary hair germ leading to new downward growth of follicles. During catagen (regression), the hair follicle detaches from the blood supply and follicle cells undergo apoptosis.

Against this background and in contrast to earlier reports in the art, the present inventors have now surprisingly found that topical administration of estetrol in a specific dosage range can be beneficial for preventing, inhibiting, or even reversing hair loss. Topical treatment of the scalp leads to an improvement in hair texture, hair quality and hair appearance. More particularly, the treatment leads to an improvement in certain parameters such as but not limited to target area hair count (“TAHC”) and target area hair width (“TAHW”).

Estetrol, unlike estradiol, can act both on epidermal keratinocytes including e.g. bulge, the outer root sheath (ORS) or the hair bulb cells, on dermal fibroblasts such as e.g. dermal papilla (DP) cells and on the sebaceous gland. Estetrol is shown to have several effects that are beneficial for preventing or reducing hair loss, ranging from influencing fibroblast and keratinocyte growth, migration and epithelial-mesenchymal interaction, over increasing follicle activation and sebum secretion. In addition, estetrol has been shown to have an influence on reducing inflammation of the skin, which is linked to healthiness of hair follicles. These characteristics render estetrol particularly interesting for inclusion in topical compositions. Moreover, topical administration of estetrol was found to be safe, with no or limited effect on key safety parameters (neither locally at the site of administration nor at the systemic level).

Without wanting to be bound by any theory, it is believed that estetrol (unlike e.g. estradiol) by acting on keratinocytes may promote and/or prolong anagen and hence growth of the hair follicles and/or may delay the onset of catagen by increasing proliferation of keratinocytes and/or reducing apoptosis thereof, thereby reducing hair loss. In addition, by acting also on fibroblasts (i.e. including DP cells), the epithelial-mesenchymal interaction is believed to be improved by estetrol, leading to active DP cells and hair growth.

This finding is directly applicable to any kind of topical composition such as but not limited to therapeutic topical compositions, prophylactic topical compositions, and cosmetic topical compositions. In the following numbered aspects, some particular embodiments of the invention are described:

Aspect 1. A composition comprising an effective amount of an estetrol component for topical use in preventing or treating hair loss in a subject, or for topical application, such as for topical application to the scalp. Thus, the invention is directed to a topical composition for use in preventing or treating hair loss in a subject.

Aspect 2. The topical composition for use according to aspect 1, wherein the subject is a female or male subject. Optionally, the subject is a female subject. Alternatively, the subject is a male subject.

Aspect 3. The topical composition for use according to aspect 1 or 2, wherein the subject is a male subject from 18 to 49 years old.

Aspect 4. The topical composition for use according to any one of the preceding aspects, wherein said composition comprises of from about 0.01% to about 5% (w/w) of an estetrol component, preferably of from about 0.02% to about 1% (w/w) of an estetrol component, more preferably of from about 0.03% to about 0.75% (w/w) of an estetrol component, yet more preferably of from about 0.04% to about 0.5% (w/w) of an estetrol component, most preferably about 0.06% (w/w) of an estetrol component. Preferably, said topical composition is configured and/or formulated for topical application to the scalp.

Aspect 5. The topical composition for use according to any one of the preceding aspects, wherein said composition comprises of from 0.02% to 1.5% (w/w) of an estetrol component, preferably comprising of from 0.05% to 1.2% (w/w) of an estetrol component, of from about 0.02% to about 1% (w/w) of an estetrol component of from about 0.03% to about 1% (w/w) of an estetrol component, preferably of from about 0.04% to about 1% (w/w) of an estetrol component, more preferably of from about 0.05% to about 1% (w/w) of an estetrol component, most preferably from about 0.06% to about 0.5% (w/w) of an estetrol component, more preferably of from about 0.09% to about 1.1% (w/w), even more from 0.1% to 1% (w/w) of an estetrol component, most preferably of from 0.3% to 0.7% (w/w) of an estetrol component.

Aspect 6. The topical composition for use according to any one of the preceding aspects, wherein said composition does not result in a significant systemic effect in the subject upon or after topical application. While the lack of systemic effects is beneficial for both female and male subjects, this is of particular importance for use in male subjects in order not to disturb the hormonal balance.

Aspect 7. The topical composition for use according to any one of the preceding aspects, wherein said composition further comprises a permeation enhancer that enables permeation through the stratum corneum.

Aspect 8. The topical composition for use according to any one of the preceding aspects, wherein said composition is for use in treating and/or preventing female and/or male hair loss, more particularly female and/or male androgenetic alopecia.

Aspect 9. The topical composition for use according to any one of the preceding aspects, wherein said composition comprises of from about 0.02% to about 1.5% (w/w) of an estetrol component, more particularly of about 0.05 to 1.2% (w/w), even more particularly of from about 0.09% to about 1.1% (w/w), or from about 0.1% to about 1% (w/w) for topical use in preventing or treating hair loss, preferably female and/or male hair loss, more preferably female and/or male androgenetic alopecia.

Aspect 10. The topical composition for use according to any one of the preceding aspects, wherein said composition comprises from about 0.05 to about 1.3% (w/w), more particularly from about 0.08 to about 1.2% (w/w), even more particularly of from about 0.09% to about 1.1% (w/w), or from about 0.1% to about 1% (w/w) of an estetrol component. Alternatively, the topical composition comprises from about 0.03% to about 0.75% (w/w) of an estetrol component, preferably of from about 0.04% to about 0.5% (w/w) of an estetrol component, more preferably of from about 0.05% to about 0.25% (w/w) of an estetrol component, most preferably about 0.06% (w/w) of an estetrol component.

Aspect 11. The topical composition for use according to any one of the preceding aspects, wherein said composition is in the form of a formulation selected from the group consisting of: solutions, emulsions, suspensions, ointments, pastes, lotions, gels (including hydrogels), foams, mousses, creams, and sprays.

Aspect 12. The topical composition for use according to any one of the preceding aspects, wherein said composition is a hydrogel that is characterised by a favourable release profile, e.g. more favourable when compared to other formulations such as but not limited to creams.

Aspect 13. The topical composition for use according to any one of the preceding aspects, wherein said composition is characterised by a mean cumulative amount of estetrol component released of at least about 2.5 μg/cm², at least about 5 μg/cm², at least about 7 μg/cm², at least about 10 μg/cm², at least about 15 μg/cm², at least about 20 μg/cm², at least about 25 μg/cm², preferably at least about 50 μg/cm², more preferably at least 100 μg/cm², more preferably at least about 150 μg/cm², more preferably at least about 200 μg/cm² within the square root of 1 hour across an isopore membrane in a receptor solution of 40:30:30 v/v/v ethanol:PEG400:water.

Aspect 14. The topical composition for use according to any one of the preceding aspects, wherein said composition is characterised by a mean cumulative amount of estetrol component released of at least about 50 μg/cm2, at least about 100 μg/cm2, at least about 150 μg/cm2, at least about 200 μg/cm2, at least about 250 μg/cm2 at least about 300 μg/cm2, at least about 350 μg/cm2, at least about 400 μg/cm2, at least about 450 μg/cm2 within 8 hours across an isopore membrane in a receptor solution of 40:30:30 v/v/v ethanol:PEG400:water. More preferably, the hydrogel described herein is characterised by a mean cumulative amount of estetrol component released of at least about 80 μg/cm2.

Aspect 15. The topical composition for use according to any one of the preceding aspects, wherein said composition is characterised by a mean % applied dose of the estetrol component into a receptor solution of 40:30:30 v/v/v ethanol:PEG400:water of at least about 15%, preferably at least about 20% after 1 hour and/or a mean % applied dose of the estetrol component into a receptor solution of 40:30:30 v/v/v ethanol:PEG400:water of at least about 40%, preferably at least about 50%, more preferably at least 80% after 8 hours and most preferably at least 90% after 8 hours.

Aspect 16. The topical composition for use according to any one of the preceding aspects, wherein said composition comprises a permeation enhancer in an amount of from about 0.5% to about 60% (w/w).

Aspect 17. The topical composition for use according to aspect 16, wherein said permeation enhancer comprises a substance or molecule that enables permeation through the stratum corneum (i.e. a permeation enhancing molecule), and a solvent.

Aspect 18. The topical composition for use according to aspect 16 or 17, wherein said permeation enhancer is selected from the group consisting of: ethanol, ether such as diethylene glycol monoethyl ether (Transcutol®), benzyl alcohol, fatty acids and esters thereof, or any combination thereof.

Aspect 19. The topical composition for use according to any one of aspects 16 to 18, wherein said permeation enhancer comprises a solvent comprising one or more polyethylene glycols (PEG), propylene glycol (PG), or combinations thereof.

Aspect 20. The topical composition for use according to aspect 19, wherein said PEG is a PEG having a molecular weight of between about 200 g/mol and about 600 g/mol such as a PEG selected from the group consisting of: PEG200, PEG300, PEG400, PEG500, PEG600, or any combination thereof.

Aspect 21. The topical composition for use according to aspect 19 or 20, wherein said permeation enhancer comprises a solvent comprising a PEG having a molecular weight of about 400 g/mol such as PEG400. In some aspects, the permeation enhancer comprises up to 50% PEG400 (w/w) and/or from about 15% to 45% PEG400 (w/w), and/or from about 20% to 40% PEG400 (w/w), and/or from about 30% to 35% PEG400 (w/w).

Aspect 22. The topical composition for use according to any one of the preceding aspects, wherein said composition comprises benzyl alcohol, preferably in an amount of from about 1% to about 3%.

Aspect 23. The topical composition for use according to any one of the preceding aspects, wherein said composition comprises a thickener.

Aspect 24. The topical composition for use according to aspect 23, wherein said composition comprises a thickener in an amount of from about 0.3% to about 20% (w/w).

Aspect 25. The topical composition for use according to aspect 23 or 24, wherein said composition comprises a thickener selected from the group consisting of a hydroxyethyl cellulose (HEC), a carboxymethyl cellulose (CMC), a high molecular weight cross-linked acrylic based polymer, a non-ionic triblock copolymer, or any combination thereof.

Aspect 26. The topical composition for use according to aspect 25, wherein the high molecular weight cross-linked acrylic based polymer is Carbopol®.

Aspect 27. The topical composition for use according to aspect 25, wherein the HEC is HEC250 HHX.

Aspect 28. The topical composition for use according to aspect 25, wherein the non-ionic triblock copolymer has an approximate molecular mass of between about 1800 and about 4000 and a polyoxyethylene content of from about 70 to about 80%.

Aspect 29. The topical composition for use according to aspect 25, wherein the non-ionic triblock copolymer is selected from a poloxamer, such as poloxamer 188, poloxamer 407, or a combination thereof.

Aspect 30. The topical composition for use according to any one of the preceding aspects, wherein said composition comprises a preservative.

Aspect 31. The topical composition for use according to aspect 30, wherein said composition may comprise a preservative in an amount of from about 1% to about 10% (w/w), preferably of from about 1% to about 3% (w/w).

Aspect 32. The topical composition for use according to aspect 30 or 31, wherein the preservative is selected from the group consisting of: lysozyme, nisin, quaternary ammonium preservatives, parabens, phenoxyethanol, benzyl alcohol, chlorobutanol, phenol, sorbic acid, thimerosal, natural preservatives, and any combination thereof.

Aspect 33. The topical composition for use according to any one of the preceding aspects, wherein said composition comprises an emollient in an amount of from about 2.5% to about 30% (w/w), preferably of from about 8% to about 12% (w/w), most preferably in an amount of about 10% (w/w).

Aspect 34. The topical composition for use according to aspect 33, wherein said composition comprises the emollient is selected from the group consisting of: glycerol, acetyl alcohol, stearyl alcohol, stearic acid, isopropyl palmitate, squalene, lanolin, glycerin, petrolatum, petroleum, and any combination thereof.

Aspect 35. The topical composition for use according to any one of the preceding aspects, wherein said composition comprises in addition to the estetrol component a permeation enhancer, a thickener, and as optional ingredient a preservative and/or an emollient, which are each preferably selected from the groups described herein.

Aspect 36. The topical composition for use according to any one of the preceding aspects, wherein said composition is complemented to 100% (w/w) by means of an aqueous solution such as water.

Aspect 37. The topical composition for use according to any one of the preceding aspects, wherein said composition comprises in addition to the estetrol component (w/w):
from about 0.1% to about 60% (w/w) of a permeation enhancer, preferably wherein said permeation enhancer comprises a permeation enhancer molecule and a solvent or solvent system;
from about 0.3% to about 20% (w/w) of a thickener;
optionally a preservative and/or an emollient;
water up to 100% (w/w).

Aspect 38. The topical composition for use according to aspect 37, wherein said composition comprises in addition to the estetrol component (w/w):
from about 16% to about 20% (w/w) PEG400;
from about 18% to about 22% (w/w) PG;
from about 8% to about 12% (w/w) glycerol;
from about 1% to about 2% (w/w) HEC; and
from about 1.5% to about 2.5% (w/w) benzyl alcohol.

Aspect 39. The topical composition for use according to aspect 38, wherein said composition comprises in addition to the estetrol component:
from about 18% to about 22% (w/w) PEG400;
from about 0.1% to about 1% (w/w) Carbopol®; and
from about 4% to about 6% (w/w) Transcutol®.

Aspect 40. The topical composition for use according to any one of the preceding aspects, wherein said prevention or treatment comprises or results in improvement in hair texture, quality and appearance, preferably wherein said prevention or treatment comprises or results in an improvement in target area hair count (TAHC) and/or target area hair width (TAHW).

Aspect 41. The topical composition for use according to any one of the preceding aspects, wherein the hair loss is caused by a hair disorder in an adult female or male subject.

Aspect 42. The topical composition for use according to any one of the preceding aspects, wherein said hair loss is female or male pattern hair loss (respectively abbreviated as FPHL and MPHL, the latter interchangeably referred to in the art as male pattern balding) or female or male androgenetic alopecia (respectively abbreviated as FAGA and MAGA).

Aspect 43. The topical composition for use according to any one of the preceding aspects, wherein the male pattern hair loss is classified as, or considered to be, male pattern baldness selected from stages 1 to 7 on the Hamilton-Norwood scale, preferably selected from stages 3 to 7 on the Hamilton-Norwood scale, more preferably stage 5, 6, or 7 on the Hamilton-Norwood scale.

Aspect 44. The topical composition for use according to any one of the preceding aspects, wherein said preventing or treating includes preventing hair loss, reversing hair loss, slowing down hair loss, reducing hair loss, increasing hair growth, or leads to an improvement in hair texture, quality and/or appearance.

Aspect 45. The topical composition for use according to any one of the preceding aspects, wherein said preventing or treating increases keratinocyte function and/or growth, and connected thereto increases function and/or growth, preferably of epidermal keratinocytes (KCs) such as those residing in the bulge, the outer root sheet (ORS), or the hair bulb.

Aspect 46. The topical composition for use according to any one of the preceding aspects, wherein said preventing or treating increases hair follicle mesenchymal fibroblast function and/or growth, and connected thereto increases the function and/or growth of dermal papilla (DP) cells (i.e. a differentiated hair inducing specialised form of fibroblasts).

Aspect 47. The topical composition for use according to any one of the preceding aspects, wherein said preventing or treating implies delaying the onset of catagen or preventing catagen.

Aspect 48. The topical composition for use according to any one of the preceding aspects, wherein said preventing or treating implies promoting and/or prolonging anagen.

Aspect 49. The topical composition for use according to any one of the preceding aspects, wherein said preventing or treating implies restoring and/or promoting the epithelial-mesenchymal interaction between the hair follicle cells.

Aspect 50. The topical composition for use according to any one of the preceding aspects, wherein said preventing or treating is characterised reducing inflammation and/or oxidative stress in hair follicles.

Aspect 51. The topical composition for use according to any one of the preceding aspects, wherein said estetrol component is estetrol or an ester thereof, preferably estetrol, either in anhydrous form or as a (mono) hydrate.

Aspect 52. The topical composition for use according to any one of the preceding aspects, wherein said estetrol component is estetrol monohydrate.

Aspect 53. The topical composition for use according to any one of the preceding aspects, wherein a further active ingredient suitable for preventing or treating hair loss is present in the topical composition, or a further active ingredient suitable for preventing or treating hair loss is co-administered or administered before or after treatment with the topical composition.

Aspect 54. A packaging unit comprising the topical composition described in any one of the preceding aspects, the packaging unit contains preferably one or more dosage units of the topical composition described herein. Suitable packaging units include any container that is capable of enclosing and preserving liquids. Optionally, the packaging unit is a box, a display unit, an ampoule, a bottle, a vial, a tube, a syringe, a cartridge, a bag, a sachet, a pouch, a film, a laminate, a foil, a can, a cylinder, or a pressurized container.

The above and further aspects and embodiments of the invention are described in the following sections and in the appended claims. The subject matter of the appended claims is hereby specifically incorporated in this specification.

### BRIEF DESCRIPTION OF THE FIGURES

FIG. 1. Mean cumulative amount of estetrol monohydrate released per unit area (μg/cm2) between the 1 and 8 h experimental period (presented as a square root of time) from 10 formulations across an isopore membrane in receptor solution 40:30:30 v/v/v ethanol:PEG400:water. Error bars represent the standard deviation of the mean (n=6).

FIG. 2. Mean cumulative amount of estetrol monohydrate released per unit area (μg/cm2) between the 1 and 8 h experimental period (presented as a square root of time) from 7 aqueous gel formulations across an isopore membrane in receptor solution 40:30:30 v/v/v ethanol:PEG400:water. Error bars represent the standard deviation of the mean (n=6).

FIG. 3. Mean cumulative amount of estetrol monohydrate released per unit area (μg/cm2) between the 1 and 8 h experimental period (presented as a square root of time) from 3 cream formulations across an isopore membrane in receptor solution 40:30:30 v/v/v ethanol:PEG400:water. Error bars represent the standard deviation of the mean (n=6).

FIG. 4. Mean percentage (%) applied dose of estetrol monohydrate released in to the receptor solution between the 1 and 8 h experimental period (presented as a square root of time) from 4 aqueous gel formulations across an isopore membrane in receptor solution 40:30:30 v/v/v ethanol:PEG400:water. Error bars represent the standard deviation of the mean (n=5-6).

FIG. 5. Mean cumulative amount of estetrol monohydrate released per unit area (μg/cm2) between the 1 and 8 h experimental period (presented as a square root of time) from 4 aqueous gel formulations across an isopore membrane in receptor solution 40:30:30 v/v/v ethanol:PEG400:water. Error bars represent the standard deviation of the mean (n=5-6).

FIG. 6. Effect of topical placebo (PBO), EstroGel® (EG), AG24, AG25 and AG26 application on uterine weight and morphology. Eight-weeks-old female mice were injected subcutaneously with LPS 24 h and 2 h prior to wounding (6 mice per group). EstroGel®, AG24, AG25, AG26 or placebo were applied in a thin layer over the top of the wound one day before, at the time of wounding and at 1, 2, 3 and 4 days after the wounding. Uterine morphological changes at 5 days were assessed by measuring uterine weight (A) and taking uterine photos (B). Scale bar=10 mm. Results are presented as mean±s.e.m. Differences versus placebo were determined using paired t-test; * P-values≤0.05, and *** P-values≤0.001.

FIG. 7. Effect of topical placebo (PBO), EstroGel® (EG), AG26 and AG28 application on uterine weight and morphology. Eight-weeks-old female mice were injected subcutaneously with LPS 24 h and 2 h prior to wounding (6 mice per group). On day 0 only (single administration), or on days −1, 0, 1 and 2 (repeated administration), EstroGel®, AG26, AG28 or their placebos were applied in a thin layer over the top of the wound. Uterine morphological changes at 3 days were assessed by measuring uterine weight. Results are presented as mean±s.e.m. Differences versus placebo were determined using paired t-test; * P-values≤0.05, and *** P-values≤0.001.

FIG. 8. Graphical representation of the antioxidant activity of E4 measured with Skin-Biosense® sensor. Antioxidant activity is evaluated by mixing a H₂O₂ 5 mM solution with E4 solutions at different concentrations and measuring the residual peak area. EUK-134 5 mM solution is used as a reference compound.

FIG. 9. Experimental design of Example 7. Microdissected human anagen VI scalp HFs were cultured at 37° C. with 5 CO₂ in a minimal media of William's E media (Gibco, Life Technologies) supplemented with 10 ng/ml hydrocortisone (Sigma Aldrich), 10 μg/ml insulin (Sigma Aldrich) and 1% penicillin/streptomycin mix (Gibco) to make Williams Complete Media (WCM) as previously described (Edelkamp et al., Molecular Dermatology, 2020 and Langan et al., Exp Dermatol, 2015).

FIG. 10. Hair cycle stages. Top two images: anagen stage. Middle two images: early catagen stages. DP: dermal papilla; DPst: dermal papilla stalk; CTS: connective tissue sheath; HM: hair matrix. Bottom image: representative image of a dystrophic hair follicle (Keyence VHX900).

FIG. 11. Representative image for hair matrix keratinocytes proliferation and apoptosis quantification. Quantitative evaluation of germinative hair matrix keratinocytes is calculated as the percentage of Ki-67 positive nuclei below the Auber's line. Intra-follicular apoptosis is analysed using TUNEL (TdT-mediated dUTP-biotin nick-end labelling) in the germinative and precortical hair matrix cells. Ki-67 staining is labelled in red, TUNEL in green, and nuclei (DAPI) in blue. gHM: germinative hair matrix; pcHM: precortical hair matrix.

FIG. 12. Estetrol does not induce LDH release into the medium. Medium pooled from n=7-8 HFs/group cultured until day 6 from Donor 2 (Day 1, Day 3, Day 5 and 6); Mean, GraphPad Prism 9, D'Agostino & Pearson omnibus normality test. A non-parametric analysis was applied, Kruskal Wallis test, Dunn's multiple comparison test—Vehicle fixed, not significant.

FIG. 13. Estetrol does not impact hair shaft production. Pool data of n=4 female donors, Mean±SEM, GraphPad Prism 9. Kruskal-Wallis test p=0.4856; Dunn's multiple comparison test (vehicle fixed) ns; Mann-Whitney vs vehicle *p<0.05. Different grey intensities of the samples are indicative for different donors.

FIG. 14. Microscopically, all estetrol concentrations (3 μM in particular) maintain longer HFs in anagen. (A) Microscopic hair cycle staging. (B) Microscopic hair cycle score. Pool data of n=4 female donors, Mean±SEM, GraphPad Prism 9. Kruskal-Wallis test *p=0.0304; Dunn's multiple comparison test (vehicle fixed) ns; Mann-Whitney vs vehicle *p<0.05. Different grey intensities of the samples are indicative for different donors. (C, D) Microscopic images: DP: dermal papilla; HMx: hair matrix; DC: dermal cup. displayed scale bar indicates 100 μm.

FIG. 15. Estetrol (3 μM) increases hair matrix keratinocytes proliferation, whilst apoptosis was not affected (A, B). Pool data of n=4 female donors, Mean±SEM, GraphPad Prism 9. (A): one-way ANOVA p=0.2115; Holm-Sidak's multiple comparison test vehicle fixed ns; Unpaired student's t-test vs vehicle *p<0.05. Different grey intensities of the samples are indicative for different donors. (B): Kruskal-Wallis test p=0.6460; Dunn's multiple comparison test (vehicle fixed) ns; Mann-Whitney vs vehicle; ns. (C) Microscopic images: Ki-67+ cells were counted in the demarcated areas below the Auber's line (germinative hair matrix, gHM) while TUNEL+ cells were counted in the demarcated areas below (gHM) and above (precortical hair matrix, pcHM) the Auber's line.

FIG. 16. All tested concentrations of E4 tendentially increased versican expression and alkaline phosphatase activity in dermal papilla (DP) cells. Pool data of n=4 female donors, Mean±SEM, GraphPad Prism 9 (A) versican expression in the dermal papilla. Kruskal-Wallis test p=0.3529; Dunn·s multiple comparison test (vehicle fixed) ns; Mann-Whitney vs vehicle ns. Different grey intensities of the samples are indicative for different donors. (B) Alkaline phosphatase activity in the dermal papilla. Kruskal-Wallis test p=0.5508; Dunn's multiple comparison test (vehicle fixed) ns; Mann-Whitney vs vehicle ns. (C) Microscopic images. Top: versican is labelled in green and nuclei (DAPI) in blue; below: alkaline phosphatase is labelled in blue. DP=dermal papilla.

FIG. 17. DP fibroblast emigration analysis revealed that all tested concentrations of E4 did not strikingly affect the density of cells in the DP. Pool data of n=4 female donors, Mean±SEM, GraphPad Prism 9. Kruskal-Wallis test p=0.2081, Dunn's multiple comparison test (vehicle fixed) ns; Mann-Whitney vs vehicle ns. Different grey intensities of the samples are indicative for different donors.

FIG. 18. All Estetrol concentrations tendentially decreased the density of cells and 300 nM and 3 μM E4 significantly reduced the total number of cells in the DP stalk. Pool data of n=4 female donors Mean±SEM, GraphPad Prism 9. (A) Density of dermal papilla stalk cells. one-way ANOVA p=0.5092; Holm-Sidak's multiple comparison test vehicle fixed ns; Unpaired student's t-test vs vehicle ns. Different grey intensities of the samples are indicative for different donors. (B) Total number of dermal papilla stalk cells. one-way ANOVA *p=0.0296; Holm-Sidak's multiple comparison test vehicle fixed *p=0.0177; Unpaired student's t-test vs vehicle, *p<0.05.

FIG. 19. Estetrol had no significant impact on the density of cells in the inductive dermal cup. Pool data of n=4 female donors, Mean±SEM, GraphPad Prism 9. One-way ANOVA p=0.7616; Holm-Sida k's multiple comparison test vehicle fixed ns; Unpaired student's t-test vs vehicle ns. Different grey intensities of the samples are indicative for different donors.

FIG. 20. In the bulge basal layer, E4 3 μM significantly decreased the percentage of K15 positive cells, whilst E4 30 μM (significantly) and 3 μM and 300 nM (tendentially) increased their proliferation. Pool data of n=4 female donors, Mean±SEM, GraphPad Prism 9. (A) Percentage of K15+ cells in the bulge basal layer. One-way ANOVA p=0.0641; Holm-Sidak's multiple comparison test vehicle fixed #p=0288; Unpaired student's t-test vs vehicle *p<0.05 Different grey intensities of the samples are indicative for different donors. (B) Percentage of Ki-67+ cells in the bulge basal layer. Kruskal-Wallis test p=0.1302; Dunn's multiple comparison test (vehicle fixed) ns; Mann-Whitney vs vehicle ** p<0.01.

FIG. 21. In the suprabulbar ORS, estetrol did not have any effect on the percentage of K15 positive cells, but E4 300 nM (significantly) and 3 μM (tendentially) decreased their proliferation. Pool data of n=4 female donors, Mean±SEM, GraphPad Prism 9. (A) percentage of K15+ cells in the suprabulbar basal layer. Kruskal-Wallis test p=0.9375; Dunn's multiple comparison test (vehicle fixed) ns; Mann-Whitney vs vehicle ns. Different grey intensities of the samples are indicative for different donors. (B) Percentage of Ki-67+ cells in the suprabulbar basal layer. Kruskal-Wallis test p=0.7462; Dunn's multiple comparison test (vehicle fixed) ns; Mann-Whitney vs vehicle *p<0.05. (C) Microscopic images. Suprabulbar ORS is delimited with dotted line. Scale bar=100 μm.

FIG. 22. Estetrol 3 μM (significantly) and 300 nM and 30 μM (tendentially) increased the percentage of CD34 positive cells in the suprabulbar ORS. (A) percentage of CD34+ cells in the bulge basal layer. Pool data of n=4 female donors, Mean±SEM, GraphPad Prism 9 One-way A NOVA p=0.1011; Holm-Sida k's multiple comparison test vehicle fixed *p=0.0392; Unpaired student's t-test vs vehicle *p<0.05. Different grey intensities of the samples are indicative for different donors. (B) Microscopic images. Suprabulbar ORS is delimited with dotted line. Scale bar=100 μm (ORS: outer root sheath).

FIG. 23. Experimental design of Example 8. Microdissected human anagen VI scalp HFs were cultured at 37° C. with 5% CO₂ in a minimal media of William's E media (Gibco, Life Technologies) supplemented with 10 ng/ml hydrocortisone (Sigma Aldrich). 10 μg/ml insulin (Sigma Aldrich) and 1% penicillin/streptomycin mix (Gibco) to make Williams Complete Media (WCM) modified as previously described (Edelkamp et al., Molecular Dermatology 2020; Langan et al., Exp Dermatol 2015).

FIG. 24. —Reduction of hair shaft production by estradiol and tendentially by estetrol 300 nM and 3 μM. Top line: vehicle; two middle lines: 300 nM estradiol and 300 nM estetrol; bottom line: 3 μM estetrol. n=5-6 HFs/group (all HFs) from one healthy male donor; mean±SEM GraphPad Prism 9; Mann-Whitney test, **p<0.01. Dunn·s multiple comparison test—vehicle fixed, ns.

FIG. 25. Tested concentrations of estetrol maintain hair follicles in anagen even more prominently with 3 μM estetrol whilst estradiol does not affect hair cycle. n=5-6 HFs/group from one healthy male donor, Mean±SEM, GraphPad Prism 9. Kruskal-Wallis test p=0.1723; Dunn's multiple comparison test (vehicle fixed); ns Mann-Whitney vs vehicle, ns.

FIG. 26. Hair matrix keratinocyte proliferation is increased by 300 nM estradiol and 3 μM estetrol. 4-6 HFs/group (all HFs) from one healthy male donor, Mean±SEM, GraphPad Prism 9. Ki-67+ cells were counted in the demarcated areas below the Auber's line (germinative hair matrix) while TUNEL+ cells were counted in the demarcated areas below and above (precortical hair matrix) the Auber's line. Kruskal-Wallis test p=0.2571; Dunn's multiple comparison test (vehicle fixed), ns; Mann-Whitney vs vehicle, ns.

FIG. 27. Estrogens decrease versican expression in the dermal papilla. 5-6 HFs/group (all HFs) from one healthy male donor, Mean±SEM, Graph Pad Prism 9. Kruskal-Wallis test p=0.3137; Dunn's multiple comparison test vehicle fixed, ns; Mann-Whitney vs vehicle, ns.

FIG. 28. Estrogens decrease alkaline phosphatase activity in the dermal papilla. 5-6 HFs/group (all HFs) from one healthy male donor, Mean±SEM, Graph Pad Prism 9. Kruskal-Wallis test p=0.1395; Dunn's multiple comparison test vehicle fixed, ns; Mann-Whitney vs vehicle, ns.

FIG. 29. 3 μM estetrol significantly decreases the density (top panel) and the total number of cells (bottom panel) in the inductive dermal cup in anagen hair follicles. Estradiol does not display a significant effect. n=2-6 HFs/group (only anagen) from one healthy male donor, Mean±SEM, GraphPad Prism 9. Top graph: Kruskal-Wallis test *p=0.0335; Dunn's multiple comparison test vehicle fixed, ns; Mann-Whitney vs vehicle, *p<0.05. Bottom graph: Kruskal-Wallis test p=0.0826; Dunn's multiple comparison test vehicle fixed, #p<0.05; Mann-Whitney vs vehicle, *p<0.05.

FIG. 30. Estetrol tendentially decreases the density (top panel) and the total number of cells (middle panel) in the dermal papilla (3 μM) while it increases the area (300 nM and 3 μM) (bottom panel). Estradiol (E2) does not display a significant effect. 5-6 HFs/group (all hair follicles) from one healthy male donor, Mean±SEM, GraphPad Prism 9. Top graph: Kruskal-Wallis test p=0.0514; Dunn's multiple comparison test vehicle fixed, ns; Mann-Whitney vs vehicle, ns. Middle graph: Kruskal-Wallis test p=0.1671; Dunn's multiple comparison test vehicle fixed, ns; Mann-Whitney vs vehicle, ns. Bottom graph: Kruskal-Wallis test p=0.2226; Dunn's multiple comparison test vehicle fixed, ns; Mann-Whitney vs vehicle, ns.

FIG. 31. 3 μM Estetrol decreases the density of cells in the dermal papilla stalk (top panel) as well as tendentially the total number of dermal papilla stalk cells (bottom panel). Estradiol does not display a significant effect. 5-6 HFs/group (all hair follicles) from one healthy male donor, Mean±SEM, GraphPad Prism 9. Top graph: Kruskal-Wallis test *p=0.0190; Dunn's multiple comparison test vehicle fixed, #p<0.05; Mann-Whitney vs vehicle, *p<0.05. Bottom graph: Kruskal-Wallis test p=0.3056; Dunn's multiple comparison test vehicle fixed, ns; Mann-Whitney vs vehicle, ns.

FIG. 32. Estetrol decreases the number of CD34+ cells in the suprabulbar outer root sheath whilst 300 nM estradiol does not display any effect. 5-6 HFs/group (all HFs) from one healthy male donor, Mean±SEM, GraphPad Prism 9. Kruskal-Wallis test *p=0.0401; Dunn's multiple comparison test vehicle fixed, #p<0.05; Mann-Whitney vs vehicle, *p<0.05.

FIG. 33. Estradiol and estetrol at 300 nM tendentially increase K15 expression (top panel) as well as the number of cells in the suprabulbar outer root sheath (bottom panel). n=S-6 HFs/group from one healthy male donor, Mean±SEM, GraphPad Prism 9. ORS: outer root sheath. Top graph: Kruskal-Wallis test p=0.1435; Dunn's multiple comparison test vehicle fixed, ns; Mann-Whitney vs vehicle, ns. Bottom graph: Kruskal-Wallis test p=0.5110; Dunn's multiple comparison test vehicle fixed, ns; Mann-Whitney vs vehicle, ns.

FIG. 34. Representative brightfield images of terminal and intermediate hair follicles from affected and non-affected areas. DP=dermal papilla, FUE=follicle unit extraction.

FIG. 35. Experimental design of Example 9. Microdissected human anagen VI hair follicles from FUEs were cultured at 37° C. with 5% CO₂ in a minimal media of William's E media (Gibco, Life Technologies) supplemented with 2 mM of L-glutamine (Gibco). 10 ng/ml hydrocortisone (Sigma Aldrich). 10 μg/ml insulin (Sigma Aldrich) and 1% penicillin/streptomycin mix (Gibco) to make Williams Complete Media (WCM) as previously described (Edelkamp et al., Molecular Dermatology 2020; Langan et al., Exp Dermatol 2015).

FIG. 36. 3 μM estetrol prolongs anagen in hair follicles from nonaffected (terminal) and affected (terminal and intermediate) scalp areas of FPHL patients. Pool data of n=2 FPHL donors, n=2-6 HFs/group, Mean±SEM, GraphPad Prism 9. Microscopic hair cycle staging was performed according to the criteria published in Langan et al., Exp Dermatol 2015. Bottom graph: Kruskal-Wallis test multiple comparison, p=0.5532; Dunn's multiple comparison test—vehicle fixed, ns; Mann-Whitney test—vehicle fixed, ns.

FIG. 37. 3 μM estetrol tendentially increases hair matrix keratinocytes proliferation in terminal hair follicle from non-affected and from affected scalp of FPHL patients, without affecting hair matrix keratinocytes apoptosis. Pool data of n=2 FPHL donors, n=2-6 HFs/group, Mean±SEM, GraphPad Prism 9. Ki-67+ cells (top panel) were counted in the demarcated areas below the Auber's line (germinative hair matrix) while TUNEL+ cells (bottom panel) were counted in the demarcated areas below and above (precortical hair matrix) the Auber's line. Top graph: Kruskal-Wallis test multiple comparison, p=0.2859; Dunn's multiple comparison test—vehicle fixed, ns; Mann-Whitney test—vehicle fixed, ns. Bottom graph: Kruskal-Wallis test multiple comparison, p=0.5686; Dunn's multiple comparison test—vehicle fixed, ns; Mann-Whitney test—vehicle fixed, ns.

FIG. 38. 3 μM estetrol tendentially increases versican expression in terminal and intermediate hair follicles from non-affected scalp. Pool data of n=2 FPHL donors, n=2-6 HFs/group, Mean±SEM, GraphPad Prism 9. Kruskal-Wallis test multiple comparison, p=0.2620; Dunn's multiple comparison test—vehicle fixed, ns; Mann-Whitney test—vehicle fixed, ns.

FIG. 39. 3 μM estetrol significantly reduces (terminal) whilst increases (intermediate) the total number of cells in the dermal cup of hair follicles from non-affected area. Pool data of n=2 FPHL donors, n=2-6 HFs/group, Mean±SEM, GraphPad Prism 9. Kruskal-Wallis test multiple comparison, p=0.7056; Dunn's multiple comparison test—vehicle fixed, ns; Mann-Whitney test—vehicle fixed, *p<0.05.

FIG. 40. Treatment with 3 μM estetrol tendentially decreases the density (bottom panel) of dermal papilla (DP) cells of intermediate hair follicle from the non-affected area whilst increases the total DP cell number (top panel) and the DP area size (middle panel). The density and total number of DP cells of intermediate hair follicles from the affected area is tendentially increased by 3 μM E4. Pool data of n=2 FPHL donors, n=2-6 HFs/group, Mean±SEM, GraphPad Prism 9. Top graph: Kruskal-Wallis test multiple comparison, p=0.4256; Dunn's multiple comparison test—vehicle fixed, ns; Mann-Whitney test—vehicle fixed, ns. Middle graph: Kruskal-Wallis test multiple comparison, p=0.9683; Dunn's multiple comparison test—vehicle fixed, ns; Mann-Whitney test—vehicle fixed, ns. Bottom graph: Kruskal-Wallis test multiple comparison, p=0.2878; Dunn's multiple comparison test—vehicle fixed, ns; Mann-Whitney test—vehicle fixed, ns.

FIG. 41. 3 μM estetrol significantly decreases the total number (top panel) and area size (middle panel) of dermal papilla stalk cells in terminal hair follicles from the non-affected area, without influencing their density (bottom panel). In intermediate hair follicles from the non-affected area, the density of dermal papilla stalk cells is tendentially decreased. Pool data of n=2 FPHL donors, n=2-6 HFs/group; n=I-4 anagen HFs/group, Mean±SEM, Graph Pad Prism 9. Top graph: Kruskal-Wallis test multiple comparison, p=0.2890; Dunn's multiple comparison test—vehicle fixed, ns; Mann-Whitney test—vehicle fixed, *p<0.05. Middle graph: Kruskal-Wallis test multiple comparison, p=0.4812; Dunn's multiple comparison test—vehicle fixed, ns; Mann-Whitney test—vehicle fixed, **p<0.01. Bottom graph: Kruskal-Wallis test multiple comparison, p=0.6340; Dunn's multiple comparison test—vehicle fixed, ns; Mann-Whitney test—vehicle fixed, ns.

FIG. 42. Estetrol tendentially increased K15 expression and number of positive cells in terminal HFs from the non-affected scalp in the suprabulbar outer root sheath, and increased proliferation of K15+ cells in terminal HFs from non-affected and intermediate HFs from affected scalp. Pool data of n=2 FPHL donors, n=2-6 HFs/group, Mean±SEM, Graph Pad Prism 9. Top graph: Kruskal-Wallis test multiple comparison, p=0.6601; Dunn's multiple comparison test—vehicle fixed, ns; Mann-Whitney test—vehicle fixed, ns. Middle graph: Kruskal-Wallis test multiple comparison, p=0.8527; Dunn's multiple comparison test—vehicle fixed, ns; Mann-Whitney test—vehicle fixed, ns. Bottom graph: Kruskal-Wallis test multiple comparison, p=0.7225; Dunn's multiple comparison test—vehicle fixed, ns; Mann-Whitney test—vehicle fixed, ns.

FIG. 43. Estetrol tendentially increased K15 expression in intermediate HFs from the non-affected scalp, and increased proliferation of K15+ cells in terminal HFs from affected scalp. Pool data of n=2 FPHL donors, n=1-6 HFs/group, Mean±SEM, Graph Pad Prism 9. Top graph: Kruskal-Wallis test multiple comparison, p=0.6091; Dunn's multiple comparison test—vehicle fixed, ns; Mann-Whitney test—vehicle fixed, ns. Bottom graph: Kruskal-Wallis test multiple comparison, p=0.8852; Dunn's multiple comparison test—vehicle fixed, ns; Mann-Whitney test—vehicle fixed, ns.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from . . . to . . . ” or the expression “between . . . and . . . ” or another expression.

The terms “about” or “approximately” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

The term “estetrol component”, as used throughout this document, encompasses substances selected from the group consisting of estetrol, esters of estetrol, esters of estetrol wherein the hydrogen atom of at least one of the hydroxyl groups has been substituted by an acyl radical of a hydrocarbon carboxylic, sulfonic acid or sulfamic acid of 1-25 carbon atoms, anhydrous estetrol, estetrol hydrates such as estetrol monohydrate; and combinations thereof. It is understood that when estetrol is mentioned throughout any section of this specification, any estetrol-containing component (i.e. compound) and/or estetrol derivative (such as an estetrol ester or hydrate) is also envisaged. More preferably, in the context of the present disclosure, a particularly preferred estetrol component suitable for the dosage unit or the cosmetic or medical uses and methods of treatment described herein is estetrol (anhydrous or estetrol hydrates). Most preferably, said estetrol component is estetrol monohydrate.

The term “estetrol” as used herein refers to 1,3,5 (10)-estratrien-3,15alpha,16alpha,17beta-tetrol or 15alpha-hydroxyestriol as well as hydrates of estetrol, e.g. estetrol monohydrate. “Estetrol”, or short “E4” is an estrogen steroid produced by the fetal human liver (PubChem CID: 27125). Estetrol may be described as a 3-hydroxy steroid corresponding to 17beta-estradiol wherein the 15 and 16a positions are substituted for two additional hydroxy groups. It is known that estetrol is an estrogen receptor agonist (Coelingh Bennink et al., Estetrol review: profile and potential clinical applications, Climacteric, 2008). In instances wherein the estetrol component described herein indicates estetrol, said estetrol may be endogenous estetrol. Alternatively, the estetrol may be chemically synthesized, synthesized by the use of (mutant) recombinant enzymes, or synthesized by any combination thereof. Estetrol may alternatively be indicated in the art by its molecular formula: C₁₈H₂₄O₄, or by structural formula (I):

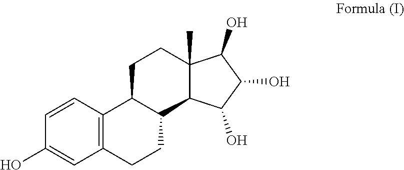 [SMILES: C[C@]12CC[C@@H]3c4ccc(O)cc4CC[C@H]3[C@@H]1[C@@H](O)[C@@H](O)[C@@H]2O]

In a preferred embodiment, the estetrol is present or used herein as a monohydrate or anhydrous estetrol, more preferably as a monohydrate estetrol. A skilled person appreciates that while estetrol monohydrate may be used as ingredient upon preparation of the topical composition described herein, in the resulting topical composition the estetrol monohydrate may be solubilized and no longer be present in a monohydrate form.

In some embodiments, the estetrol component can be the sole active ingredient or can be combined with any other cosmetic or pharmaceutically active agent for alleviating or preventing hair loss known in the art.

“(w/w)”, or alternatively indicated throughout the art by terms such as “weight by weight” or “weight for weight”, refers to the contribution of a particular molecule or substance within a composition or mixture as measured by its weight (i.e. mass).

The term “pharmaceutically active ingredient”, interchangeably used throughout the present disclosure with “pharmaceutically active agent” is to be interpreted according to the definition of the term by the World Health organisation: “a substance used in a finished pharmaceutical product (FPP), intended to display pharmacological activity or to otherwise have direct effect in the diagnosis, cure, mitigation, treatment or prevention of disease, or to have direct effect in restoring, correcting or modifying physiological functions in human beings”. The physiological effect may be achieved by one dose or by repeated doses. Unless otherwise specified, in the context of the present disclosure the term refers to the estetrol component, preferably estetrol.

As used throughout the present disclosure, the terms “therapy” or “treatment” refer to the alleviation or measurable lessening of one or more symptoms or measurable markers of a pathological condition, in the context of the present invention female or male balding (e.g. male pattern balding, female pattern hair loss, androgenetic alopecia). The hair loss may be hair loss without any further underlying medical condition, but may equally be caused by a medical condition or occur as the consequence of a medical treatment (e.g. chemotherapy). Optionally, the hair loss is, is considered to be, or is (deemed to be) caused by alopecia areata. The terms encompass both the treatment of a balding pattern that has already developed (i.e. thinning of the hair mass has already started), as well as prophylactic or preventive measures, wherein the goal of the treatment is to prevent occurrence and/or re-occurrence, development and progression of balding in a female or male subject. By means of illustration and limitation, a preventive use of the topical composition described herein may be administration to the scalp of a female or male subject that is starting a medical treatment which is known to cause hair loss. An alternative example may be application to the scalp of a subject that is expected to develop female or male pattern balding in a foreseeable future, which may be predicted by means of illustration and not limitation by the observation of female or male balding in family members, or by conducting a trichogram analysis.

Measurable lessening includes any statistically significant decline in the rate of hair thinning, hairline recession, hair loss, or any combination thereof. Statistically significant as used herein refers to p values below 0.05, which is a commonly accepted cut-off score in statistical analysis as a skilled person appreciates. More particular indications of preventing or treating hair loss are described in detail further below.

A skilled person is aware that in order to achieve an effective therapeutic treatment, a therapeutically effective dose needs to be administered to said subject. Therefore, in the context of the present disclosure “an effective amount” refers to an amount necessary to obtain a physiological effect. The physiological effect may be achieved by a single dose or by multiple doses. A “therapeutically effective amount” or “therapeutically effective dose” indicates an amount of estetrol component that when administered brings about a clinical positive response with respect to treatment of a subject. Similarly, a “prophylactically effective amount” or “prophylactically effective dose” refers to an amount of estetrol component that inhibits or delays female or male (pattern) hair loss or balding. A skilled person is aware that terms such as “quantity”, “amount” and “level” are synonyms and have a well-defined meaning in the art and appreciates that these in the context of the present application refer to a relative quantification of an estetrol component part of a topical composition such as but not limited to a hydrogel, or when indicated refer to an absolute quantification of an estetrol component which is considered an effective amount for the applications described herein upon application to the skin of a subject.

### Topical Compositions

As described in the summary of the present disclosure, topical compositions comprising an estetrol component are provided that have at least similar efficacy in preventing and treating hair loss in female and/or male subjects as topical compositions that have been described in the art. However, the topical compositions subject of the present invention display markedly diminished systemic effects, regardless of the exact formulation that is used. The diminished, and arguably even lack of systemic effects can therefore be observed for solutions, emulsions, suspensions, ointments, pastes, lotions, gels (including hydrogels), foams, mousses, creams, and sprays. This observation even remains valid upon inclusion of a permeation enhancer in the composition. This allows for the formulation of a topical composition comprising a relatively low estrogen dosage. The anti-hair loss properties of the topical composition at such low doses of an estetrol component is additionally remarkable given that estetrol has been historically considered a weak estrogen when compared to other estrogens such as estradiol (Gérard et al., J Endocrinol, 2015). Hence, the topical compositions disclosed in the present application for use in preventing and/or treating hair loss were not envisaged.

It is understood that any reference to “topical composition” encompasses any solutions, emulsions, suspensions, ointments, pastes, lotions, gels (including hydrogels), foams, mousses, creams, and sprays described herein, and vice versa.

Accordingly, a first aspect of the invention is directed to a composition comprising an effective amount of an estetrol component for topical use in preventing or treating hair loss in a subject, or for topical application, such as for topical application to the scalp. Thus, in such an aspect the invention is directed to a topical composition for use in preventing or treating hair loss in a subject. Alternatively worded, the present invention envisages the use of a topical composition comprising an effective amount of an estetrol component for preventing or treating hair loss in a subject. Yet alternatively worded, the present invention envisages the use of a composition comprising an effective amount of an estetrol component for use in the manufacture of a topical medicament for preventing or treating hair loss. A skilled person readily appreciates that in absence of any indication to the contrary, all embodiments described herein are envisaged in the context of use for preventing and treating hair loss. Combination of any embodiment with the indication of the use of preventing and/or treating hair loss should therefore not be considered to constitute a combination of separate embodiments of the description.

In particular embodiments, the topical composition comprises of from about 0.01% to about 0.18% (w/w) of an estetrol component such as for example estetrol. In preferred embodiments, the topical composition comprises of from about 0.02% to about 0.16% (w/w) of an estetrol component such as for example estetrol, preferably of from about 0.03% to about 0.14% (w/w) of an estetrol component such as for example estetrol, more preferably of from about 0.04% to about 0.12% (w/w) of an estetrol component such as for example estetrol, yet more preferably of from about 0.05% to about 0.10% (w/w) of an estetrol component such as for example estetrol, even more preferably of from about 0.05% to about 0.08% (w/w) of an estetrol component such as for example estetrol.

In alternative particular embodiments, the topical composition comprises of about 0.18% (w/w) or less of an estetrol component such as for example estetrol. In preferred embodiments, the topical composition comprises of about 0.16% (w/w) or less of an estetrol component such as for example estetrol, preferably of about 0.14% (w/w) or less of an estetrol component such as for example estetrol, more preferably of about 0.12% (w/w) or less of an estetrol component such as for example estetrol, yet more preferably of about 0.10% (w/w) or less of an estetrol component such as for example estetrol, even more preferably of about 0.08% (w/w) or less of an estetrol component such as for example estetrol.

In preferred embodiments, the estetrol is present or used herein as a monohydrate. Hence, in particular embodiments the topical composition comprises of from about 0.01% to about 0.18% (w/w) of estetrol monohydrate. In preferred embodiments, the topical composition comprises of from about 0.02% to about 0.16% (w/w) of estetrol monohydrate, preferably of from about 0.03% to about 0.14% (w/w) of estetrol monohydrate, more preferably of from about 0.04% to about 0.12% (w/w) of estetrol monohydrate, yet more preferably of from about 0.05% to about 0.10% (w/w) of estetrol monohydrate, even more preferably of from about 0.05% to about 0.08% (w/w) of estetrol monohydrate.

In alternative particular embodiments, the topical composition comprises of about 0.18% (w/w) or less of estetrol monohydrate. In preferred embodiments, the topical composition comprises of about 0.16% (w/w) or less of estetrol monohydrate, preferably of about 0.14% (w/w) or less of estetrol monohydrate, more preferably of about 0.12% (w/w) or less of estetrol monohydrate, yet more preferably of about 0.10% (w/w) or less of estetrol monohydrate, even more preferably of about 0.08% (w/w) or less of estetrol monohydrate.

In yet alternative particular embodiments the topical composition comprises of from 0.02% to 1.5% (w/w) of an estetrol component, preferably comprising of from 0.05% to 1.2% (w/w) of an estetrol component, of from about 0.02% to about 1% (w/w) of an estetrol component of from about 0.03% to about 1% (w/w) of an estetrol component, preferably of from about 0.04% to about 1% (w/w) of an estetrol component, more preferably of from about 0.05% to about 1% (w/w) of an estetrol component, most preferably from about 0.06% to about 0.5% (w/w) of an estetrol component, more preferably of from about 0.09% to about 1.1% (w/w), even more from 0.1% to 1% (w/w) of an estetrol component, most preferably of from 0.3% to 0.7% (w/w) of an estetrol component.

In yet alternative embodiments, the topical composition comprises of from about 0.01% to about 5% (w/w) of an estetrol component, preferably of from about 0.02% to about 1% (w/w) of an estetrol component, more preferably of from about 0.03% to about 0.75% (w/w) of an estetrol component, yet more preferably of from about 0.04% to about 0.5% (w/w) of an estetrol component, most preferably about 0.06% (w/w) of an estetrol component.

The above embodiments do not exclude the presence of non-estetrol estrogenic components in the topical composition. Also envisaged are different estetrol components within a single composition. In such embodiments, the topical composition may by means of illustration and not limitation comprise both estetrol or, more particularly, estetrol monohydrate and an ester of estetrol.

With “topical application” application to a particular place on the body is meant. In particular, application to body surfaces such as the skin is envisaged. When used topically, local effects are usually sought, particularly local effects on the skin. Topical application is preferably understood as application to the skin of the scalp within the present context and may include occipital and/or frontotemporal application.

A skilled person appreciates that “stratum corneum” to which reference is made herein refers to the outer layer of the epidermis that is composed of multiple layers of terminally differentiated keratinocytes made primarily of the proteinaceous material keratin. The composition, function, and particulars of the stratum corneum have been described in great detail in the art (e.g. in Matsui and Amagai, Int Immunol, 2015). It is generally considered that permeation of pharmaceutically active agents throughout the skin is rate limited by the stratum corneum. Thus, the permeation enhancers described herein aid in permeation of at least the estetrol component throughout the stratum corneum. In particular embodiments, the topical composition comprises a permeation enhancer that enables permeation through the stratum corneum. In the context of the present invention, the term “permeation enhancer”, interchangeably used with terms including but not limited to “permeability enhancer”, “permeability increaser”, “permeability inducer”, and even “skin enhancer”. It is appreciated by a skilled person that the term “permeation enhancer” upon use throughout the present disclosure indicates a permeation enhancer molecule as part of a solvent or solvent system. Different permeation enhancers have been described in detail in the art and include without limitation those listed in the CPE database (Vasyuchenko et al., Pharmaceutics, 2021). Exemplary and non-limiting permeation enhancers are described further herein.

In preferred embodiments, the topical composition comprises of from about 0.01% to about 5% (w/w), such as from about 0.08% to about 1.2% (w/w), from about 0.09% to about 1.1% (w/w), or from about 0.1% to about 1% (w/w) of an estetrol component (such as estetrol, preferably estetrol monohydrate) and a permeation enhancer, preferably of from about 0.02% to about 2.5% (w/w) of an estetrol component and a permeation enhancer, more preferably of from about 0.02% to about 2% (w/w) of an estetrol component and a permeation enhancer, more preferably of from about 0.03% to about 1.5% (w/w) of an estetrol component and a permeation enhancer, yet more preferably of from about 0.03% to about 1% (w/w) of an estetrol component and a permeation enhancer, yet more preferably of from about 0.03% to about 0.75% (w/w) of an estetrol component and a permeation enhancer, yet even more preferably of from about 0.04 to about 0.5% (w/w) of an estetrol component and a permeation enhancer, most preferably about 0.06% (w/w) of an estetrol component and a permeation enhancer.

In alternative embodiments, the topical composition comprises of from about 0.01% to about 5% or less (w/w), such as from about 0.08 to about 1.2% (w/w), from about 0.09% to about 1.1% (w/W), or from about 0.1% to about 1% (w/w) of an estetrol component (such as estetrol, preferably estetrol monohydrate) and a permeation enhancer, preferably about 2.5% (w/w) or less of an estetrol component and a permeation enhancer, more preferably about 2% (w/w) or less of an estetrol component and a permeation enhancer, more preferably about 1.5% (w/w) or less of an estetrol component and a permeation enhancer, yet more preferably about 1% (w/w) or less of an estetrol component and a permeation enhancer, yet more preferably 0.75% (w/w) or less of an estetrol component and a permeation enhancer, yet even more preferably about 0.5% (w/w) or less of an estetrol component and a permeation enhancer, most preferably about 0.1% (w/w) or less of an estetrol component and a permeation enhancer.

As is evident for a skilled person, the present disclosure encompasses the use of each of the particular forms of the topical composition disclosed herein for use in preventing or treating hair loss. Suitable topical compositions and formulations therefore include, but are not limited to solutions, emulsions, suspensions, ointments, pastes, lotions, gels (including hydrogels), foams, mousses, creams, and sprays. Each of these terms are intended to correspond to their generally accepted meaning. Similarly, these topical compositions can either be applied directly to the skin of the scalp as such, or in combination with a dressing, patch, bandage, band aid, tampon, the inside of a plaster, or the like to prevent the topical composition from being removed off the skin and in some embodiments to shield the treated area from external influences such as dirt, microorganisms, and direct sunlight. Furthermore, it is to be appreciated that due to solubilization, estetrol may not be present in the topical composition such as a solution or a foam in a monohydrate form, or only partially present in the composition as a monohydrate.

“Solution” refers to a liquid homogeneous distribution of two or more different substances in one another. A distinction is made between real and colloidal solutions. In real solutions, the dissolved substance is molecularly dispersed in the solvent, i.e. it is present in discrete single molecules. In the case of colloidal solutions, the divided particles are of colloidal size. In a solution according to the invention, one or more active pharmaceutical ingredients are dissolved in a suitable solvent or mixture of mutually miscible solvents (aqueous or non-aqueous).

“Emulsion” broadly refers to any mixture of at least two liquids that are unmixable (i.e., immiscible, unblendable) and thus wherein a first liquid is distributed in small droplets (dispersed phase) throughout a second liquid (dispersion medium). Therefore, in certain embodiments the pharmaceutical composition described herein is an oil-in-water or water-in-oil emulsion. Emulsions are widely used in skin care formulations and can be classified as creams and lotions. Related hereto, “suspension” broadly refers to a heterogeneous mixture containing solids dispersed in a liquid phase that are not dissolved and have a size which is sufficiently large to allow for sedimentation.

“Cream” generally refers to a water-in-oil emulsion wherein an aqueous phase is dispersed in an oil phase, but may equally be an oil-in-water emulsion in which an oil is dispersed within an aqueous base. It is generally accepted that creams differ from emulsions in that emulsions are stable suspensions of small immiscible droplets of fluid immiscible with another fluid part of the emulsion, while a cream instead indicates a particular subset of emulsions that are more viscous and usually include more lipophilic and/or surfactant components.

A “lotion” is a low- to medium-viscosity liquid composition. Generally, lotions are less viscous than creams, but in some cases the viscosity of both can be similar. A lotion can contain finely powdered substances that are insoluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

An “ointment” broadly generally refers to a more viscous oil-in-water cream, i.e., to a semi-solid substance containing an ointment base and optionally one or more pharmaceutically active ingredients (in the context of the present invention an estetrol component). Examples of suitable ointment bases include hydrocarbon bases, absorption bases, water-removable bases, and water-soluble bases. “Pastes” generally differ from ointments in that they contain a larger percentage of solids. Overall, pastes are more absorptive and less greasy when compared to ointments based on an identical set of ingredients/excipients.

“Foam” as used herein refers to a formation consisting of a large volume of gas dispersed in a liquid containing one or more active substances, a surfactant that ensures its formation and other ingredients. In general, a foam is a dispersion of gas in a liquid-liquid foam, or in a solid state-solid foam. Due to the size of the gas bubbles, foams are divided into the following structures: microporous (pore diameter below 2 nm), mesoporous (pore diameter 2-50 nm) and macroporous (pore diameter above 50 nm). Moreover, due to the pore size distribution, they can be divided into monodisperse foams or polydisperse foams. The foam as a drug form is described in the European Pharmacopoeia [Eur. Ph. 11.0] in the monograph “Foams, Medicated”. Medicated foams are preparations usually intended for application to the skin or mucous membranes and are formed, typically at the time of administration, by dispersion of a large volume of gas in a liquid. This liquid contains one or more active substances and generally a surfactant to help create the foam, together with various other excipients. The preparation is usually supplied in a pressurised multidose container equipped with a device consisting of a valve and a push button suitable for the delivery of the foam. The Pharmacopoeia monograph focuses mainly on the liquid foams applied topically, while the skilled person appreciates that carriers in the form of solid foams are increasingly used in pharmaceutical technology. The high gas content and the low volume of fluid allow for covering a large area of the skin with a small amount of fluid leading to acceptability of the foams. Moreover, the delicate structure will not cause irritation when spreading. A large amount of gas also causes low specific density of the foams, which allows them to float on the surface of the scalp. The contained ingredients (e.g. emulsifiers) increase permeation into the skin. Absorption into the skin usually takes places rapidly, while no oily or greasy film is left. Topical application of estetrol formulated as a foam increases the effectiveness of the action with the concomitant benefit of enlarged patient's comfort. This form of application increases the absorption of estetrol by the hair follicles and causes improvement in therapeutic effect. With foams, compliance is increased, which consequently also affects the effectiveness of treatment.

“Mousse” refers to a substance closely resembling a foam, but is commonly used to denote substances that are less aqueous.

“Sprays” are preparations of drugs in aqueous, alcoholic or glycerine containing media. They are applied to the mucosae or skin with an atomiser or nebuliser. “Spray” means that the drug-containing solution is filled in a device suitable for spraying the drug-containing solution and released in a mist with the help of pressure.

“Gels” are semi-solid systems in which liquids are solidified by gel skeleton formers. In a hydrogel, the liquid that forms the gel is water or an aqueous solution. The term “hydrogel” as used herein refers to an aqueous solution of active ingredients that is mainly solidified with macromolecular hydrophilic substances to form a gel. Macromolecular hydrophilic substances and thus polymeric materials swell when they come into contact with water, which, depending on the concentration, results in solutions with pseudoplastic flow behaviour or plastic structures that contain a necessary aqueous component. Thus, hydrophilic gels consist of water or aqueous solutions that are usually gelled with hydrophilic macromolecular compounds. Gels built up with hydrophilic macromolecular scaffolds are generally thixotropic (i.e. having a viscosity that decreases upon the application of pressure). In contrast to creams, gels are referred to as true single-phase systems. In preferred embodiments, the topical composition referred to herein may be a hydrogel, or is comprised in a hydrogel.

“Hydrogels” as envisaged herein may comprise any suitable polymer or combination of polymers, such as but not limited to hydrophilic polymers, acrylic acid, acrylamide, and 2-hydroxyethylmethacrylate. Examples of hydrogels include, but are not limited to synthetic hydrogels, stimuli-sensitive hydrogels, (poly) peptide based hydrogels, hybrid hydrogels, and DNA based hydrogels. Production methods for each of these hydrogel categories have been described in the art and are therefore known to a skilled person.

By means of illustration and not limitation, examples of synthetic hydrogels include double network hydrogels. Examples also include nanocomposite hydrogels. Stimuli-sensitive hydrogels are characterised by their capacity to undergo changes in swelling that may be mediated by external stimuli (e.g., pH, temperature, ionic strength, solvent type, electric field, magnetic field, light, and chelating species). Examples of stimuli-sensitive hydrogels include without limitation hydrogels formed from block co-polypeptides recombinant segments of natural structural proteins such as elastin, silk, silk-like, and elastin-like peptide blocks, and recombinant triblock copolymers of one or more polypeptide sequences.

The term “hybrid hydrogels” is used herein to indicate hydrogels comprising components from at least two distinct classes of molecules, such as for example synthetic polymers and biological macromolecules, interconnected either covalently or non-covalently.

In the (hydro)gel composition as described herein, the presence of some excipients such as propylene glycol, glycerol, Transcutol®, benzyl alcohol, and PEG400 can increase the solubility of estetrol. The use of these excipients in the gel composition allows estetrol to be soluble and completely available to permeate through the membrane or, similarly, through the stratum corneum. This contributes to an increase in the rate of diffusion across the membrane or, similarly, through the stratum corneum as predicted by Fick's law of diffusion, since a higher estetrol concentration gradient is produced by increasing estetrol solubility between the two sides of the membrane.

In embodiments wherein the topical composition is a hydrogel, it is preferred that said hydrogel comprises a permeation enhancer that enables permeation through the stratum corneum. More preferably, the hydrogel referred to herein is a hydrogel comprising of from about 0.02% to about 10% (w/w) of an estetrol component such as estetrol, preferably estetrol monohydrate. Preferably, the hydrogel referred to herein is a hydrogel comprising of from about 0.02% to about 5% (w/w) of an estetrol component, more preferably, the hydrogel referred to herein is a hydrogel comprising of from about 0.02% to about 2.5% (w/w) of an estetrol component, more preferably the hydrogel comprises of from about 0.03% to about 0.75% (w/w) of an estetrol component, preferably of from about 0.04% to about 0.5% (w/w) of an estetrol component, more preferably of from about 0.05% to about 0.25% (w/w) of an estetrol component, such as from about 0.08 to about 1.2% (w/w), from about 0.09% to about 1.1% (w/w), or from about 0.1% to about 1% (w/w), most preferably the hydrogel comprises of from about 0.06% (w/w) of an estetrol component.

Alternatively, the hydrogel referred to herein is a hydrogel comprising about 10% (w/w) or less of an estetrol component. Preferably, the hydrogel referred to herein is a hydrogel comprising about 5% (w/w) or less of an estetrol component, more preferably, the hydrogel referred to herein is a hydrogel comprising about 2.5% (w/w) or less of an estetrol component, more preferably the hydrogel comprises about 0.75% (w/w) or less of an estetrol component, preferably about 0.5% (w/w) or less of an estetrol component, more preferably about 0.25% (w/w) or less of an estetrol component, more preferably about 0.1% or less (w/w) of an estetrol component, most preferably about 0.08% or less (w/w) of an estetrol component.

In particular embodiments wherein the topical composition is a hydrogel, said hydrogel is characterised by a favourable release profile, e.g. when compared to other compositions such as, but not limited to creams. In preferred embodiments, the hydrogel described herein is characterised by a mean cumulative amount of estetrol component released of at least about 2.5 μg/cm2, at least about 5 μg/cm2, at least about 7 μg/cm2, at least about 10 μg/cm2, ate least about 15 μg/cm2, at least about 20 μg/cm2, at least about 25 μg/cm2, preferably at least about 50 μg/cm2, more preferably at least 100 μg/cm2, more preferably at least about 150 μg/cm2, more preferably at least about 200 μg/cm2 within 1 hour across an isopore membrane in a receptor solution of 40:30:30 v/v/v ethanol:PEG400:water. In alternative embodiments, the hydrogel described herein is characterised by a mean cumulative amount of estetrol component released of from about 25 μg/cm2 to about 200 μg/cm2 within 1 hour across an isopore membrane in a receptor solution of 40:30:30 v/v/v ethanol:PEG400:water. In preferred alternative embodiments, the hydrogel described herein is characterised by a mean cumulative amount of estetrol component released of from about 25 μg/cm2 to about 100 μg/cm2 within 1 hour across an isopore membrane in a receptor solution of 40:30:30 v/v/v ethanol:PEG400:water. In further preferred alternative embodiments, the hydrogel described herein is characterised by a mean cumulative amount of estetrol component released of from about 25 μg/cm2 to about 50 μg/cm2 within 1 hour across an isopore membrane in a receptor solution of 40:30:30 v/v/v ethanol:PEG400:water.

Preferably, the hydrogel described herein is characterised by a mean cumulative amount of estetrol component released of at least about 350 μg/cm2, preferably at least about 400 μg/cm2, more preferably at least about 450 μg/cm2 within 8 hours across an isopore membrane in a receptor solution of 40:30:30 v/v/v ethanol:PEG400:water.

Preferably, the hydrogel described herein is characterised by a mean % applied dose of the estetrol component into a receptor solution of 40:30:30 v/v/v ethanol:PEG400:water of at least about 15%, preferably at least about 20% after 1 hour and/or a mean % applied dose of the estetrol component into a receptor solution of 40:30:30 v/v/v ethanol:PEG400:water of at least about 40%, preferably at least about 50%, more preferably at least 80% after 8 hours. In alternative embodiments, the hydrogel described herein is characterised by a mean % applied dose of the estetrol component into a receptor solution of 40:30:30 v/v/v ethanol:PEG400:water of from about 15% to about 30% after 1 hour and/or a mean % applied dose of the estetrol component into a receptor solution of 40:30:30 v/v/v ethanol:PEG400:water of from about 45% to about 90% after 8 hours.

In highly preferred embodiments, the hydrogel described herein is characterised by an estetrol component release rate (i.e., slope) of from about 2.5 μg/cm2/√h, at least about 5 μg/cm2/√h, at least about 7 g/cm2/√h, at least about 10 μg/cm2/√h, ate least about 15 μg/cm2/√h, at least about 20 μg/cm2/h, at least about 25 μg/cm2/√h to about 50 μg/cm2/√h and a mean cumulative amount of estetrol component released of from about 50 μg/cm2 to about 100 μg/cm2 after 8 hours, and optionally a percentage of estetrol amount released of from about 75% to about 95% across an isopore membrane in receptor solution 40:30:30 v/v/v ethanol:PEG400:water. Yet more preferably, the hydrogel described herein is characterised by an estetrol component release rate (i.e., slope) of from about 30 μg/cm2/√h to about 35 μg/cm2/√h and a mean cumulative amount of estetrol component released of from about 75 μg/cm2 to about 90 μg/cm2 after 8 hours, and optionally a percentage of estetrol amount released of from about 85% to about 90% across an isopore membrane in receptor solution 40:30:30 v/v/v ethanol:PEG400:water. Most preferably, the hydrogel described herein is characterised by an estetrol component release rate (i.e., slope) of from about 33 μg/cm2/√h to about 34 μg/cm2/√h and a mean cumulative amount of estetrol component released of from about 82 μg/cm2 to about 83 μg/cm2 after 8 hours, and optionally a percentage of estetrol amount released of from about 86% to about 88% across an isopore membrane in receptor solution 40:30:30 v/v/v ethanol:PEG400:water.

In embodiments wherein the topical composition is a cream, said cream may be characterised by an estetrol component release rate (i.e., slope) of from about 2.5 μg/cm2/√h, at least about 5 μg/cm2/√h, at least about 7 μg/cm2/h, at least about 10 μg/cm2/√h, ate least about 15 μg/cm2/√h, at least about 20 μg/cm2/√h, or from 15 μg/cm2/√h to about 75 μg/cm2/√h across an isopore membrane in receptor solution 40:30:30 v/v/v ethanol:PEG400:water. Preferably, said cream may be characterised by an estetrol component release rate (i.e., slope) of from about 25 μg/cm2/√h to about 55 μg/cm2/√h across an isopore membrane in receptor solution 40:30:30 v/v/v ethanol:PEG400:water.

The cream may further be characterised by a mean cumulative amount of estetrol component released of from about 1 μg/cm2 to about 100 μg/cm2 after 1 hour across an isopore membrane in receptor solution 40:30:30 v/v/v ethanol:PEG400:water. Preferably, the cream may be characterised by a mean cumulative amount of estetrol component released of from about 5 μg/cm2 to about 80 μg/cm2 after 1 hour across an isopore membrane in receptor solution 40:30:30 v/v/v ethanol:PEG400:water. The cream may further be characterised by a mean cumulative amount of estetrol component released of from about 25 μg/cm2 to about 150 μg/cm2 after 8 hours across an isopore membrane in receptor solution 40:30:30 v/v/v ethanol:PEG400:water. Preferably, the cream may be characterised by a mean cumulative amount of estetrol component released of from about 50 μg/cm2 to about 120 μg/cm2 after 8 hours across an isopore membrane in receptor solution 40:30:30 v/v/v ethanol:PEG400:water.

In embodiments wherein the topical composition is a foam, the foam formulation may comprise one or more excipients selected from the group consisting of: butane, butylated hydroxytoluene (optionally butylated hydroxytoluene E321), cetyl alcohol, citric acid (optionally citric acid anhydrous), glycerol, isobutane, lactic acid, polysorbate 60, propane, purified water, stearyl alcohol, ethanol anhydrous. An exemplary foam formulation may comprise in addition to the estetrol component butane, butylated hydroxytoluene, cetyl alcohol, citric acid, glycerol, isobutane, lactic acid, polysorbate 60, propane, purified water, stearyl alcohol, and ethanol anhydrous. An alternative exemplary foam formulation may comprise in addition to the estetrol component ethanol anhydrous, purified water, butylated hydroxytoluene (E321), lactic acid, citric acid anhydrous, glycerol, cetyl alcohol, stearyl alcohol, polysorbate 60, propane, butane, and isobutane.

In embodiments wherein the topical composition is a solution, the solution may comprise ethanol (alcohol), propylene glycol, water, or any combination thereof as excipients.

In particular embodiments, the topical composition is formulated as a shampoo. Preferably, the topical composition is formulated as a shampoo for daily use.

As indicated above, any of the topical compositions disclosed herein may comprise a permeation enhancer. Preferably, the permeation enhancer comprises a molecule that enables permeation through the stratum corneum (i.e. a permeation enhancing molecule) and a solvent or solvent system. Optionally, the permeation enhancer is present in the topical composition disclosed herein in an amount of from about 0.5% to about 60% (w/w), preferably in an amount of from about 1% to about 50% (w/w), more preferably in an amount of from about 2.5% to about 45% (w/w), more preferably in an amount of from about 5% to about 40% (w/w), more preferably in an amount of from 10% to about 30% (w/w). Optionally, the permeation enhancer molecule is present in the topical composition disclosed herein in an amount of from about 0.1% to about 25% (w/w), preferably in an amount of from about 0.5% to about 15% (w/w), more preferably in an amount of from about 1% to about 10% (w/w), more preferably in an amount of from about 2.5% to about 7.5% (w/w), more preferably in an amount of from 3.5% to about 5% (w/w). Optionally, the solvent (system) is present in the topical composition disclosed herein in an amount of from about 1% to about 60% (w/w), preferably in an amount of from about 5% to about 50% (w/w), more preferably in an amount of from about 10% to about 40% (w/w), more preferably in an amount of from about 15% to about 30% (w/w), more preferably in an amount of from 18% to about 25% (w/w).

The permeation enhancer described herein is not particularly limiting for the invention and may therefore comprise or consist of a molecule selected from the group consisting of suberin, lignin, cutin, include dimethyl sulfoxide, ethanol, propylene glycol, glycerin, propylethylene glycols, urea, dimethyl acetamide, sodium lauryl sulfate, poloxamers, spans, tweens, lecithin, terpenes, and combinations thereof. Preferred permeation enhancing molecules in the context of the present invention include ethanol, ethers, benzyl alcohols, fatty acids and esters thereof, or any combination thereof. Particularly preferred permeation enhancer molecules in the context of the present invention include Transcutol®, benzyl alcohol, and any combination thereof. Benzyl alcohol (C₆H₅CH₂OH) may be interchangeably referred to in the art as “phenylmethanol”, “phenylcarbinol”, and “benzenemethanol”. Transcutol® is a commonly accepted trade name for 2-(2-ethoxyethoxy) ethanol, also annotated interchangeably in the art by diethylene glycol monoethyl ether (C₆H₁₄O₃). Optionally, the topical composition described herein comprises as permeation enhancer of from about 1% to about 20% Transcutol® (w/w), preferably of from about 2% to about 10% Transcutol® (w/w), more preferably of from about 3% to about 8% Transcutol® (w/w), most preferably of from about 4% to about 6% Transcutol® (w/w).

The permeation enhancers of the present invention comprise a permeation enhancer molecule and a solvent or solvent system. Preferred solvents of the permeation enhancer include but are not limited to polyethylene glycols (PEG), propylene glycol (PG), and combinations thereof. “Polyethylene glycol” may be interchangeably indicated by terms such as but not limited to polyethylene oxide or poly(oxyethylene), poly(ethylene oxide), and polyoxyethylene have been described in detail in the art and are therefore known to a skilled person, who appreciates the polyethylene glycol is characterised by the chemical formula H—(O—CH₂—CH₂)ₙ—OH wherein n is an integer. Polyethylene glycols are polyether compounds derived from petroleum. Preferred PEGs are PEGs characterised by a molecular weight of from between about 150 g/mol to about 5000 g/mol, more preferably of from between about 200 g/mol to about 2500 g/mol, yet more preferably between about 250 g/mol to about 1000 g/mol, most preferably between about 300 g/mol to about 600 g/mol. Therefore, the PEG referred to herein may be selected from the group consisting of: PEG200, PEG300, PEG400, PEG500, PEG600, and any combination thereof. Most preferably, the permeation enhancer comprises as solvent or part of the solvent a PEG having a molecular weight of about 400 g/mol such as but not limited to PEG400. Propylene glycol, commonly annotated in the art as propane-1,2-diol, α-propylene glycol, 1,2-propanediol, 1,2-dihydroxypropane. Propylene glycol is characterised by the chemical formula CH₃CH(OH)CH₂OH.

Preferably, the permeation enhancer comprises of from about 5% to about 35% (w/w) PEG, preferably PEG400, and/or of from about 10% to about 35% (w/w) PG. More preferably, the permeation enhancer comprises of from about 10% to about 30% (w/w) PEG, preferably PEG400, and/or of from about 12% to about 30% (w/w) PG. More preferably, the permeation enhancer comprises of from about 12% to about 25% (w/w) PEG, preferably PEG400, and/or of from about 15% to about 25% (w/w) PG. More preferably, the permeation enhancer comprises of from about 14% to about 23% (w/w) PEG, preferably PEG400, and/or of from about 16% to about 23% (w/w) PG. More preferably, the permeation enhancer comprises of from about 16% to about 22% (w/w) PEG, preferably PEG400, such as from about 18% to about 22% (w/w) PEG or from about 16% to about 20% (w/w) PEG, and/or from about 18% to about 22% (w/w) PG.

Optionally, the topical composition comprises benzyl alcohol. The term “benzyl alcohol” is to be interpreted according to its common interpretation in the art and therefore indicates an aromatic alcohol characterised by the chemical formula C₆H₅CH₂OH. If present, the amount of benzyl alcohol in the topical composition is not particularly limiting. However, preferably the amount of benzyl alcohol is of from about 0.1% to about 10% (w/w), more preferably of from about 0.5% to about 5% (w/w), more preferably of from about 1% to about 3% (w/w), yet more preferably of from about 1.5% to about 2.5% (w/w).

Optionally, the topical composition comprises a thickener. A “thickener” may be alternatively indicated by terms such as but not limited to “thickening agent” and “viscosity agent”. “thickener” as used in the context of the present invention indicates any substance or molecule that upon addition to a liquid or semi-solid composition increases the viscosity and/or texture of said composition. Hence, if present, the thickener may be selected from the group consisting of: carboxylic acid polymers, crosslinked polyacrylate polymers, polyacrylamide polymers, polysaccharides, diblock polymers, triblock polymers, gums, and any combination thereof. In embodiments wherein the thickener is or comprises a polysaccharide, said polysaccharide may be selected from the group consisting of: cellulose, cellulose derivatives, carboxymethyl cellulose, cellulose acetate propionate carboxylate, hydroxyethyl cellulose, hydroxyethyl ethylcellulose, hydroxypropyl cellulose, hydroxypropyl methylcellulose, methyl hydroxyethyl cellulose, hydroxyalkylated cellulose, lignin, cutin, suberin, microcrystalline cellulose, sodium cellulose sulfate, scleroglucans, and any combination thereof. Suitable gums that may act as thickener include acacia, agar, algin, alginic acid, cetyl alcohol, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and any combinations thereof.

In certain embodiments, the topical composition described herein comprises a thickener as described herein in an amount of from about 0.1% to about 25% (w/w), preferably in an amount of from about 0.3% to about 20% (w/w), more preferably in an amount of from about 0.4% to about 15%, more preferably in an amount of from about 0.5 to about 10% (w/w), more preferably in an amount of from about 0.75% to about 5% (w/w). Preferred thickeners in the context of the present invention include but are not limited to thickeners selected from the group consisting of: hydroxyethyl cellulose (HEC), carboxymethyl cellulose (CMC), high molecular weight cross-linked acrylic based polymer, non-ionic triblock copolymer, or any combination thereof. More preferred thickeners in the context of the present invention include but are not limited to hydroxyethyl celluloses, high molecular weight cross-linked acrylic based polymers, non-ionic triblock copolymer, and any combination thereof. A preferred high molecular weight cross-linked acrylic based polymer is Carbomer, interchangeably indicated throughout the art by the trade name Carbopol®. Both Carbopol® homopolymers (i.e. acrylic acid crosslinked with allyl sucrose or allyl pentaerythritol), Carbopol® copolymers (acrylic acid and C10-C30 alkyl acrylate crosslinked with allyl pentaerythritol), Carbopol® interpolymers (carbomer homopolymer or copolymer that contains a block copolymer of polyethylene glycol and a long chain alkyl acid ester) are envisaged. A particularly preferred Carbopol® in the context of the present invention is Carbopol® 980, alternatively indicated throughout the art as “Carbomer Homopolymer Type C USP NF”, which is an homopolymer of acrylic acid crosslinked with allyl sucrose or allyl pentaerythritol in cosolvent of cyclohexane and ethyl acetate having a viscosity of 40000 to 60000 cP. A preferred hydroxyethyl cellulose is HEC250 HHX. A preferred non-ionic triblock copolymer is a non-ionic triblock copolymer having a molecular mass of from about 1800 g/mol and about 4000 g/mol and a polyoxyethylene content of from about 70% to about 80%. Highly preferred non-ionic triblock copolymers include poloxamer 188, poloxamer 407, or combinations thereof.

In certain embodiments, the thickener is or comprises HEC and is present in the topical composition in an amount of from about 0.1% to about 10% (w/w), preferably in an amount of from 0.2% to about 7.5% (w/w), more preferably in an amount of from about 0.5% to about 5% (w/w), more preferably in an amount of from about 0.75% to about 2.5% (w/w), most preferably in an amount of from about 1% to about 2% (w/w). In certain embodiments, the thickener is or comprises Carbopol® and is present in the topical composition in an amount of from about 0.1% to about 10% (w/w), preferably in an amount of from 0.2% to about 7.5% (w/w), more preferably in an amount of from about 0.5% to about 5% (w/w), more preferably in an amount of from about 0.75% to about 2.5% (w/w), most preferably in an amount of from about 0.75% to about 1.5% (w/w). In alternative embodiments, Carbopol® is present in the topical composition in an amount of from about 0.1% to about 1% (w/w). In certain embodiments, the thickener is or comprises poloxamer 188 and is present in the topical composition in an amount of from about 0.1% to about 30% (w/w), preferably in an amount of from about 1% to about 15% (w/w), more preferably in an amount of from about 2.5% to about 10% (w/w). In certain embodiments, the thickener is or comprises poloxamer 407 and is present in the topical composition in an amount of from about 0.1% to about 30% (w/w), preferably in an amount of from about 5% to about 25% (w/w), more preferably in an amount of from about 10% to about 20% (w/w).

In certain embodiments, the thickener is or comprises CMC and is present in the topical composition in an amount of from about 0.1% to about 10% (w/w), preferably in an amount of from 0.2% to about 7.5% (w/w), more preferably in an amount of from about 0.5% to about 5% (w/w), more preferably in an amount of from about 0.75% to about 2.5% (w/w), most preferably in an amount of from about 1% to about 2% (w/w) or about 1.5% (w/w).

Optionally, the topical composition comprises a preservative. In particular embodiments, the topical composition comprises a preservative in an amount of from 0.5% to 20% (w/w), preferably in an amount of from 1% to about 10% (w/w), more preferably in an amount of from about 1% to about 3% (w/w). The exact preservative is not particularly limiting for the invention and may therefore be selected from the group consisting of: lysozyme, nisin, quaternary ammonium preservatives, parabens, phenoxyethanol, benzyl alcohol, chlorobutanol, phenol, sorbic acid, thimerosal, natural preservatives, and any combination thereof. A preferred preservative in the context of the invention is benzyl alcohol.

Optionally, the topical composition (which may be a hydrogel) comprises an emollient. “Emollient” as used throughout the present disclosure refers to a material useful for preventing and/or treating a dryness of the skin, as well as providing an extra protection of the skin. The particular emollient is not particularly limiting for the invention and may therefore be selected from the group consisting of: glycerol, acetyl alcohols, stearyl alcohol, stearic acid, isopropyl palmitate, squalene, lanolin, glycerine, petrolatum, petroleum, and any combination thereof. A particularly preferred emollient in the context of the present invention is glycerol. The emollient may be present in the topical composition in an amount of from about 2.5% to about 30% (w/w), preferably in an amount of from about 5% to about 25%, more preferably in an amount of from 7.5% to about 20% (w/w), more preferably in an amount of from about 8% to about 12% (w/w). In further embodiments, the emollient may be glycerol that is present in the topical composition in an amount of from about 2.5% to about 30% (w/w), preferably in an amount of from about 5% to about 25% (w/w), more preferably in an amount of from 7.5% to about 20% (w/w), more preferably in an amount of from about 8% to about 12% (w/w), most preferably in an amount of about 10% (w/w).

As described in detail throughout the present disclosure, any of the topical compositions described herein may comprise a certain amount of components (i.e. pharmaceutically active agents and/or excipients) in addition to the estetrol component. In any of the embodiments of the invention, a solvent can be added to arrive at certain concentrations of said components. In further embodiments, an aqueous solution is used to complement the topical composition. The term “aqueous solution” refers to any solution comprising water or in which the solvent is water. Additionally, “aqueous solution” is used to describe solutions displaying commonalities to water or watery solutions, not limited to characteristics such as appearance, smell, colour, taste, viscosity, pH, absorbance, or physical state under particular temperatures. In such embodiments, the aqueous solution may be water. In alternative further embodiments, a non-aqueous solution is used to complement the topical composition. In yet alternative embodiments, a mixture of a non-aqueous solution and an aqueous solution is used to complement the topical composition.

As defined herein, the pH of a topical composition, solution, or formulation may be measured using various methods as known to a person skilled in the art. pH indicators may be used that discolour by uptake or release of H+-ions, wherein their resulting colour is indicative for a certain pH value. Alternatively, pH meters may be used that measure the difference in electrical potential between a pH electrode and a reference electrode. The difference in electrical potential relates to the acidity or pH of the solution.

Considering the above, an exemplary topical composition according to the invention comprises in addition to the estetrol component in any one of the concentrations listed herein:
from about 0.1% to about 60% (w/w) of a permeation enhancer;
from about 0.3% to about 20% (w/w) of a thickener;
optionally a preservative and/or an emollient;
water up to 100% (w/w).

In certain embodiments, the topical composition consists essentially of, or consists of a permeation enhancer, a thickener, a preservative, and an emollient (in addition to the estetrol component). In preferred embodiments, the topical composition according to the invention comprises in addition to the estetrol component:
from about 0.1% to about 10% (w/w) of a permeation enhancer;
from about 10% to about 60% (w/w) of a permeation enhancer solvent;
from about 0.3% to about 20% (w/w) of a thickener;
optionally a preservative and/or an emollient;
water up to 100% (w/w).

In yet further preferred embodiments, the topical composition according to the invention comprises in addition to the estetrol component in any one of the concentrations listed herein:
from about 1% to about 7.5% (w/w) of a permeation enhancer;
from about 15% to about 45% (w/w) of a permeation enhancer solvent;
from about 0.3% to about 20% (w/w) of a thickener;
optionally a preservative and/or an emollient;
water up to 100% (w/w).

In a particular embodiment, the topical composition comprises, consists essentially of, or consists of, in addition to the estetrol component in any one of the concentrations listed herein:
from about 8% to about 40%, preferably from about 16% to about 20%, most preferably about 18% (w/w) PEG400;
from about 9% to about 44%, preferably from about 18% to about 22%, most preferably about 20% (w/w) PG;
from about 4% to about 24%, preferably from about 8% to about 12%, most preferably about 10% (w/w) glycerol;
from about 0.5% to about 4%, preferably from about 1% to about 2%, most preferably about 1.5% (w/w) HEC; and
from about 0.75% to about 1.25%, preferably from about 1.5% to about 2.5%, most preferably about 2% (w/w) benzyl alcohol.

In a particular embodiment, the topical composition comprises, consists essentially of, or consists of, in addition to the estetrol component in any one of the concentrations listed herein:
from about 9% to about 44%, preferably from about 18 to about 22%, most preferably about 20% (w/w) PEG400;
from about 0.01% to about 2%, preferably from about 0.1% to about 1%, most preferably about 0.5% (w/w) Carbopol®; and
from about 2% to about 12%, preferably from about 4% to about 6%, most preferably about 5% (w/w) Transcutol®.

The terms “formulation” or “composition” may be used interchangeably herein. In any of the embodiments concerning the topical compositions described herein, it is evident that said compositions may comprise one or more pharmaceutically or cosmetically acceptable carriers (i.e. excipients) that are not described in detail throughout the present disclosure. The term “pharmaceutically acceptable” as used herein is consistent with the art and means compatible with the other ingredients of a pharmaceutical or cosmetic topical composition and not deleterious to the recipient thereof. In a particularly preferred embodiment of the invention the topical composition according to invention is designed for daily administration, i.e. it represents a daily dosage unit. The excipients that may be used in the topical composition is not particularly limited and may therefore be one or more excipients selected from the group consisting of: an active pharmaceutical ingredient excipients, binder excipients, carrier excipients, co-processed excipients, coating system excipients, controlled release excipients, diluent excipients, disintegrant excipients, dry powder inhalation excipients, effervescent system excipients, emulsifier excipients, lipid excipients, lubricant excipients, modified release excipients, penetration enhancer excipients, permeation enhancer excipients, pH modifier excipients, plasticizer excipients, preservative excipients, preservative excipients, solubilizer excipients, solvent excipients, sustained release excipients, sweetener excipients, taste making excipients, thickener excipients, viscosity modifier excipients, filler excipients, compaction excipients, dry granulation excipients, hot melt extrusion excipients, wet granulation excipients, rapid release agent excipients, increased bioavailability excipients, dispersion excipients, solubility enhancement excipients, stabilizer excipients, capsule filling excipients, or any combination hereof. A skilled person is aware that use of such media and agents for pharmaceutical active substances is common practice and incorporation of these excipients is hence well known in the art. It is evident that all of the used ingredients should be non-toxic in the concentration contained in the final topical composition and should not negatively interfere with the activity of the estetrol component, said estetrol component preferably being present in the topical composition as the predominant pharmaceutically active ingredient. In certain embodiments, more than one excipient which a skilled person would classify as belonging to the same group of excipients is added to the topical composition. In further embodiments, more than one excipient wherein the different excipients belong to different groups is added to the topical composition. In certain embodiments, the excipients may fulfil more than one function and/or be classified by a skilled person as belonging to different groups or classes of excipients.

Optionally, the topical compositions envisaged by the present invention may comprise further skin active components capable of providing a skin care benefit and/or hair care benefit. The skin care benefit may include but is not limited to benefits related to cosmetic appearance of the skin. The hair care benefit may include but is not limited to benefits related to cosmetic appearance of the hair. The further skin active component may provide an immediate and short lived (i.e. acute) benefit, and/or a long term and long lasting (i.e. chronic) benefit.

Optionally, the topical compositions envisaged by the present invention may comprise at least one further pharmaceutically active ingredient in addition to the estetrol component. In particular embodiments, the at least one further pharmaceutically active ingredient is selected from the group consisting of: anti-inflammatory agents, analgesic agents, and anti-infective agents. The anti-inflammatory ingredient may be a steroidal anti-inflammatory ingredient, a nonsteroidal anti-inflammatory ingredient, or a combination thereof.

In certain embodiments, the further pharmaceutically active ingredient may also have the property of reducing female or male (pattern) hair loss. Non-limiting examples of such ingredients include without limitation: minoxidil, finasteride, spironolactone, and dutasteride.

The topical composition as described herein may be part of, i.e., comprised in any means that is suitable for application to the scalp of a subject. Alternatively, the topical composition as described herein may be applied to any means that is suitable for application to the scalp of a subject by said subject, another subject, or a skilled medical practitioner prior to application of the composition to the scalp of the subject (i.e. the composition may be used in conjunction with the means for application to the scalp).

Alternatively, the topical composition may be comprised in any other suitable means for application to the scalp of a subject, and may therefore be comprised in means such as but not limited to bandages, band aids, patches, and plasters.

A skilled person appreciates that the topical compositions and formulations described herein may be used as both a cosmetic composition (i.e. for cosmetic purposes) and a therapeutic composition (i.e. for therapeutic purposes). A skilled person appreciates that cosmetic applications have the goal to improve the physical appearance of the subject wherein the subject is considered to be in good health. In contrast, therapeutic applications include situations wherein the hair loss of the subject is caused by, or related to, a medical aberrant condition. By means of illustration and not limitation, the medical aberrant condition may be hormone dysregulation, may be related to certain medical treatments such as but not limited to chemotherapy, or may be occurring the context of an autoimmune disorder.

### Hair Loss

The term “hair loss” as used herein relates to any kind of hair loss in a subject (female or male), preferably an adult subject, more preferably a male adult subject. “Hair loss” as used herein broadly refers to any kind of alopecia. Thus, “hair loss” as used herein encompasses the clinical conditions better known as androgenetic alopecia (AGA), or alopecia areata (AA), fibrosing alopecia, diffuse alopecia, scarring alopecia, and universal alopecia. Hence, the topical composition described herein may be used for treating alopecia, in particular androgenetic alopecia. Androgenetic alopecia has been described in great detail in the art at numerous occasions and is therefore known to a person skilled in the art (for example in Chin et al., Androgenetic alopecia, StatPearls, 2022). Male androgenetic alopecia (interchangeably indicated by the term “pattern hair loss”) is a hair loss condition that mainly affects the top and front of the scalp. Male-pattern hair loss is generally described as a receding front hair line, hair loss on the crown of the scalp, or a combination thereof. Female androgenetic alopecia is a hair condition that mainly affect the crown and the frontal areas of the scalp in a generally more diffuse manner when compared to male androgenetic alopecia.

The present invention thus provides in an aspect a topical composition comprising an estetrol component for use in preventing or treating hair loss as defined herein elsewhere. Alternatively worded, the present invention envisages the use of a topical composition comprising an estetrol component for preventing or treating hair loss, and improvement in hair texture, quality and appearance. Yet alternatively worded, the present invention envisages use of an estetrol component (or a composition comprising an estetrol component) for the manufacture of a medicament for preventing or treating hair loss by topical administration. Finally, the invention further provides a method of treatment for preventing or reducing hair loss comprising administration of a composition comprising topical administration of an estetrol component to a subject. Numerous methods and approaches to measure and/or monitor hair loss have been described in the art (reviewed in e.g. Dhurat and Saraogi, Int J Trichology, 2009), such as target area hair count (“TAHC”) and target area hair width (“TAHW”). The terms target area hair width and target area hair thickness may be used interchangeably herein and throughout the art.

In certain embodiments, use of the topical composition results in a decrease in TAHC and/or decrease in TAHW in a (female or male) subject that is less than the decrease in respectively TAHC and/or TAHW in said (female or male) subject prior to use of the topical composition subject of the invention. Optionally, the decrease is at least 10% less, preferably at least 20% less, preferably at least 30% less, preferably at least 40% less, preferably at least 50% less, than the decrease in said subject prior to the use of the topical composition subject of the invention.

Terms such as “subject”, “patient”, “individual” may be used interchangeably herein and refer to human subjects. Preferred subjects are male human subjects. In certain embodiments, the subject is considered to be a male subject of adult age (i.e. at least 18 years old). In preferred embodiments, the male subject is a subject who is or is considered to be a post puberty male subject. In preferred embodiments, the subject is a male subject diagnosed to be, or considered to be experiencing hair loss, balding, or hairline regression. In yet further preferred embodiments, the male subject is a male adult subject of from 19 to 49 years old.

Alternatively, the subject is a female subject. Optionally, the subject is a female subject of adult age (i.e. at least 18 years old). In certain embodiments, the subject is a female menopausal, perimenopausal, and/or postmenopausal subject. In certain embodiments, the subject is a menopausal, perimenopausal, or postmenopausal subject having an estradiol level of less than 100 pg/ml, preferably less than 50 pg/ml, preferably less than 30 pg/ml, more preferably less than 20 pg/ml, more preferably less than 20 pg/ml, most preferably less than 10 pg/ml. In alternative embodiments, the subject is a menopausal, perimenopausal, or postmenopausal subject that is characterised by having follicle-stimulating hormone concentrations of at least 20 milli-international units per millilitre (mlU/ml), preferably at least 25 mlU/ml, more preferably at least 30 mlU/ml, more preferably at least 35 mlU/ml, most preferably at least 40 mlU/ml.

“Menopausal subjects”, used interchangeably in the art with “post-menopausal subjects” or “climacteric subjects” are female subjects that that have not had menstrual bleeding for a year which is accompanied by a decrease or discontinuation in hormone production by the ovaries (such as estradiol). Alternatively worded, “menopause” may be described as a biological condition characterised by impairment or cessation of ovarian primary function. Menopause may be accompanied by a broad range of clinical symptoms which are variable in severity such as but not limited to vasomotor dysfunction, vaginal dryness, mood changes, sleep disturbances, urinary incontinence, cognitive changes, somatic complaints, and sexual dysfunction. Methodologies to diagnose menopause have been described in the art and are therefore known to a person skilled in the art (Nelson, Menopause, Lancet, 2008).

“Perimenopause” refers to a period of life which begins approximately three to four years prior to menopause and ends one year after the final menstrual period, and is characterised by persistent irregular menstrual cycles, extreme fluctuations in hormonal levels, frequent anovulation and the appearance of vasomotor symptoms (Harlow et al., Executive summary of the Stages of Reproductive Aging Workshop+10: addressing the unfinished agenda of staging reproductive aging, Menopause, 2012). The term “post menopause” or “postmenopausal” is indicative for female subjects that are characterised by a permanent cessation of menstrual periods. This permanent cessation is determined retrospectively after an observation of 12 months of amenorrhea without any other obvious pathological or physiological cause. The term “post menopause” also includes menopause as the consequence of premature ovarian failure, surgery (ovariectomy for example), chemotherapy or radiotherapy for cancer, and certain diseases (for example, infections or hypothyroidism).

In particular embodiments wherein the subject is a male subject, the male pattern hair loss is classified as, or considered to be, male pattern baldness selected from stages 1 to 7 on the Hamilton-Norwood scale, preferably selected from stages 3 to 7 on the Hamilton-Norwood scale, more preferably stage 5, 6, or 7 on the Hamilton-Norwood scale. A person skilled in the art is familiar with the Hamilton-Norwood scale and appreciates that the scale is a widely used instrument to categorize hair loss (i.e. male androgenetic alopecia in men) (Gupta and Mysore, J Cutan Aesthet Surg, 2016).

While certain preferred forms of alopecia are described above, it is to be understood that the present invention envisages treatment of any form of alopecia. Non-limiting examples of particular forms of alopecia include without limitation: acne keloidalis, alopecia areata, alopecia totalis, alopecia universalis, anagen effluvium, androgenetic alopecia, Brocq's alopecia, central centrifugal cicatricial alopecia, cicatricial alopecia, congenital alopecia, congenital atrichia, diffuse alopecia areata, discoid lupus erythematosus, dissecting cellulitis, “end-stage” cicatricial alopecia, folliculitis decalvans, frontal fibrosing alopecia, hair-shaft abnormalities, hyperandrogenism, hypotrichosis, inherited alopecia, lichen planus, lichen planopilaris, lipedematous alopecia, male pattern hair loss, non-cicatricial alopecias, psoriasis, psoriasiform alopecia, pressure alopecia, seborrheic dermatitis psoriasis, telogen effluvium, temporal triangular alopecia, tinea capitis, TNF-a inhibitor-induced psoriasiform alopecia, traction alopecia, trichorrhexis nodosa, trichotillomania, and tufted folliculitis.

The present inventors have unexpectedly found that estetrol has a positive effect on the proliferation and functioning of keratinocytes.

The term “keratinocytes” encompasses a type of cells found in the epidermis and in the epithelial hair follicle cells encompassing bulge keratinocytes, outer root sheath (ORS) keratinocytes and hair matrix keratinocytes. In the outer layer of the skin, keratinocytes constitute about 90% of cells. The term “increasing keratinocyte function” is meant to include both increased proliferation of keratinocytes in the epidermis and hair follicles as well as increasing, improving or restoring the function of keratinocytes in the epidermis and hair follicles. Said function includes, but is not limited to, skin thickness and hydration, providing an adequate skin barrier and ensuring proper wound healing, promoting growth of hair follicle cells during anagen and epithelial-mesenchymal interaction, i.e. interaction with mesenchymal cells such as fibroblasts of the hair follicle, including dermal papilla cells.

Keratinocytes also play a role in oxidation and inflammation. Elevation in ROS production (generated amongst others in keratinocytes) and oxidative stress increase damage to DNA, proteins and lipids, and can lead to premature skin aging and hair loss. Chronic low-grade inflammation damages the skin by increasing the expression of proinflammatory cytokines by keratinocytes leading to detrimental changes and premature skin aging and hair loss.

Furthermore, skin pigmentation is influenced by keratinocytes as well as melanocytes, whose primary function is the production of the melanin pigment, are surrounded by keratinocytes, to which they transfer their melanin pigment. Due to the tight interaction and communication between melanocytes and keratinocytes, a single melanocyte can be surrounded by 36 keratinocytes, influencing keratinocytes e.g. by estetrol also influences melanocytes and hence pigmentation. The term “melanin” as used in the present disclosure refers to a group of natural pigments produced by melanocytes that are localized in the epidermis. Different types of melanin have been described in the art (e.g. in Wei et al., Unraveling the structure and function of melanin through synthesis, J Am Chem Soc, 2021). “Melanogenesis” is commonly used in the art and relates to the process of melanin production. Melanogenesis is for example upregulated upon exposure of the skin to UV radiation and results in a darkening of the skin tone.

The term “reactive oxygen species”, commonly abbreviated as “ROS” is an umbrella term for molecules formed from oxygen (O₂). Illustrative examples include peroxides, superoxides, hydroxyl radicals, singlet oxygens, and alpha oxygens. Reactive Oxygen Species are, among others, produced in fibroblasts and keratinocytes. Both the dermis and epidermis can protect the skin against oxidation, while the epidermis contains the highest concentration of antioxidants. ROS is a negative or aggravating factor in terms of female pattern hair loss due to damage caused to the hair follicles. Reducing ROS is hence important for avoiding female pattern hair loss.

Keratinocytes are also an important cell type for promoting wound healing, as they are responsible for the wound closure and the re-epithelialization but also for modulating inflammation in the wound next to immune cells. An imbalance in keratinocyte function can lead to imbalanced inflammation.

Additionally, the inventors have found that estetrol also positively influences the proliferation and functioning of fibroblasts.

The term “fibroblasts” encompasses a type of cells found in the dermis and in the mesenchymal hair follicle cells encompassing dermal papilla cells. Such fibroblast also include dermal papilla cells in the hair follicle which, although virtually not proliferating, are growing in number through interactions with surrounding keratinocytes due to epithelial mesenchymal interactions (EMI). Dermal fibroblasts are the main cell type present in skin connective tissue (dermis). Fibroblasts interact with epidermal cells during hair development and in interfollicular skin.

### Treatment and Prevention

The terms “treatment” or “treat” are to be interpreted as both the therapeutic treatment of a symptom, disease or condition that has already developed, leading to (clinical) manifestations, as well as prophylactic or preventive measures, wherein the goal of the treatment is to prevent, lessen, or reduce the chances of incidence of an undesired affliction, such as to prevent occurrence, development and progression of symptoms, (clinical) conditions or diseases related to hair loss. Therefore, a further aspect of the invention is directed to an effective amount of an estetrol component for use in the prevention of hair loss in a subject. Beneficial or desired clinical results may include, without limitation, alleviation of one or more symptoms, improvement of one or more biological markers, diminishment of extent of disease, stabilized (i.e. not worsening) state of disease, delay or slowing of disease progression, amelioration or palliation of the disease state, and the like.

“Prevention” or “prevent” as used in the context of the invention refers to an aversion of manifestation of a condition or disease image in a subject, i.e. the establishment of preventive measures or prophylactic measures. Preventive treatment refers to treatments wherein the object is to avoid a subject's body or an element thereof to show (worsening of) symptoms of an undesired physiological change.

As used herein, the terms “therapeutic treatment” or “therapy” and the like, refer to treatments wherein the aim is to change a subjects body or a part of a subjects body from an undesired physiological state, disease or disorder which is caused by aging, to a desired state, such as a less severe state (e.g. amelioration, or even back to its normal, healthy state (e.g. restoring the health, the physical integrity and the physical well-being of a subject), to keep it (i.e., not worsening) at said undesired physiological status (e.g. stabilization), or slow down progression to a more severe or worse state compared to said undesired physiological change or disorder. Measurable lessening includes any statistically significant decline in a measurable marker or symptom. Statistically significant as used herein refers to p values below 0.05, which is a commonly accepted cut-off score in statistical analysis as a skilled person appreciates. “Treatment” encompasses both curative treatments and treatments directed to reduce symptoms and/or slow progression and/or stabilize the condition or disease.

A skilled person is aware that in order to achieve an effective therapeutic treatment, a therapeutically effective dose needs to be administered to said subject. Therefore, in the context of the present disclosure “an effective amount” refers to an amount necessary to obtain a physiological effect. The physiological effect may be achieved by a single dose or by multiple doses. A “therapeutically effective amount” or “therapeutically effective dose” indicates an amount of estetrol component that when administered brings about a clinical positive response with respect to treatment of a subject afflicted by hair loss. Similarly, a “prophylactically effective amount” or “prophylactically effective dose” refers to an amount of estetrol component that inhibits or delays the onset of (clinical) manifestation of hair loss. A skilled person is aware that terms such as “quantity”, “amount” and “level” are synonyms and have a well-defined meaning in the art and appreciates that these may particularly refer to an absolute quantification of an estetrol component which is considered an effective amount for the applications described herein, or to a relative quantification of the estetrol component, such as for example a concentration of estetrol component in function of the subject's bodyweight. Suitable values or ranges of values may be obtained from one single subject or from a group of subjects (i.e. at least two subjects).

It is emphasised that while any of the herein disclosed values and ranges of the estetrol components are suitable for the different medical indications or purposes, a skilled person is aware that certain individuals may experience yet improved benefits from the estetrol component treatment by further optimisation optimal dose of said component by considering a wide range of parameters including but by no means limited to the nature and degree of the condition or disease to be treated, gender of the subject, subject age, body weight, other medical indications, nutrition, mode of administration, metabolic state, interference or influence by or efficacy of other pharmaceutically active ingredients, etc. Furthermore, each individual may have a certain intrinsic degree of responsiveness to the estetrol component that is used.

### Administration

The composition subject of the invention is particularly configured for topical administration, i.e. local application to the scalp of a subject.

Alternative to medical treatment methods or medical uses as defined herein, the invention equally envisages cosmetic products or cosmetic methods to prevent or reduce hair loss comprising a step of topical administration of a composition comprising an estetrol component. Alternatively worded, the invention equally envisages cosmetic use of a topical composition comprising an estetrol component for preventing or reducing hair loss. A skilled person appreciates that “cosmetic use” implies a “non-medical, non-therapeutic use”, thus a use that does not aim to ameliorate, prevent or treat a clinical pathology.

The present therapeutic or cosmetic therapy usually employs continuous administration of the estetrol component during a period of at least 10 days, preferably of at least 20 days.

The terms “continuous” and “continuously” as used herein, means that the components are administered at relatively regular intervals, with no (therapeutically) significant interruptions. Naturally, minor interruptions may occur that do not affect the overall effectiveness of the present method, and indeed such aberrations are encompassed by the present invention. In a preferred embodiment, and more arithmetically, the administration regimen is deemed to be continuous if the longest interval between two subsequent administrations is not more than 3.5 times as long as the average interval. Even more preferably said longest interval is not more than 2.5 times, most preferably not more than 1.5 times as long as the average interval.

A skilled person therefore appreciates that the estetrol component may be administered in conjunction with any other cosmetic or pharmaceutically active agents for alleviating or preventing hair loss known in the art.

In the therapeutic or cosmetic methods according to the present invention the estetrol component is suitably administered continuously during a period of at least 10 days.

The invention may suitably be reduced to practice in the form of a variety of administration methods that are known to the person skilled in the art. Amongst these methods are the methods making use of monophasic preparations, which contain dosage units with a constant amount of the estetrol component.

In a particularly preferred embodiment of the invention the topical composition according to invention is designed for daily administration, i.e. it represents a topical composition that is to be applied topically, e.g. to the scalp of a subject, once in about 24 hours.

The topical composition may be applied by pouring, dropping, spraying, rubbing, or by any other appropriate means. Optionally, the composition is administered once to the scalp. Alternatively, the composition is administered at multiple points in time to the scalp, preferably at substantially regularly interspersed time points. In certain embodiments, the composition is administered daily to the scalp. Optionally, the topical composition is administered to and maintained on (i.e. not removed from the scalp) for a prolonged period of time corresponding to at least 30 minutes, preferably at least 1 hour, more preferably at least 2 hours, more preferably at least 4 hours, more preferably at least 8 hours, more preferably at least 1 day, more preferably at least 1 week, more preferably at least 1 month. In such embodiments, it is understood that the scalp is continuously exposed to the composition for the indicated amount of time.

In certain embodiments, the estetrol component is comprised in an immediate release topical composition. In alternative embodiments, the estetrol component is comprised in a delayed release, sustained release, or controlled release topical composition. The terms “immediate release”, “delayed release”, and “sustained-release” or “controlled release” are clear to a person skilled in the art and are indicative for the release profile of a topical composition. In immediate release the topical composition is about immediately released from a dosage unit to a body of a subject or patient. In delayed release, composition is delivered in the body with a delay after administration. In sustained release or controlled release dosage units, the composition is designed to release the active pharmaceutical ingredient(s) at a predetermined rate in order to maintain a constant drug concentration for a specific period of time. The release profile of a dosage unit can be assessed as described in the major pharmacopeias. For example, immediate release is defined by the European Medicines Agency as dissolution of at least 75% of the active substance within 45 minutes (European Pharmacopeia (Ph. Eur.) 11^{th} edition). However, it is in addition trivial to a person skilled in the art that suitable tests and time windows may vary depending on therapeutic ranges, solubility and permeability factors of the drug substance.

The topical compositions envisaged by the present invention may comprise further skin active components capable of providing a skin care benefit. The skin care benefit may include but is not limited to benefits related to cosmetic appearance of the skin. The further skin active component may provide an immediate and short lived (i.e. acute) benefit, and/or a long term and long lasting (i.e. chronic) benefit.

The topical compositions envisaged by the present invention may comprise further hair loss preventing or hair care promoting active components capable of providing a hair care benefit known in the art. The hair care benefit may include but is not limited to benefits related to cosmetic appearance of the hair, as well as growth thereof. The further hair care active component may provide an immediate and short lived (i.e. acute) benefit, and/or a long term and long lasting (i.e. chronic) benefit.

Also envisaged is the use of the topical composition described herein as supportive treatment for subjects that underwent hair transplant surgery. In such embodiments, the topical composition may be applied after the hair transplant is conducted, before the hair transplant is conducted, or both. The topical composition may be applied to the transplant area of the scalp (donor and/or recipient area), or the complete scalp of the subject.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects and embodiments of the invention are further supported by the following non-limiting examples.

The following specific experimental examples are provided in support of the claimed invention but are not to be seen as limiting the scope of the invention.

### EXAMPLES

### Example 1. Manufacturing Processes

### Aqueous Gel Formulations

### Batches AG23, AG24, AG25, AG26

i. Water (first addition) was weighed into a Duran (vessel 1).
ii. The sample from Step (i) was placed on a hot plate stirrer at 800 rpm forming a vortex.
iii. The polymer (Carbopol®) was weighed via a weighing boat and dispersed in the contents of Step (ii) maintaining the vortex. The sample was left on stir for at least an hour to allow polymer dispersion.
iv. The weight of polymer dispersion in Step (iii) was recorded.
v. The sample from Step (iv) was autoclaved at standard conditions (121° C.±2° C., 2×10⁵ Pa, 15 mins).
vi. Once the autoclaved sample was cooled, the sample from Step (v) was re-weighed and additional water was added to compensate volatile loss from evaporation.
vii. The solvents (PEG 400, Transcutol® P (diethylene glycol monoethyl ether) were weighed into a separate vessel.
viii. Estetrol monohydrate was weighed into the contents of Step (vii) and stirred at 500 rpm for 2.5 hours (hotplate magnetic stirrer at laboratory room temperature) to dissolve the drug.
ix. In the biosafety laminar flow hood, the contents of Step (viii) were filtered through multiple Spartan (regenerated cellulose) 0.2 μm sterile filters into multiple 20 mL autoclaved vials, this was to avoid the contamination of the bulk should a filter break.
x. The contents of Step (ix) were combined and weighed into a pre-autoclaved Duran vessel.
xi. The contents of Step (x) were poured into the contents of Step (vi) and mixed with an overhead stirrer and spatula; the mixing speed and time were recorded in the laboratory specific notebook.
xii. Water (ca. 4% of the total amount) was used to rinse the vessel from Step (x) and poured into vessel 1.
xiii. The formulation was left to rest for at least overnight xiv. The pH of the sample was adjusted between the target pH, 6-6.5 with sodium hydroxide solution and taken to weight with water.
xv. It is to be noted that manufacturing Steps (ix) to (xiv) were performed under aseptic conditions in a laminar flow hood.

### Batches AG30, AG22 (Carbopol® 980)

i. Water (first addition) was weighed into a 150 mL Duran (vessel 1).
ii. The sample from Step (i) was placed on a hot plate stirrer at 800 rpm forming a vortex.
iii. The polymer (Carbopol® 980) was weighed via weighing boat and dispersed in the contents of Step (ii) maintaining the vortex. The sample was left on stir for at least an hour to allow polymer dispersion.
iv. The weight of polymer dispersion in Step (iii) was recorded.
v. PEG 400 and glycerol was weighed and added to the contents of Step (iv).
vi. The sample from Step (v) was autoclaved at standard conditions (121° C.±2° C., 2×10⁵ Pa, 15 mins).
vii. Once the autoclaved sample cooled, the sample from Step (v) was re-weighed and additional water was added to compensate volatile loss from evaporation.
viii. The solvents (propylene glycol (PG), Transcutol® P) were weighed into a 100 mL Duran.
ix. Estetrol monohydrate was weighed into the contents of Step (viii) and stirred at 500 rpm (hotplate magnetic stirrer at laboratory room temperature).
x. In the biosafety laminar flow hood, the contents of Step (ix) was filtered through multiple Spartan 0.2 μm sterile filters into multiple 20 mL autoclaved vials to avoid the contamination of the bulk should a filter break.
xi. The contents of Step (x) were combined and weighed into a pre-autoclaved 100 mL Duran.
xii. Sodium hydroxide 18% (previously filtered through a 0.2 μm sterile Spartan filter) was poured into the contents of Step (xi) and mixed.
xiii. The contents of Step (xii) was swiftly poured into the contents of Step (vii) and mixed with a spatula.
xiv. Water (ca. 4% of the total amount) was used to rinse the vessel from Step (x) and poured into vessel
xv. The thickened sample was left to rest for at least overnight.
xvi. The pH of the sample was measured, and the remaining sterile water was added, alongside sodium hydroxide solution (also previously sterilized through a 0.2 μm Spartan filter) to adjust to pH 7-7.5 where required.

It is to be noted that manufacturing Steps (x) to (xvi) were performed under aseptic conditions in a laminar flow hood.

### Batch AG27 (Poloxamer)

i. Water (first addition) was weighed into a 150 mL Duran and cooled in a fridge maintained at 2-8° C. for 20 minutes.
ii. The sample from Step (i) was placed on a hot plate stirrer and poloxamers (P407 followed by P188) were weighed and added over a vortex at 800 rpm.
iii. The sample from Step (ii) was stirred with the aid of an overhead stirrer for 6 hours at 200 rpm until the poloxamer dissolved in the water bath maintained at 2-8° C.
iv. In a separate vessel, PEG 400, benzyl alcohol (BA) and PG were weighed together.
v. Estetrol monohydrate was weighed and added to the contents of Step (iv) and left to stir overnight in a hot plate stirrer at 500 rpm.
vi. Once the drug dissolved, the required amount of solution of Step (v) was weighed onto the first vessel containing the poloxamer solution. The sample was left on a magnetic stirrer overnight at 300 rpm.
vii. The sample from Step (vi) was pH adjusted to 7-7.5.

### Batches AG28 (Hydroxyethyl Cellulose (HEC)), AG29 (Carboxymethyl Cellulose (CMC))

i. The solvent system for the formulation was prepared by weighing out all solvents, except water/buffer for the active systems only.
ii. The drug was added to the solvent systems for the active formulations and put on stir at 500 rpm overnight.
iii. Following the dissolution of the drug, whilst the Duran is on stir, the water/pH 7.0 buffer solution was added to the solvent system.
iv. A suitable amount of premix was weighed into a separate 150 mL Duran.
v. The polymer was dispersed whilst forming a vortex stirring at 800 rpm and the formulation was left to stir overnight at 500 rpm.
vi. For AG28, the pH was adjusted to pH 7-7.5, pH adjustment was not required for AG29 as it contains buffer phosphate-phosphate pH 7.0.

### Batch AG15 (Poloxamer)

i. Water (first addition) was weighed into a 150 mL Duran and cooled in a fridge maintained at 2-8° C. for 20 minutes.
ii. The sample from Step (i) was placed on a hot plate stirrer and poloxamers (P407 followed by P188) were weighed and added over a vortex at 800 rpm.
iii. The sample from Step (ii) was stirred with the aid of an overhead stirrer for 6 hours at 200 rpm until the poloxamer dissolved in the water bath maintained at 2-8° C.
iv. In a separate vessel, PEG400, BA and PG were weighed together.
v. Estetrol was weighed and added to the contents of Step (iv) and left to stir overnight in a hot plate stirrer at 500 rpm. This step was omitted for the manufacture of placebo.
vi. Once the drug dissolved, the required amount of solution of Step (v) was weighed onto the first vessel containing the poloxamer solution. The sample was left on a magnetic stirrer overnight at 300 rpm.
vii. The sample from Step (vi) was pH adjusted to 7-7.5.

### Batch AG17

i. Water (first addition) was weighed into a 150 mL Duran (vessel 1).
ii. The sample from Step (i) was placed on a hot plate stirrer at 800 rpm forming a vortex.
iii. The polymer (Carbopol®) was weighed via weighing boat and dispersed in the contents of Step (ii) maintaining the vortex. The sample was left on stir for at least an hour to allow polymer dispersion.
iv. The weight of polymer dispersion in Step (iii) was recorded.
v. The sample from Step (iv) was autoclaved at standard conditions (121° C.±2° C., 2×10⁵ Pa, 15 mins).
vi. Once the autoclaved sample cooled, the sample from Step (v) was re-weighed and additional water was added to compensate volatile loss from evaporation.
vii. Benzyl alcohol, PEG 400 and PG were weighed into a separate 100 mL Duran.
viii. Estetrol monohydrate was weighed into the contents of Step (vii) and stirred at 500 rpm overnight (hotplate magnetic stirrer at laboratory room temperature) to dissolve the drug. This step was omitted for the placebo formulation
ix. Sodium hydroxide 18% (1.38 g) was poured into the contents of Step (vi) and mixed.
x. The contents of Step (ix) were swiftly poured into the contents of Step (vi) and mixed with a spatula.
xi. Water (4.5 g) was used to rinse the vessel from Step (ix) and poured into vessel 1.
xii. The sample which had thickened was left to rest for at least overnight.
xiii. The pH of the sample was measured, and the remaining water was added gradually whilst continuing to measure the pH.

### Batches AG18 (HEC), AG19 (CMC)

i. The solvent systems for placebo and active formulations were prepared by weighing out all solvents, except water/buffer for the active systems only.
ii. The drug was added to the solvent systems for the active formulations and put on stir at 500 rpm overnight.
iii. Following the dissolution of the drug, whilst the Duran was on stir, the water/pH 7.0 buffer solution was added to the solvent system.
iv. A suitable amount of premix was weighed into a separate 150 mL Duran.

The polymer was dispersed whilst forming a vortex stirring at 800 rpm and the formulation was left to stir overnight at 500 rpm.
v. For AG18, the pH was adjusted to pH 7-7.5, pH adjustment was not required for AG19 as it contained buffer phosphate-phosphate pH 7.0.

### Batches AG20, AG23

i. Steps (i)-(vi) from AG17 were followed.
ii. The solvents (PEG 400, Transcutol® P) were weighed into a separate vessel (100 mL).
iii. Estetrol monohydrate was weighed into the contents of Step (ii) and stirred at 500 rpm for 2.5 hours (hotplate magnetic stirrer at laboratory room temperature) to dissolve the drug. This step was omitted for the placebo formulation.
iv. In the biosafety laminar flow hood, the contents of Step (iii) were filtered through multiple Spartan (regenerated cellulose) 0.2 μm sterile filters into multiple 20 mL autoclaved vials, this was to avoid the contamination of the bulk should a filter break. It should be noted that initially the samples were going to be filtered through the Nalgene bottle system filter (PES), with the aid of a vacuum pump but the filters broke, hence Spartan syringe filters were used.
v. The contents of Step (iv) were combined and weighed into a pre-autoclaved 100 mL Duran.
vi. The contents of Step (v) were poured into the contents of Step (i) and mixed with a spatula.
vii. Water (4.5 g) was used to rinse the vessel from Step (v) and poured into vessel 1.
viii. As the formulation did not thicken, it was left to rest for at least overnight.
ix. The pH of the sample was adjusted between 6-6.5 with sodium hydroxide solution and taken to weight with water. It is to be noted that initially a process with sodium hydroxide 18% was performed, however, Carbopol® clumped or failed to hydrate. Furthermore, the original target pH was 7-7.5, however, precipitation of the polymer was observed above pH 6.5, samples were repeated and left between pH 6-6.5 to avoid this issue.

It is to be noted that manufacturing Steps (iv) to (ix) were performed under aseptic conditions in a laminar flow hood.

### Batches AG21, AG22

i. Steps (i) to (vi) from AG17 were followed.
ii. The solvents (PEG 400, PG, Trans P) were weighed into a 100 ml Duran.
iii. Estetrol monohydrate was weighed into the contents of Step (i) and stirred at 500 rpm (hotplate magnetic stirrer at laboratory room temperature). The drug dissolved within 2.5 hours of stirring for AG22 and overnight for AG21. This step was omitted for the placebo formulations.
iv. In the biosafety laminar flow hood, the contents of Step (viii) were filtered through multiple Spartan 0.2 μm sterile filters into multiple 20 mL autoclaved vials to avoid the contamination of the bulk should a filter break.
v. The contents of Step (ix) were combined and weighed into a pre-autoclaved 100 mL Duran.
vi. Sodium hydroxide 18% (1.38 g) (previously filtered through a 0.2 μm sterile Spartan filter) was poured into the contents of Step (x) and mixed.
vii. The contents of Step (xi) were swiftly poured into the contents of Step (vi) and mixed with a spatula.
viii. Water (4.5 g) was used to rinse the vessel from Step (x) and poured into vessel 1.
ix. The sample thickened and was left to rest for at least overnight.
x. The pH of the sample was measured, and the remaining sterile water was added, along sodium hydroxide solution (also previously sterilized through a 0.2 μm Spartan filter) to adjust to pH 7-7.5 where required.

It is to be noted that manufacturing Steps (ix) to (xv) were performed under aseptic conditions in a laminar flow hood.

TABLE 1
Compositions (% w/w) of estetrol monohydrate (E4) aqueous gel formulations.

| Formulation | AG15 | AG17 | AG18 | AG19 | AG20 | AG21 | AG22 | AG23 |
|---|---|---|---|---|---|---|---|---|
| Estetrol | 0.24 | 0.5 | 0.5 | 0.5 | 0.44 | 0.37 | 0.06 | 0.5 |
| monohydrate (E4) | | | | | | | | |
| Buffer phosphate- | — | — | — | 48.00 | — | — | — | — |
| phosphate pH 7.0 | | | | | | | | |
| PEG 400 | 14.18 | 18.00 | 18.00 | 18.00 | 45.00 | 35.00 | 10.00 | 50.00 |
| Propylene glycol | 14.18 | 20.00 | 20.00 | 20.00 | — | 5.00 | — | — |
| Transcutol ® P | — | — | — | — | — | — | 5.00 | 5.00 |
| Glycerol | — | 10.00 | 10.00 | 10.00 | — | 5.00 | — | — |
| Benzyl alcohol | 2.00 | 2.00 | 2.00 | 2.00 | — | — | — | — |
| Water (1st | 45.00 | 40.00 | 40.00 | — | 45.00 | 45.00 | 75.00 | 35.00 |
| Addition) | | | | | | | | |
| Carbopol ® 980 | — | 0.50 | — | — | 0.50 | 0.50 | 0.50 | 0.50 |
| HEC 250 HHX | — | — | 1.50 | — | — | — | — | — |
| CMC | — | — | — | 1.50 | — | — | — | — |
| Poloxamer 188 | 3.75 | — | — | — | — | — | — | — |
| Poloxamer 407 | 15.00 | — | — | — | — | — | — | — |
| Sodium hydroxide | — | Adjust to | | — | Adjust to | | | Adjust to pH 6-6.5 |
| | | pH 7-7.5 | | | pH 7-7.5-7.5 | | | |
| Citric acid 0.1M* | Adjusted | — | — | — | — | — | — | — |
| | to pH | | | | | | | |
| | 7-7.5 | | | | | | | |
| Water (2^{nd} | q.s. 100% | | | — | q.s. 100% | | | |
| addition) | | | | | | | | |
| TOTAL | | | | | | | | |

| Formulation | AG24 | AG25 | AG26 | AG27 | AG28 | AG29 | AG30 |
|---|---|---|---|---|---|---|---|
| Estetrol | 0.50 | 0.22 | 0.06 | 0.06 | 0.06 | 0.06 | 0.06 |
| monohydrate (E4) | | | | | | | |
| Buffer phosphate- | — | — | — | — | — | 48.44 | — |
| phosphate pH 7.0 | | | | | | | |
| PEG 400 | 43.95 | 33.05 | 20 | 14.18 | 18.00 | 18.00 | 35.00 |
| Propylene glycol | — | — | — | 14.18 | 20.00 | 20.00 | 5.00 |
| Transcutol ® P | 1.00 | 1.00 | 5.00 | — | — | — | — |
| Glycerol | — | — | — | — | 10.00 | 10.00 | 5.00 |
| Benzyl alcohol | — | — | — | 2.00 | 2.00 | 2.00 | — |
| Water (1st | 43.24 | 55.45 | 63.27 | 45.00 | 40.00 | — | 45.00 |
| Addition) | | | | | | | |
| Carbopol ® 980 | 0.50 | 0.50 | 0.50 | — | — | — | 0.50 |
| HEC 250 HHX | — | — | — | — | 1.50 | — | — |
| CMC | — | — | — | — | — | 1.50 | — |
| Poloxamer 188 | — | — | — | 3.75 | — | — | — |
| Poloxamer 407 | | | | 15.00 | — | — | — |
| Sodium hydroxide | Adjust to pH 6-6.5 | | | Adjust to | | — | Adjust |
| | | | | pH 7-7.5 | | | pH 7-7.5 |
| Citric acid 0.1M* | — | — | — | — | — | — | — |
| Water (2^{nd} | q.s. 100% | | | | | — | q.s. to |
| addition) | | | | | | | 100% |
| TOTAL | | | | | | | |

### Creams

### Batches CR01, CR05, CR12

i. Placebo and active solvent systems were prepared by weighing out all solvents, except water/buffer for the active systems only.
ii. The drug was added to the solvent systems for the active creams and put on stir at 500 rpm overnight.
iii. After the drug had completely dissolved, whilst the container (Duran) was on stir, the water/pH 7.0 buffer solution was added to the solvent system.
iv. The oil phases were then prepared. For CR01, the oil phase was placed in the water bath at 75° C. until it was melted.
v. For CR05 and CR12, the oil phases were put in the oven for 2 hours at 160° C. (to mimic the process of sterilization). Once the oil phases had cooled to room temperature, and re-solidified, they were placed in the water bath (75° C.) until the oil phases melted again.
vi. Before processing the active and placebo creams, the required amount of the solvent system pre-mix was weighed out into a separate Duran.
vii. The Durans with the pre-mix was placed in the water bath along with the homogenizer head for 5 minutes.
viii. For CR01 and CR12, the creams were processed via an Ultra-turrax equipped with a 25G dispersing head at 10,000 rpm for 2 minutes.
ix. For CR05, during homogenization, the cream was homogenized at 5000 rpm, for 5 minutes instead, to avoid overflowing.
x. After processing, the creams were hand stirred with a metal spatula until the cream reached room temperature.
xi. They were left to cure overnight.
xii. Formulations were pH adjusted to pH 7-7.5.

### Batches CR10, CR13, CR14, CR15, CR16

i. Placebo and active solvent systems were prepared by weighing out all solvents, except water/buffer for the active systems only.
ii. The drug was added to the solvent systems for the active creams and put on stir at 500 rpm overnight.
iii. After the drug had completely dissolved, whilst the Duran was on stir, the water/pH 7.0 buffer solution was added to the solvent system.

The oil phases of CR13, CR14 and CR15 were put in the oven for 2 hours at 160° C. (sterilized) and left to re-solidify at room temperature.
v. The aqueous phases were sterilized by filtration using sterile PES syringe filters 0.2 μm.
vi. The required amount of aqueous phase was weighed into a 250 mL Duran which was pre-sterilized by autoclaving.
vii. The oil phases were placed in a water bath at 75° C. until molten.
viii. Aqueous phases placed in the water bath along with the homogenizer head to equilibrate for 5 minutes.
ix. For each cream, oil phase was added to the respective aqueous phase and homogenized using an Ultra-turrax equipped with a 25G dispersing head at 10,000 rpm for 2 minutes.
x. After processing, the creams were hand stirred with a metal spatula until the cream reached room temperature.
xi. They were left to cure overnight.
xii. Formulations were pH adjusted to pH 7-7.5

It is to be noted that manufacturing Step (v) to (xii) were performed under aseptic conditions in a laminar flow hood and the sterile vessels were only opened in sterile laminar flow hood.

TABLE 2
Compositions (% w/w) of estetrol monohydrate (E4) cream formulations.

| Formulation | CR01 | CR05 | CR07 | CR08 | CR09 | CR10 | CR11 | CR12 | CR13 | CR14 | CR15 | CR16 |
|---|---|---|---|---|---|---|---|---|---|---|---|---|
| Estetrol | 0.24 | 0.24 | 0.28 | 0.21 | 0.35 | 0.50 | 0.06 | 0.28 | 0.21 | 0.35 | 0.06 | 0.50 |
| monohydrate (E4) | | | | | | | | | | | | |
| Water | 49.76 | 49.76 | 39.72 | 44.79 | 39.65 | — | 64.94 | 29.72 | 34.79 | 29.65 | 54.94 | 34.50 |
| Buffer phosphate- | — | — | — | — | — | 39.50 | — | — | — | — | — | — |
| phosphate pH 7.0 | | | | | | | | | | | | |
| PEG 400 | 14.00 | 14.00 | — | 25.00 | 30.00 | 40.00 | 10.00 | — | 25.00 | 30.00 | 10.00 | 40.00 |
| Propylene glycol | 14.00 | 14.00 | 28.00 | — | — | — | — | 28.00 | — | — | — | — |
| Transcutol ® P | — | — | — | — | 5.00 | — | 5.00 | — | — | 5.00 | 5.00 | — |
| Glycerol | — | — | 10.00 | 10.00 | 5.00 | — | — | 10.00 | 10.00 | 5.00 | — | — |
| Benzyl alcohol | 2.00 | 2.00 | 2.00 | — | — | — | — | 2.00 | — | — | — | — |
| Polawax ® NF | — | — | — | — | — | 7.00 | — | — | — | — | 7.00 | — |
| Glycerol | — | 1.31 | — | — | — | — | — | 1.31 | 1.31 | 1.31 | 1.31 | — |
| Monostearate | | | | | | | | | | | | |
| Brij S2 | 1.02 | — | 1.02 | 1.02 | 1.02 | 0.57 | 1.02 | — | — | — | — | 0.57 |
| Brij S721 | 2.98 | — | 2.98 | 2.98 | 2.98 | 3.43 | 2.98 | — | — | — | — | 3.43 |
| Cetomacrogol ® | — | 2.69 | — | — | — | — | — | 2.69 | 2.69 | 2.69 | 2.69 | — |
| 1000 | | | | | | | | | | | | |
| Cetyl alcohol | 4.00 | — | 4.00 | 4.00 | 4.00 | — | 4.00 | — | — | — | — | — |
| Stearic acid | 4.00 | — | 4.00 | 4.00 | 4.00 | — | 4.00 | — | — | — | — | — |
| Cetostearyl | — | 5.00 | — | — | — | — | — | 5.00 | 5.00 | 5.00 | 5.00 | — |
| alcohol | | | | | | | | | | | | |
| Castor oil | — | — | — | — | — | 9.00 | — | — | — | — | — | 9.00 |
| Mineral oil | 8.00 | 11.00 | 8.00 | 8.00 | 8.00 | — | 8.00 | 11.00 | 11.00 | 11.00 | 11.00 | — |
| Water (2nd | q.s. to 100 | | | | | | | | | | | |
| addition) | | | | | | | | | | | | |
| Sodium hydroxide | Adjust to pH 7.0-7.5 | | | | | | | | | | | |
| solution | | | | | | | | | | | | |
| Total | 100.00 | 100.00 | 100.00 | 100.00 | 100.00 | 100.00 | 100.00 | 100.00 | 100.00 | 100.00 | 100.00 | 100.00 |

### Example 2. In Vitro Release Experiment 1

Following method development and a small scale preliminary in vitro release testing (IVRT) experiment, a full scale IVRT experiment was performed using 10 test formulations described in Example 1. The experimental conditions utilized are shown in Table 3.

TABLE 3
Experimental conditions for the full-scale IVRT.

| Parameter | Experimental conditions |
|---|---|
| No. of formulations | 10 |
| Replicates | n = 6 active; n = 1 blank |
| Membrane | isopore |
| Receptor solution | 40:30:30 v/v/v Ethanol:PEG400:Water |
| RS Sampling volume | 1.0 mL |
| Dose Amount | 0.3 g |
| Stir rate | 50 rpm |
| Sampling Timepoints | t = 0, 30, 60, 120, 180, 240, 300, 360, |
| | 420, 480 minutes |
| Membrane temperature | 32° ± 1° C. |

The full scale in vitro release experiment was performed using the experimental conditions developed during the method development and feasibility experiments. The results of all tested gel and cream formulations are shown in FIG. 1, with tabulated results shown in Table 4.

### Aqueous Gel Formulations

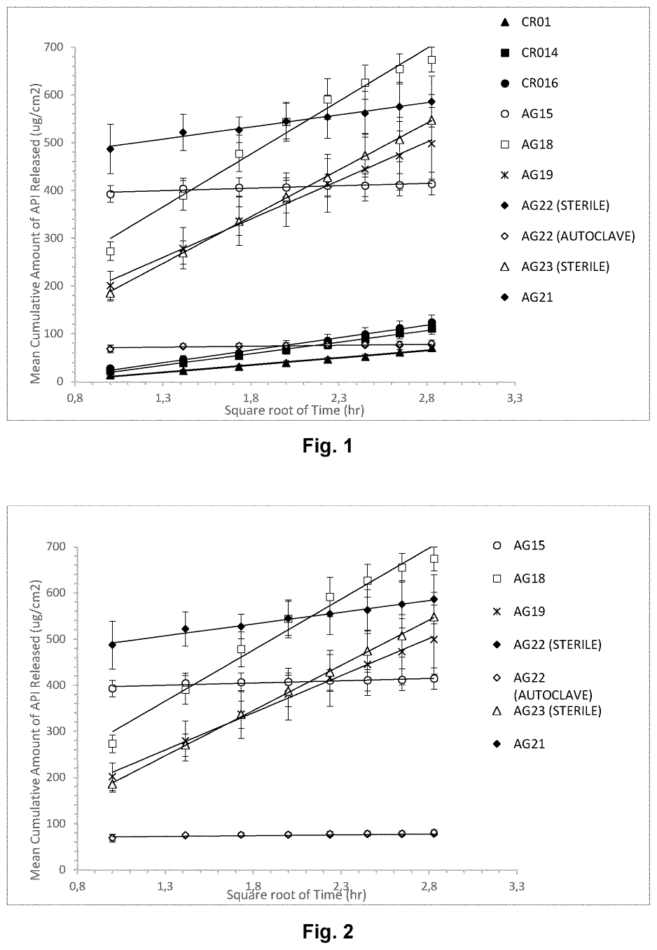

The release rates of the aqueous gel formulations are presented in FIG. 2 and table 4. The AG18, AG19, and AG23 formulations resulted in the highest release rates (161-221 μg/cm²/√hr), followed by AG21 (51 μg/cm²/√hr), with AG22 (sterile), AG22 (autoclave), and AG15 resulting in the lowest release rates (3-10 μg/cm²/√hr). As a general trend, the release rates observed correlate with the concentration of API, with the formulations with the highest release rates (AG18, AG19, and AG23) also containing the largest concentration of estetrol monohydrate (0.50% w/w).

The aqueous gels with the lowest release rates (AG21, AG22 (sterile), AG22 (autoclave), and AG15) were found to immediately release a large amount of the dosed API (ca. 50% or greater release at t=0.5 hr) into receptor solution. As a result of the large release of API, linear steady state drug release was not achieved (r2<0.9) for many of the formulations. Therefore, conclusions and statistical comparisons were not performed using the aqueous gel formulations.

### Cream Formulations

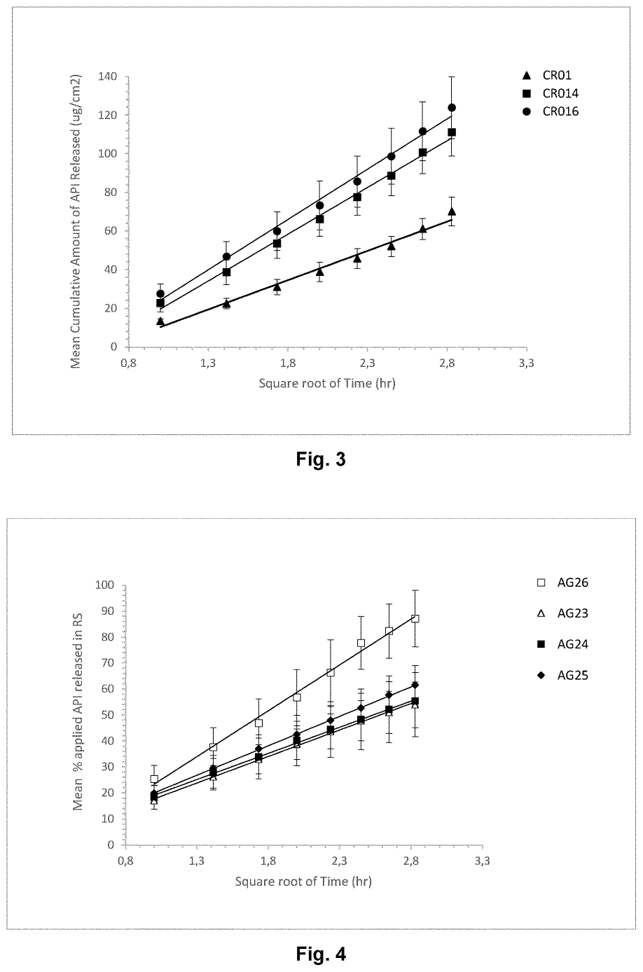

The release rates of the cream formulations are presented in FIG. 3 and complemented by table 4. CR16 (0.50% w/w) resulted in the highest release rate, followed by CR14 (0.35% w/w), and finally CR01 (0.24% w/w), with average release rates ranging from 32-53 μg/cm2/√hr. Following the same trend as the aqueous gels, the release rates correlated with the concentration of API present in the formulation, with the highest release rate observed in the formulation with the highest drug loading (CR16, containing 0.50% API and resulting in a release rate of 52.20 g/cm²/√hr). One-way statistical analysis of the mean drug release (μg/cm²/√hr) of the cream formulations was performed using Tukey-Kramer (n=6). There were no statistical differences between the release rates of CR14 and CR16 (p>0.05), whereas the release rate of CR01 was observed to have a significantly lower release rate (p<0.05).

TABLE 4
Mean estetrol monohydrate release rate (μg/cm²/√hr)
between the 1 and 8 hr experimental timepoints from
10 formulations across an Isopore ® membrane
in receptor solution 40:30:30 v/v/v ethanol:PEG400:water.

| API Release Rate (slope; μg/cm²/√hr) | | | | Linearity (r2) | | |
|---|---|---|---|---|---|---|
| Formulation | n | Mean | Std Dev | n | Mean | Std Dev |
| AG22 | 6 | 3.85 | 2.33 | 6 | 0.74 | 0.09 |
| (Sterile) | | | | | | |
| AG22 | 6 | 5.54 | 4.36 | 6 | 0.77 | 0.13 |
| (autoclave) | | | | | | |
| AG15 | 6 | 9.97 | 4.75 | 6 | 0.75 | 0.25 |
| AG21 | 6 | 50.75 | 27.27 | 6 | 0.90 | 0.06 |
| AG19 | 6 | 161.01 | 24.52 | 6 | 1.00 | 0.00 |
| AG23 | 6 | 195.69 | 20.22 | 6 | 1.00 | 0.00 |
| (sterile) | | | | | | |
| AG18 | 6 | 220.93 | 6.96 | 6 | 0.98 | 0.02 |
| CR01 | 6 | 30.41 | 3.41 | 6 | 0.98 | 0.02 |
| CR14 | 6 | 48.51 | 4.18 | 6 | 0.99 | 0.00 |
| CR16 | 6 | 52.20 | 6.34 | 6 | 0.99 | 0.01 |

### Example 3. In Vitro Release Experiment 2

Following the results of the first in vitro release experiment (Example 2), a second IVRT experiment was performed using the 4 formulations and experimental parameters outlined in Table 5.

TABLE 5
IVRT study parameters and formulations employed
in in vitro drug release experiment 2.

| IVRT Parameters | Experimental conditions |
|---|---|
| No. of formulations | Four: |
| | AG23 (0.50% ACT) |
| | AG24 (0.50% ACT) |
| | AG25 (0.22% ACT) |
| | AG26 (0.06% ACT) |
| Replicates | n = 6 per active |
| Membrane | Isopore ® |
| Receptor solution | 40:30:30 v/v/v Ethanol:PEG400:water |
| RS Sampling volume | 1.0 mL |
| Dose amount | 0.3 g |
| Stir rate | 50 rpm |
| Sampling Timepoints | 0, 30, 60, 120, 180, 240, 300, 360, 420 and |
| | 480 min |
| Membrane temperature | 32 ± 1° C. |

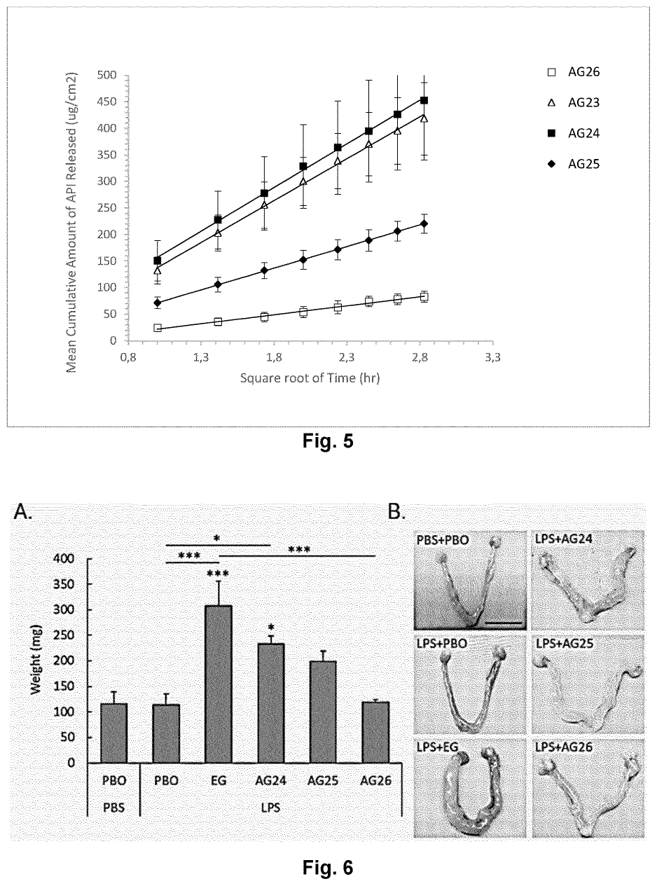

The second full scale in vitro release experiment was performed using the experimental parameters detailed in Table 5 and four aqueous gel formulations. The results of all tested formulations are shown in FIG. 4 and FIG. 5, with tabulated results shown in Table 6 and Table 7.

The purpose of this part of the study was to determine the effect of thermodynamic activity on the rate of release of the estetrol monohydrate from the formulations by comparing a non-optimised formulation (AG23 0.5% (w/w) API) to optimised formulations at a variety of strengths (AG24 0.5% (w/w), AG25 0.22% (w/w) and AG26 0.06% (w/w) API).

When considering these 4 formulations in terms of percentage release of applied dose of API (Table 7), 87.95±10.88% of API was released from formulation AG26 (0.06% (w/w) API) after 8 h, which was significantly higher (p, 0.05) than from the other 3 formulations which were not statistically different to each other (p>0.05) (61.51±4.91% AG25 (0.22% (w/w) API), 60.67±4.86% AG24 (0.5% (w/w) API) and 56.88±5.90% AG23 (0.5% (w/w) API)).

Considering the percentage of applied dose release rates over time (slope) there was a significant difference (p<0.05) between formulation AG26 and the other three formulations (AG23, AG24 and AG25). No statistically significant differences were found between AG23, AG24 and AG25. Statistical analysis was performed by Tukey-Kramer test (n=5-6).

During the current study, formulation AG23 displayed a release rate of ca. 167±13 μg/cm²/hr compared to 195±20 μg/cm²/hr in the first IVRT full scale experiment. Formulation AG24 had the highest release rate (ca. 182±8 μg/cm²/hr) in the current study, followed by AG23, AG25 (ca. 81±5 μg/cm²/hr) and finally AG26 (ca. 34±3 μg/cm²/hr). The release rate of each formulation is significantly different (p<0.05) to the next, which in the case of AG23 and AG24, which both contain 0.5% (w/w) estetrol monohydrate, could be due to the issues encountered dosing AG23 or batch to batch variability compared to the batch of AG23 used in the previous experiment (which had a release rate much closer to the one seen here for AG24), or simply a formulation difference between AG23 and AG24. For the other two formulations the difference in release rate is as expected due to the lower API content in these formulations (AG23 and AG24 0.5% w/w, AG25 0.22% (w/w) and AG26 0.06% w/w).

To assess the differences between the aqueous gel formulations as reported above, a one-way statistical analysis was performed using Tukey-Kramer (n=5-6).

TABLE 6
Mean estetrol monohydrate release rate (μg/cm²/√hr)
between the 1 and 8 hr experimental timepoints from
aqueous gel formulations across an isopore membrane
in receptor solution 40:30:30 v/v/v ethanol:PEG400:water.

| API Release Rate (slope; ug/cm²/√hr) | | | | Linearity (r2) | | |
|---|---|---|---|---|---|---|
| Formulation | n | Mean | Std Dev | n | Mean | Std Dev |
| AG24 (0.50%) | 5* | 182.02 | 7.69 | 5 | 1.00 | 0.01 |
| AG23 (0.50%) | 5* | 167.19 | 13.43 | 5 | 1.00 | 0.00 |
| AG25 (0.22%) | 6 | 81.32 | 5.13 | 6 | 1.00 | 0.00 |
| AG26 (0.06%) | 6 | 33.61 | 3.05 | 6 | 0.98 | 0.02 |
| AG23** | 6 | 195.69 | 20.22 | 6 | 1.00 | 0.00 |

*Single replicate removed as identified as an outlier by Dixon's Q test
**Data from full scale IVRT experiment 1

TABLE 7
Cumulative estetrol monohydrate (μg/cm²) and percentage
of applied dose released at the 8 hr experimental timepoint
from aqueous gel formulations across an isopore membrane
in receptor solution 40:30:30 v/v/v ethanol:PEG400:water.

| | | | | | Percentage of applied | |
|---|---|---|---|---|---|---|
| Cumulative Amount of API Released (ug/cm²) | | | | | API released | |
| Formulation | n | Mean | Std Dev | n | Mean | Std Dev |
| AG24 | 5* | 495.91 | 39.72 | 5 | 60.67% | 4.86% |
| AG23 | 5* | 440.72 | 45.69 | 5 | 56.88% | 5.90% |
| AG25 | 6 | 220.43 | 17.58 | 6 | 61.51% | 4.91% |
| AG26 | 6 | 82.95 | 10.36 | 6 | 87.16% | 10.88% |

*Single replicate removed as identified as an outlier by Dixon's Q test

### Example 4. In Vivo LPS Treatment Protocol (Delayed Wound Healing Model)

In order to assess systemic effect of topically applying estrogen comprising compositions, the in vivo LPS-induced delayed wound healing model was used as previously described (Crompton R, Williams H, Ansell D, Campbell L, Holden K, Cruickshank S, Hardman MJ. Oestrogen promotes healing in a bacterial LPS model of delayed cutaneous wound repair. Lab Invest. 2016 April; 96 (4): 439-49).

Female wild-type (C57BL/6J) 8 weeks old mice were split into six groups (6 animals per experimental group):
Double placebo control (no LPS, placebo treatment),
LPS control (LPS, placebo treatment),
LPS with topical EstroGel® (estradiol gel, contains 0.06% 17ß-estradiol as hemihydrate in a hydro-alcoholic gel),
LPS with topical AG24 (E4 at 0.5%),
LPS with topical AG25 (E4 at 0.22%),
LPS with topical AG26 (E4 at 0.06%).

The day prior to wounding (day-1), all the animals were weighed and anesthetized using oxygen and isoflurane (flow rate 1.25-2 L with 2-2.5% isoflurane depending on clinical signs). Animals were shaved and dorsal areas prepped. Wound positions (2 wounds) were marked on the dorsum of each animal. Animals received a first subcutaneous injection of 2 μg K. pneumoniae-derived LPS diluted in DPBS (1 μg per wound; Sigma Aldrich, UK: L4268) at the wound site, except the double placebo group that received DPBS only. A thin layer of EstroGel®, AG24, AG25, AG26 or placebo were then applied to the dorsum of each animal, measured as 60 μl total (30 μl/wound). Animals recovered in a warming cabinet and were then single housed in fresh cages containing alpha pad, RO water, food, mash and a house. On day 0 (24 hours after first anesthesia), mice were re-anesthetized and dorsal skin cleaned with chlorhexidine wipes. Two 6 mm dorsal excisions were created. 2 μg LPS (1 μg per wound) was subcutaneously injected at the wound site as above, except for the double placebo mice that were injected with DPBS. EstroGel®, AG24, AG25, AG26 or AG23as Placebo was then applied in a thin layer over the top of the wound (60 μl total; 30 μl/wound), being careful not to damage the “LPS bleb”. Buprenorphine (0.1 mg/Kg), an analgesic, was administered post-operatively via subcutaneous injection in the scruff and each animal was imaged. Mice were recovered in a warming cabinet and returned to single housing. Observations were performed post-operatively.

On days 1, 2, 3 and 4, for treated groups, topical EstroGel®, AG24, AG25, AG26 or placebo were re-applied in a thin layer over the top of the wounds (60 μl total; 30 μl/wound) during observations.

On day 5, mice were humanely culled via rising concentration of CO₂ and cervical dislocation. The uteri were removed and weighed.

It is apparent from FIG. 6 that topical application of EstroGel® promoted uterine weight gain and hypertrophic changes. Topical application of E4 affects uterine weight in a dose-dependent manner, with AG26 showing no effect and AG24 inducing uterine weight gain. The data confirm that topical application of low concentration E4 formulations (AG26) to open wounds for 6 days, in contrast to EstroGel® (E2) and high-level E4 formulations (AG24, AG25), did not result in systemic side effects, expressed in the increase in uterus weight.

Based on the data depicted in FIG. 6 we conclude that the topically applied AG26 formulation does not has systemic effects such as uterine weight increase. This is indicative of the preferred dosage range if systemic effects are to be avoided completely. In some cases however, a higher dosage may be envisaged based on a favorable risk/benefit for the subject at hand.

### Example 5. In Vivo LPS Treatment Protocol (Delayed Wound Healing Model): Comparison of Different Formulations and Variation of Treatment Duration

The in vivo LPS treatment protocol (delayed wound healing model) as presented in example 4 has been repeated with the following groups of animals:
Double placebo group (untreated control),
LPS control (LPS, placebo treatment),
LPS with topical EstroGel® (estradiol gel, contains 0.06% 17β-estradiol as hemihydrate in a hydro-alcoholic gel), repeated administration for 4 days,
LPS with topical AG26 (E4 at 0.06%), single administration,
LPS with topical AG26 (E4 at 0.06%), repeated administration for 4 days,
LPS with topical AG28 (E4 at 0.06%), single administration,
LPS with topical AG28 (E4 at 0.06%), repeated administration for 4 days.

On day 0 only (single administration), or on days −1, 0, 1 and 2 (repeated administration), topical EstroGel®, AG26, AG28 or corresponding placebo was applied in a thin layer over the top of the wounds (60 μl total; 30 μl/wound) during observations.

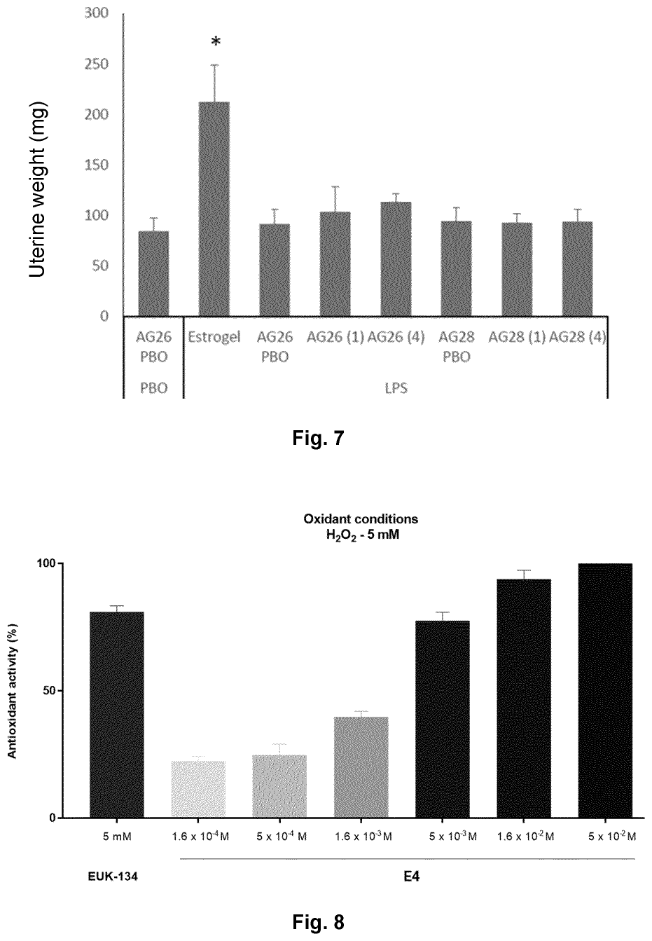

On day 3, mice were humanely culled via rising concentration of CO₂ and cervical dislocation. The uteri were removed and weighed as described under example 4. In contrast to EstroGel®, topical application of AG26 and AG28 did not affect uterine weight (FIG. 7).

### Example 6: Evidence of the Antioxidant Property of Estetrol

The antioxidant activity of E4 was evaluated using the Skin-Biosense® sensor, a technology developed by Qima Life Sciences (Gencay, France). This electrochemical sensor measures the electron transfer between two conductive elements: the sensor and a solution of H₂O₂. The H₂O₂ solution is used as a marker for the determination of the detoxification power: as H₂O₂ is one of the most oxidizing non-radical species, any molecule capable of decreasing its concentration can be considered as an antioxidant. When a sample is in the presence of H₂O₂, all its antioxidative species spontaneously react with it and decrease its quantity. As the area of the peak measured by the sensor is directly proportional to H₂O₂ concentration, the detoxifying activity of a compound is evaluated by its modulation of the peak area.

H₂O₂ 5 mM in electrolytic ethanol solution was mixed with E4 in electrolytic ethanol solutions at several concentrations (0.16-0.5-1.6-5-16-50 mM), and the H₂O₂ oxidation peaks area were measured. The power of the antioxidant activity was calculated by comparing the peak area of the H₂0₂ solution with the peak area of the H₂O₂ solution mixed with an E4 solution.

A potent antioxidant activity of E4 following a positive dose-response was observed. When the molar ratio E4/H₂O₂ reached 1, the mean power was 77.5%, which was similar to the reference compound (EUK-134). A 100% antioxidant activity was observed for the highest E4 concentration (FIG. 8).

### Example 7: Healthy Female Donor Hair Follicle Organ Culture (Ex Vivo Model)

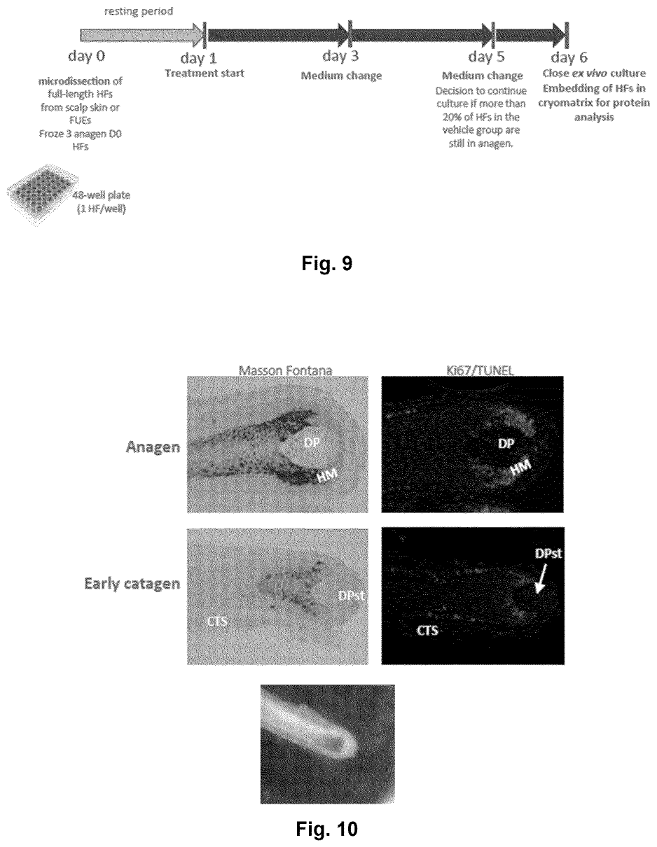

12-44 full-length frontotemporal hair follicles (HF) were obtained from four healthy human female donors >50 years old. Donors were caucasian female subjects of 59, 62, 59 and 67 years old, respectively (Donor 1:12 temporal anagen HFs; Donor 2:29; Donor 3:44; Donor 4:32). After microdissection from skin as described in the art (Edelkamp et al., Methods Mol Biol, 2020, 2154:105-119; and Langan et al., Exp Dermatol, 2015 December; 24(12):903-1), HFs were placed in adequate culture medium and allowed a 24 h rest period to allow them to recover from the trauma of microdissection. Test substance (either vehicle (0.3% EtOH) or E4 at concentrations of 30 μM, 3 μM or 300 nM) was added to the medium at the end of the 24 h. Total culturing time was 5-9 days depending on the stage of the HFs in the vehicle group. The experimental design for a 6-days HF organ culture experiment is depicted in FIG. 9.

This model allows to assess if E4 treatment is able to prolong anagen and/or to delay catagen initiation in healthy female subjects. Clinically, hair loss is associated with a reduction of HFs in anagen or with a shortening of anagen duration. Promoting anagen might thus limit hair loss. The following parameters were investigated:

Lactate Dehydrogenase (LDH) Release into the Medium

LDH is released from the cytoplasm of damaged cells into the culture medium. Therefore, the cytotoxicity of agents can be estimated based on the activity of LDH present in the culture medium using a colorimetric assay (Cytotoxicity Detection Kit [LDH], Roche) as described (Gherardini et al Int J Cosmet Sci, 2019 April; 41(2):164-182). The measurements were performed on supernatants collected on days respective to each culture and normalized to blank.

For the maximum release, HFs were treated with 1% Triton X 100 in vehicle medium at 37° C. for 30 minutes in order to induce maximum LDH release into the medium.

Hair Follicle Elongation (Correlation with Hair Shaft Production) Ex Vivo

To determine the HF length, each HF was measured from the end of the bulb connective tissue sheath to the end of the distal outer root sheath (ORS), using a digital light microscope at 50× magnification (VHX 900 Keyence Corporation, Osaka, Japan) and the affiliated software (Edelkamp et al., Methods Mol Biol, 2020, 2154:105-119; and Langan et al., Exp Dermatol, 2015 December; 24(12):903-1). Measurements were obtained at baseline, after 24 h rest period and subsequently every 24-48 h during the entire duration of the experiment. Elongation is expressed as millimeters of growth and/or as percentage elongation from the baseline measurement.

### Microscopic Hair Cycle Staging and Scoring

Hair cycle staging and calculation of hair cycle score is indicative of hair cycle progression and anagen-catagen transition (Langan et al., Exp Dermatol, 2015). Hair cycle staging and hair cycle scoring was performed at the end of the culture. The hair cycle stage of each HF was determined according to several established macroscopic and microscopic parameters (Langan et al Exp Dermatol, 2015). Microscopic hair cycle staging was determined using Ki-67/TUNEL immunohistology and Masson-Fontana histochemistry. Hair cycle staging was analyzed by calculating the percentage of HFs in each hair cycle stage. Hair cycle score was determined by giving an arbitrary score to each hair cycle stage as following: anagen: 100, early catagen: 200, mid catagen: 300, late catagen: 400, dystrophic 500.

| Hair cycle | | | | Late | Dystrophic |
|---|---|---|---|---|---|
| stage | Anagen | Early catagen | Mid catagen | catagen | (anagen/catagen) |
| Hair bulb | Rounded | Bullet-like | Slightly | Thinned | Swollen bulb, |
| shape | | | triangulated, | and | separation of the |
| | | | swelling | elongated | epithelial from the |
| | | | | | mesenchymal |
| | | | | | tissue, spongiosis |
| | | | | | in the ORS |
| Hair matrix | Thick, | Thinner, | Very thin, | Very thin, | As anagen or |
| shape and | closed hair | opening of hair | shortened | slightly | catagen |
| thickness | matrix | matrix | | withdrawn | |
| Dermal | Almond- | More rounded | Densely | Very | As anagen or |
| papilla (DP) | shaped | | packed, and | densely | catagen |
| shape | | | rounded | packed and | |
| | | | | rounded | |
| Accumulation | no | yes | yes | yes | As anagen or |
| fibroblasts in | | | | | catagen |
| the dermal | | | | | |
| papilla stalk | | | | | |
| Pigmentation | Very | Less | Less | Less | Appearance of |
| | pigmented, | pigmented, | pigmented, | pigmented, | melanin clumping |
| | starting at | starting at the | starling | starling at | |
| | the level of | level of | above | the level of | |
| | Auber's | Auber's line or | dermal | club hair | |
| | line and | above dermal | papilla | | |
| | expand to | papilla | | | |
| | half of hair | | | | |
| | matrix | | | | |
| Germinative | Prominent | Decrease in | No | No | As anagen or |
| hair matrix | below the | the number of | proliferation | proliferation | catagen |
| proliferation | Auber's | proliferating | below the | below the | |
| | line | cells below the | Auber's line | Auber's line | |
| | | Auber's line | | | |
| Hair matrix | No | Little | Apoptosis in | Apoptosis in | As anagen or |
| apoptosis | apoptosis | apoptosis may | the hair | the | catagen |
| | in the hair | be observed in | matrix and | epithelial | |
| | matrix | the hair matrix | epithelial | strand | |
| | | | strand | | |

The anagen and early catagen phases, as well as a representative image of a dystrophic hair follicle are depicted in FIG. 10.

### Hair Matrix Keratinocytes Proliferation and Apoptosis

Germinative hair matrix keratinocytes proliferate mostly during anagen. Their proliferation is dramatically reduced during the anagen/catagen transition, and absent in late catagen (Langan et al., Exp Dermatol, 2015). Apoptosis of hair matrix keratinocytes is used as an additional indicator of anagen/catagen transition.

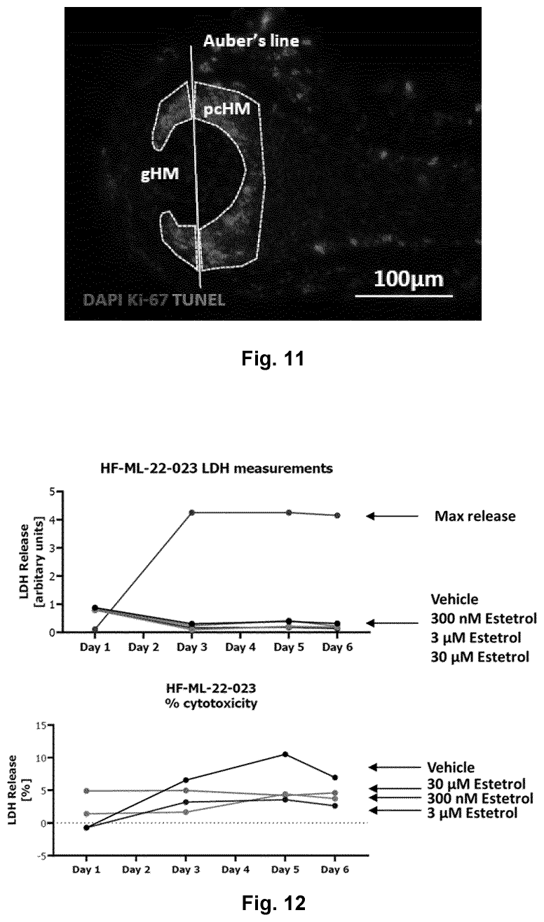

Proliferation was assessed by quantification of the percentage of hair matrix keratinocytes in a clearly specified area (germinative hair matrix keratinocytes, below the Auber's line) that were positive for the proliferation marker Ki67, whereas apoptosis was assessed using TUNEL (TdT-mediated dUTP-biotin nick-end labelling) immunofluorescence in the same keratinocyte population (below the Auber's line) and in the precortical hair matrix keratincoytes (above the Auber's line) (Langan et al., Exp Dermatol 2015) (FIG. 11).

### Alkaline Phosphatase Activity-Dermal Papilla (DP) Inductivity

Alkaline phosphatase is an indicator of DP inductivity (Yang, J Dermatol Sci, 2010 January; 57(1):2-11). Alkaline phosphatase in situ activity was measured in the DP.

### Versican Expression-DP Inductivity

Versican, a large chondroitin sulfate proteoglycan molecule, is expressed in DP in anagen HFs and diminishes in catagen HFs and its expression correlates with hair inductive properties (Yang, J Dermatol Sci 2010). Versican intensity was analyzed in the DP.

### CD34 Expression-Stem Cell Progeny

CD34 is a marker expressed by the progeny of HF bulge stem cells. It is expressed in the most external layer of the outer root sheet (ORS) below the isthmus (sub-bulge to suprabulbar regions) (Purba et al., BioEssays, 2014 May; 36(5):513-25). CD34 expression and percentage of CD34⁺ cells were analyzed in the suprabulbar ORS.

### DP Fibroblast Emigration.

The DP resident fibroblasts are subjected to migration in the transition between anagen and catagen phase (Hawkshaw et al., Journ of Invest Dermatol, 2015, 135(8):2129-2132). In catagen onset, DP fibroblasts emigrate to the dermal cup through the DP stalk, whereas at anagen induction, they immigrate back in the DP. Quantifying the number of DP fibroblasts present in the DP stalk and in the DP (Kloepper et al., Exp Dermatol, 2010 March; 19(3):305-12) is therefore a direct readout for anagen induction or catagen promotion.

### K15 Expression—Stem Cells.

K15 is a type I cytokeratin expressed in HF bulge stem cells and their progeny (Purba et al., BioEssays, 2014). It is expressed in the bulge, infra-bulge and suprabulbar ORS. Bulge stem cells are identified using K15 expression, cellular morphology and anatomical localization (below the sebaceous glands, at the area where the arrector pili muscle attaches to the HF). K15 expression was analyzed in the bulge basal layer and in the suprabulbar ORS, and proliferation of K15 positive cells was evaluated in combination with Ki67 immunofluorescence staining.

### Statistics

Each dot represents a single hair follicle; Meant SEM; GraphPad Prism 9; D'Agostino & Pearson omnibus normality test; for group comparisons (#p<0.05, ##p<0.01, ###p<0.001, ####p<0.0001), non-Gaussian distribution (Kruskal-Wallis test, Dunn's multiple comparison test—each group vs vehicle), Gaussian distribution (One-way ANOVA, Dunett's or Holm-Sidak's multiple comparison test—each group vs vehicle); pairwise comparisons (* p<0.05), no Gaussian distribution (Mann-Whitney), Gaussian distribution (t test); ns, not significant. Statistics for non-Gaussian distribution were followed if at least one group did not show a Gaussian distribution within a given group or pair-wise comparison.

n=3-4 donors, individual HFs. Data were first analysed for Gaussian distribution using D'Agostino & Pearson omnibus normality test and outliers were identified and removed using ROUT method (0=10/o). When data followed Gaussian distribution, we performed one-way ANOVA analysis followed by Holm-Sidak's multiple comparison test—vehicle fixed and Unpaired student's t-test versus vehicle. When data were too small to determine whether the data follow Gaussian distribution or data did not follow Gaussian distribution, we performed Kruskal-Wallis test followed by Dunn's multiple comparison test—vehicle fixed and Mann-Whitney test versus vehicle. Note that the only anagen evaluations include only the data of n=3 patients. Donor 1 has no anagen HFs in the vehicle group.

### Results

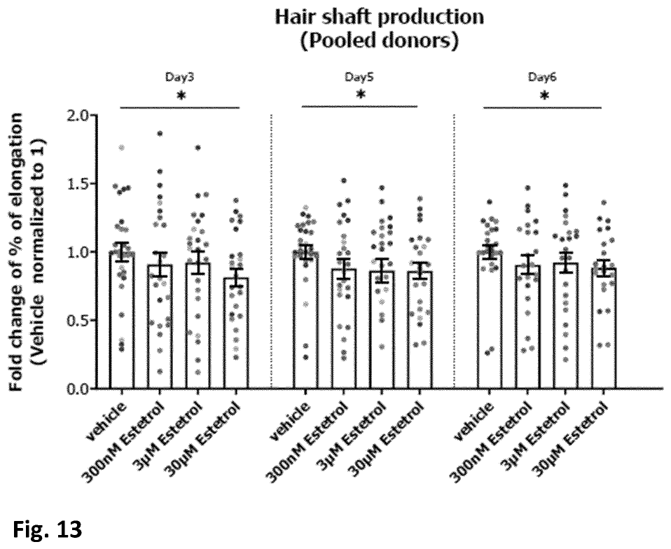

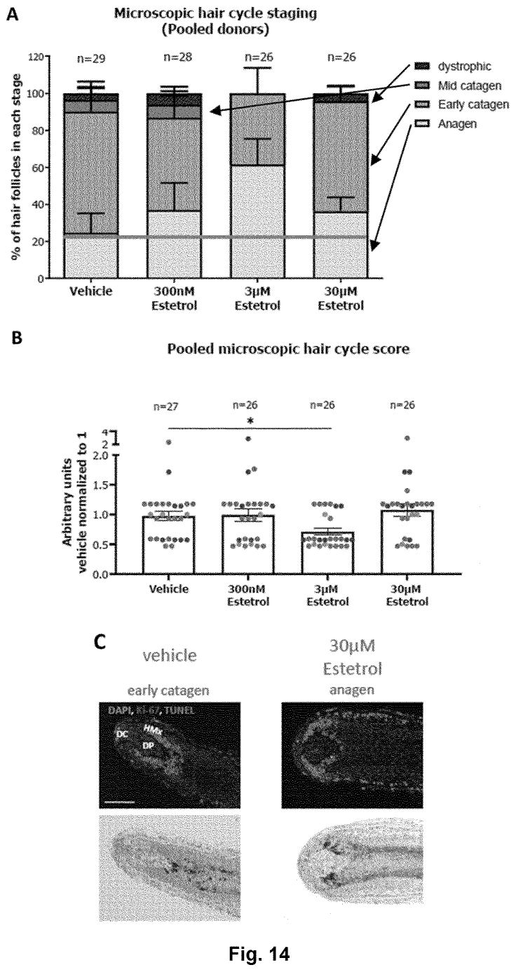

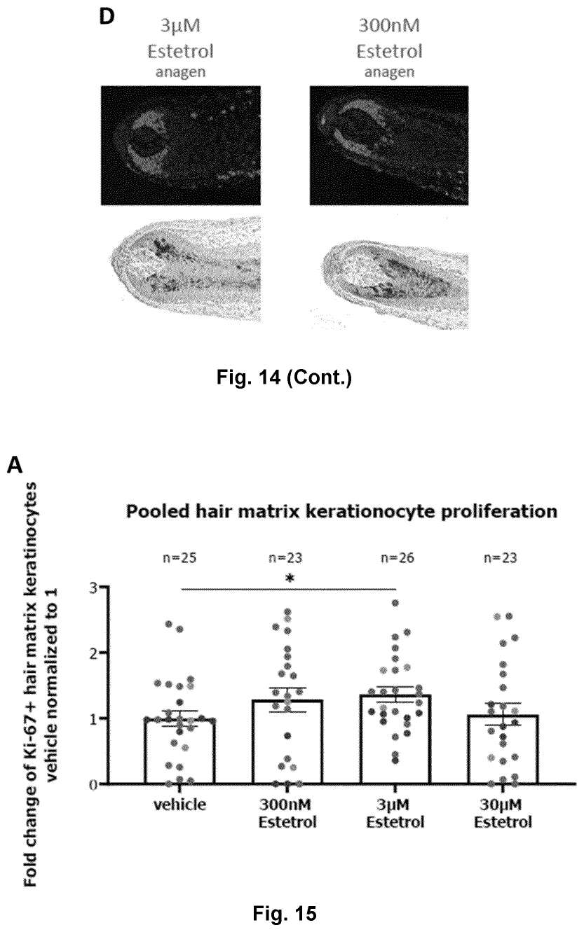

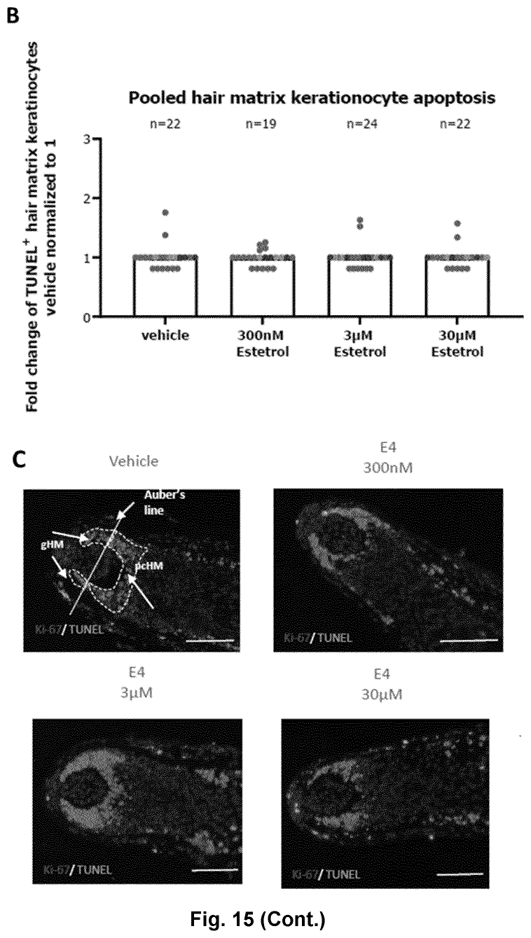

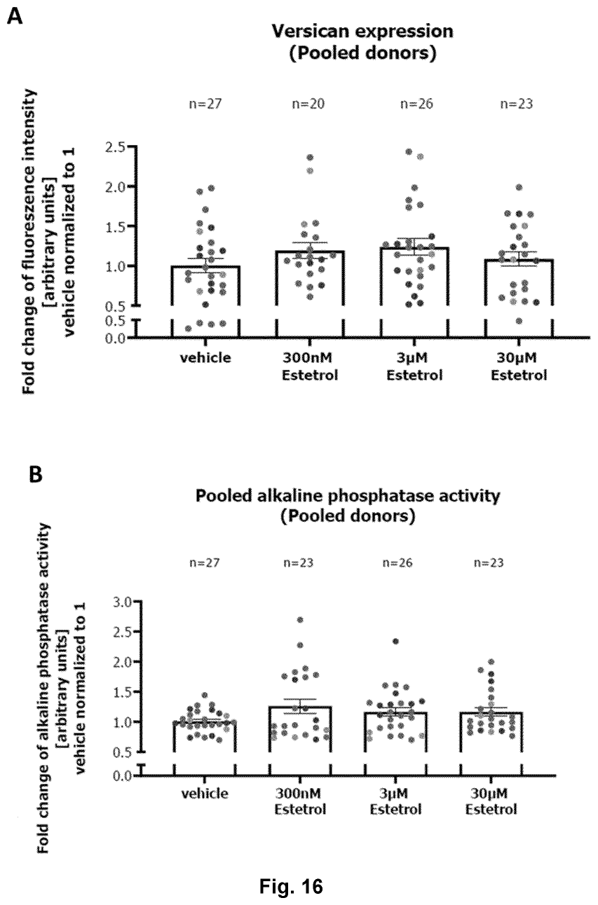

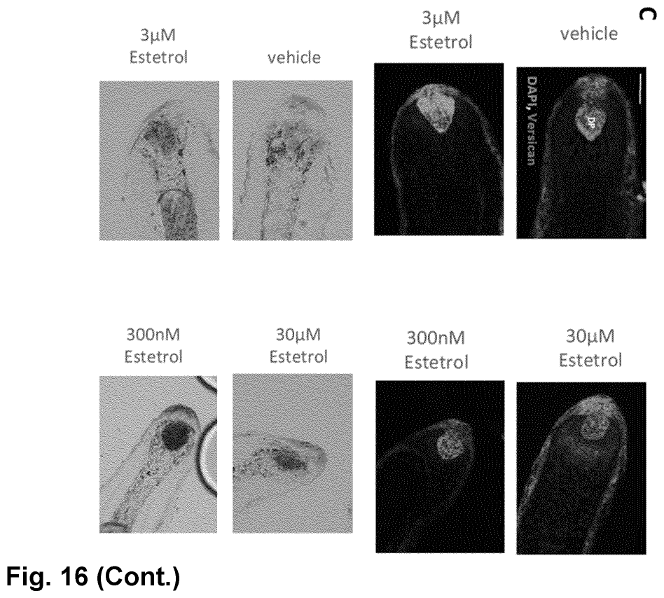

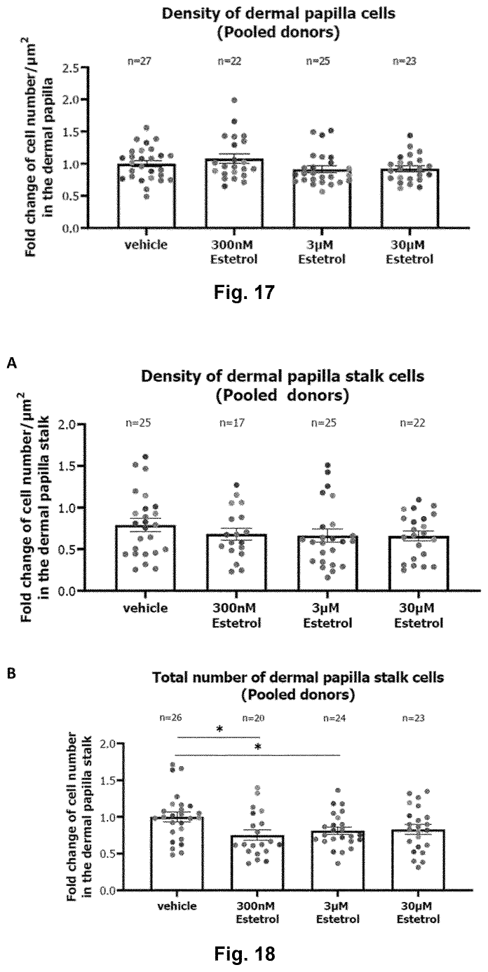

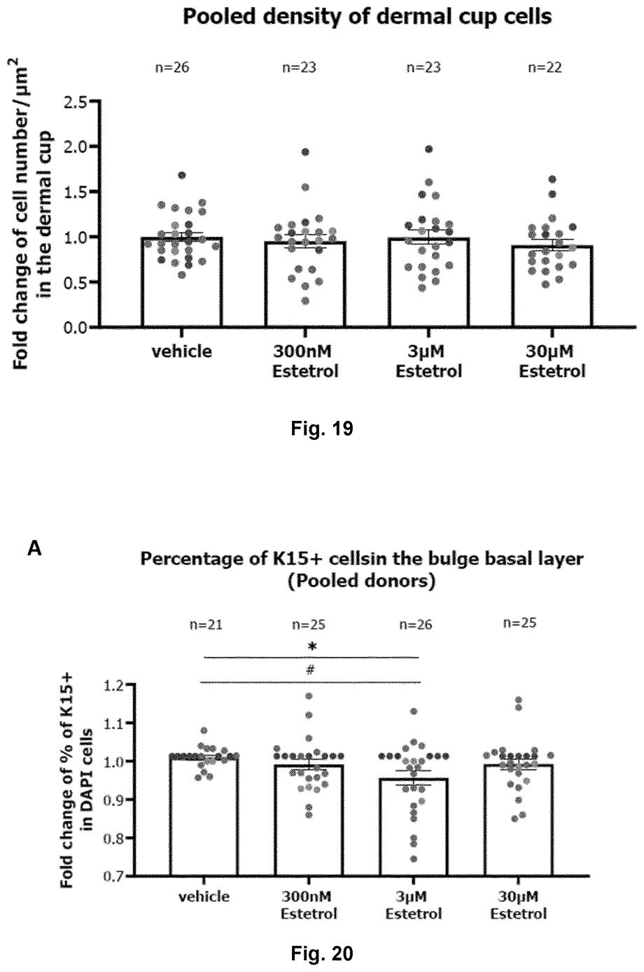

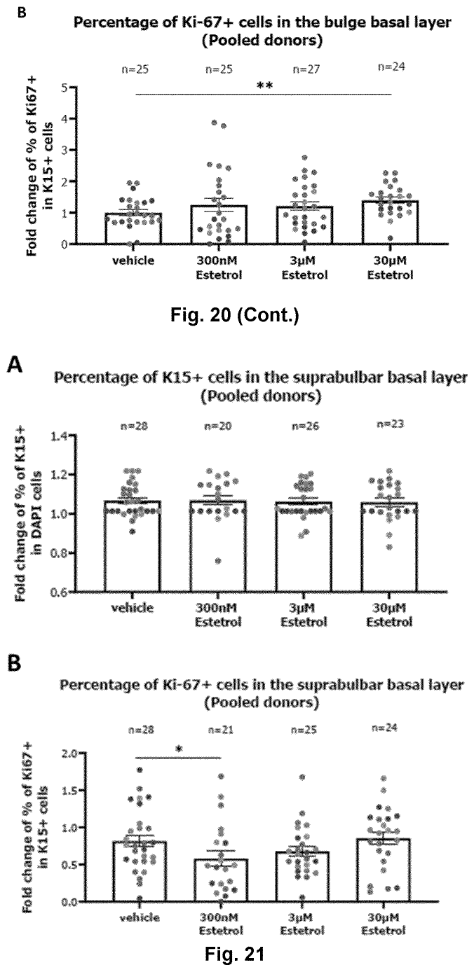

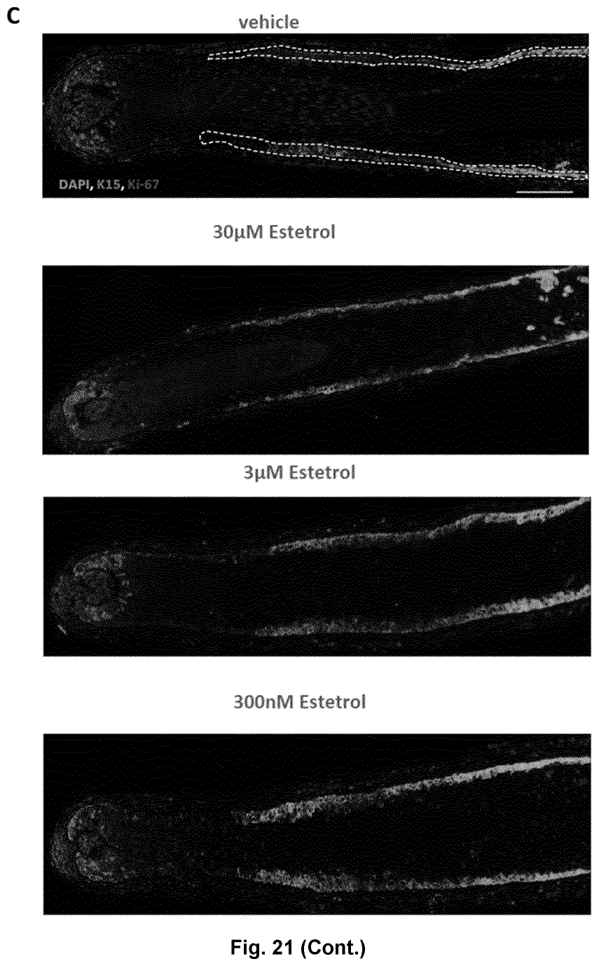

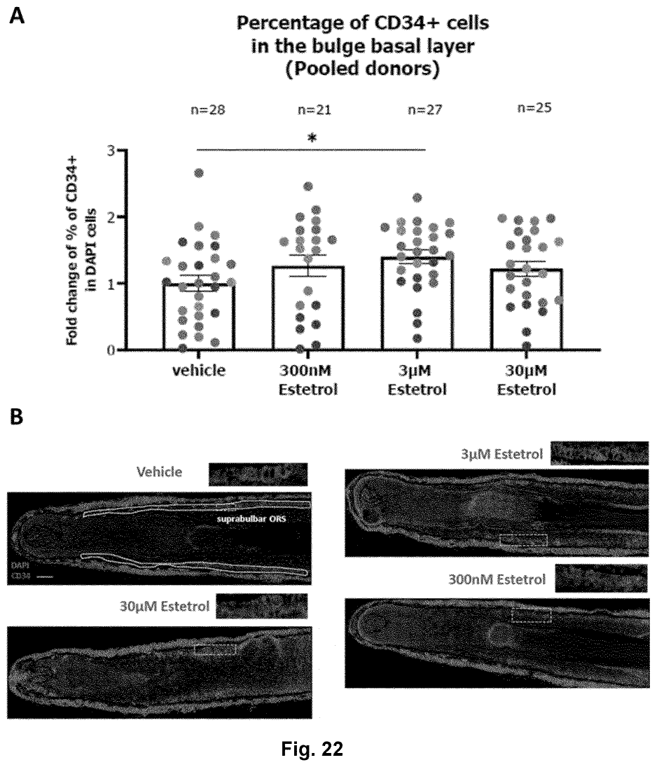

All tested concentrations of E4 did not induce HF cytotoxicity (LDH release into the medium) (FIG. 12).
E4 did not modify hair shaft production (FIG. 13)
Microscopically, E4 3 μM (significantly) and 300 nM and 30 μM (tendentially) prolonged anagen (FIG. 14).
E4 3 μM (significantly) and 300 nM and 30 μM (tendentially) increased hair matrix keratinocytes proliferation, without affecting hair matrix keratinocytes apoptosis (FIG. 15).
All tested concentrations of E4 tendentially increased versican expression and alkaline phosphatase activity in DP cells (FIG. 16).
DP fibroblast emigration analysis revealed that all tested concentrations of E4 did not strikingly affect the density of cells in the DP (FIG. 17), all concentrations tendentially decreased the density of cells and 300 nM and 3 μM E4 significantly reduced the total number of cells in the DP stalk (FIG. 18), whereas E4 had no significant impact on the density of cells in the inductive dermal cup (FIG. 19).
In the bulge basal layer, E4 3 μM significantly decreased the percentage of K15 positive cells, whilst E4 30 μM (significantly) and 3 μM and 300 nM (tendentially) increased their proliferation (FIG. 20).
In the suprabulbar ORS, E4 did not have any effect on the percentage of K15 positive cells, but E4 300 nM (significantly) and 3 μM (tendentially) decreased their proliferation (FIG. 21).
E4 3 μM (significantly) and 300 nM and 30 μM (tendentially) increased the percentage of CD34 positive cells in the suprabulbar ORS (FIG. 22).

### Discussion and Conclusions

The experiments reported here investigate the potential effect of the estrogenic compound E4 on hair cycle, hair matrix keratinocytes, DP inductivity and DP fibroblasts emigration, stem cell maintenance, and generation of progeny in the context of hair growth promotion in healthy female subjects. The experiments confirmed that the tested concentrations of E4 did not induce HF cytotoxicity (LDH release in to the medium). Most importantly, the data from four individual donors revealed that E4, tendentially (300 nM and 30 μM) and significantly (3 μM), prolonged anagen ex vivo. The significant anagen prolonging effect of 3 μM E4 was further supported by the decrease in the density of cells in the DP stalk, as DP fibroblasts accumulate in this specific compartment during their emigration from the DP in catagen (Kloepper et al., Exp Dermatol. 2010). The anagen prolonging effect was also supported by a significant increase of hair matrix keratinocytes proliferation after the treatment with 3 μM E4.

The data suggested that the anagen prolonging effect of E4 may have been at least in part mediated by stimulation of DP fibroblast inductivity. Indeed, all tested concentrations of E4 seemed to tendentially upregulate alkaline phosphatase activity and versican expression in the DP.

In the bulge, 3 μM E4 seemed to tendentially increase the proliferation rate of pluripotent HF stem cells, whereas it significantly reduced the percentage of K15 positive cells. However, the treatment beneficially affected the generation and/or activities of the stem cell progeny in the suprabulbar ORS. Specifically, 3 μM E4 tendentially decreased the number of proliferative K15 positive cells and significantly increased the percentage of CD34 positive cells, the latter also further corroborating the anagen prolongation effect of E4 (Purba et al., 2014).

Taken together, these experiments further encourage the exploration of 3 μM E4 treatment for hair growth promotion in healthy female subjects. E4 may have the potential to maintain anagen and possibly prevent HFs from miniaturization. The above experiments can be repeated for distinct subject groups (healthy males, female pattern hair loss subjects and male pattern hair loss subjects) in Examples 8 to 10.

### Example 8: Healthy Male Donor Hair Follicle Organ Culture (Ex Vivo Model)

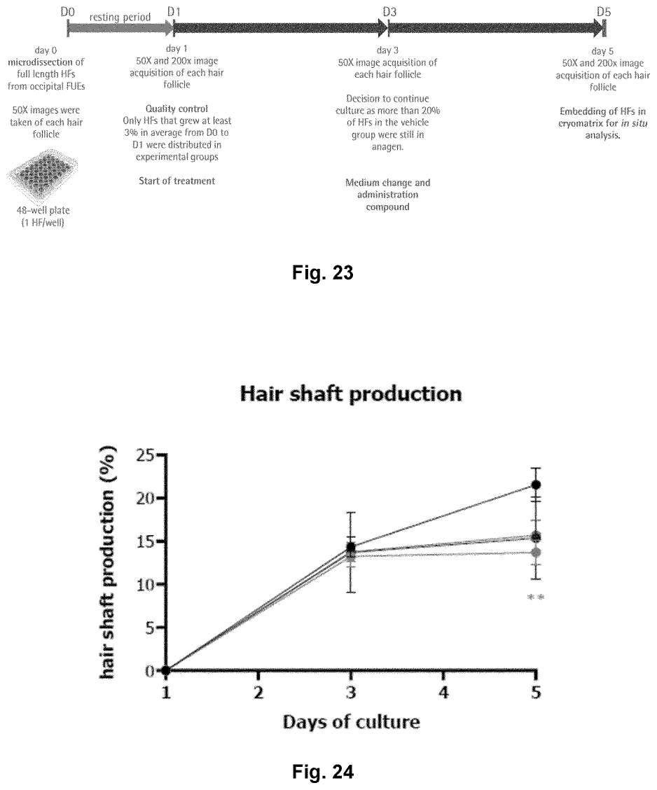

Example 7 is repeated for one healthy male donor subject, with a few minor modifications. In brief, 24 full-length occipital hair follicles (HF) were obtained from one healthy Caucasian male donor of 47 years old. After microdissection from skin as described in the art (Edelkamp et al., Methods Mol Biol, 2020, 2154:105-119; and Langan et al., Exp Dermatol, 2015 December; 24(12):903-1), HFs were placed in adequate culture medium and allowed a 24 h rest period to allow them to recover from the trauma of microdissection. Test substance (either vehicle (0.03% EtOH), E2 at concentration of 300 nM or E4 at concentrations of 3 μM or 300 nM) was added to the medium at the end of the 24 h. Total culturing time was 5 days depending on the stage of the HFs in the vehicle group. The experimental design is depicted in FIG. 23. The same parameters as those described in Example 7 were investigated.

### Results

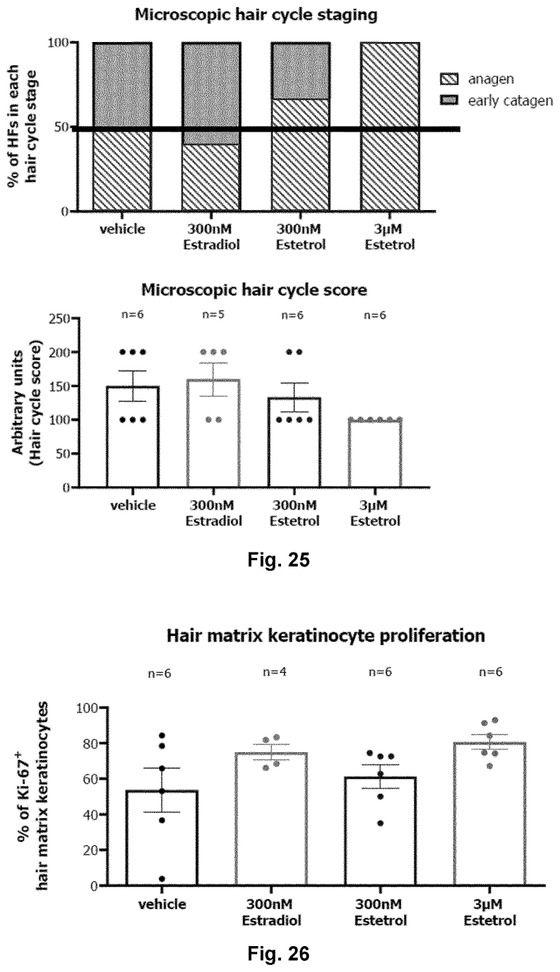

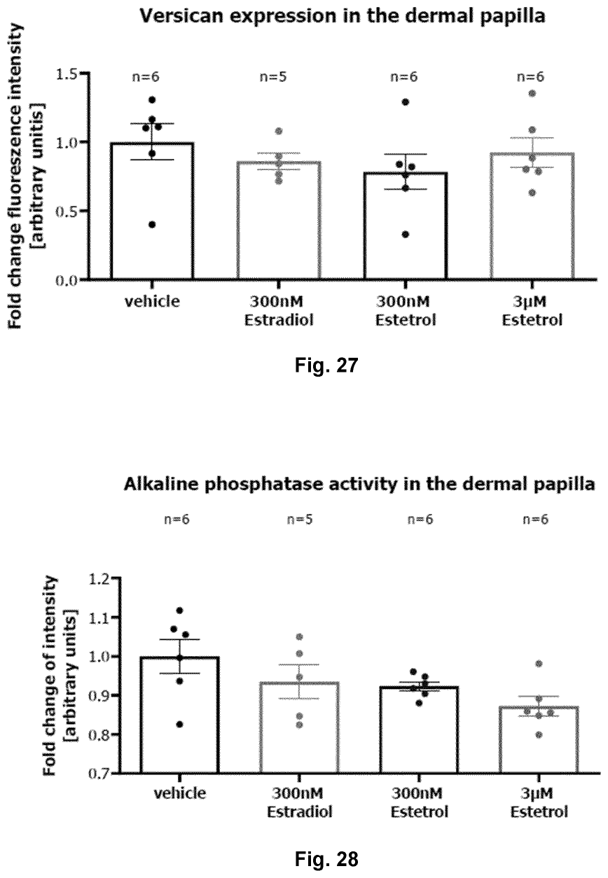

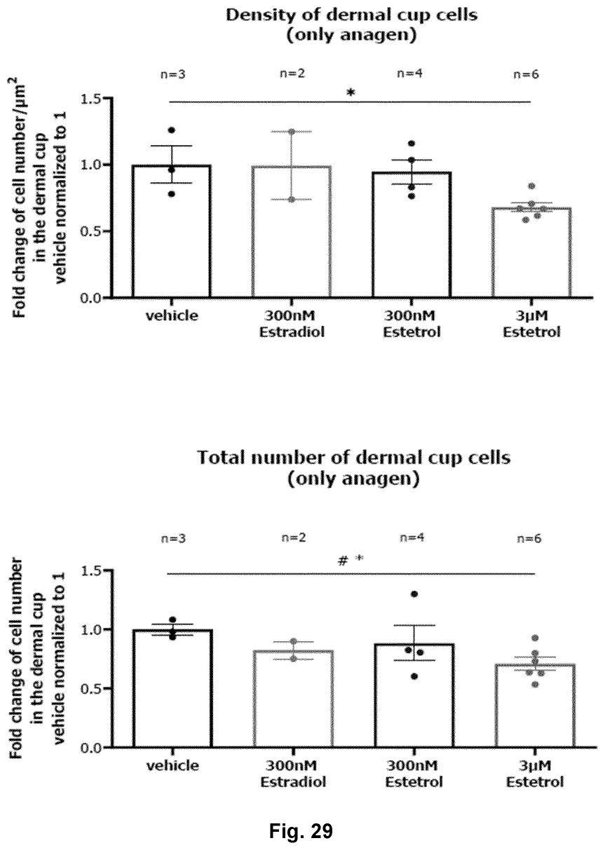

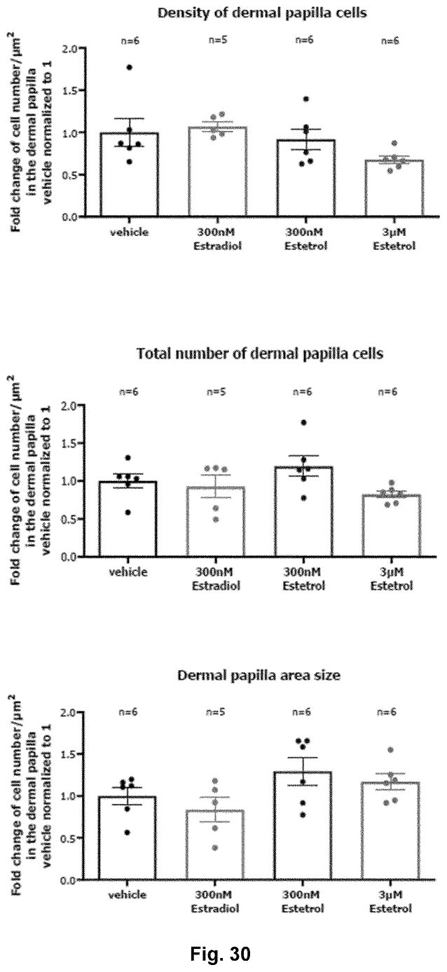

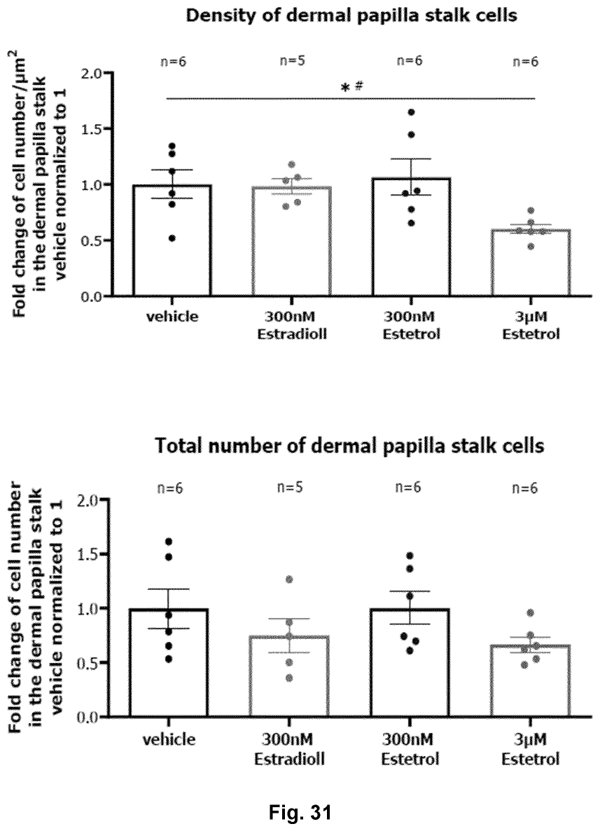

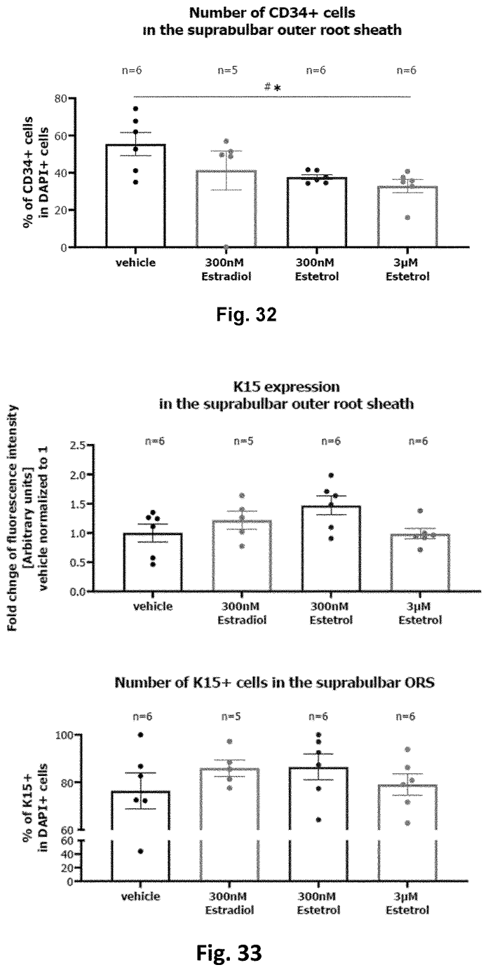

Hair shaft production was significantly reduced by E2 and tendentially by E4 300 nM and 3 μM (FIG. 24)
Microscopically, E4 maintained HFs in anagen, even more prominently at 3 μM, while E2 did not affect the hair cycle (FIG. 25)
Hair matrix keratinocyte proliferation was tendentially increased by E4 3 μM and E2 (FIG. 26)
Versican expression and alkaline phosphatase activity in the dermal papilla seemed to be tendentially decreased by all treatments (FIGS. 27 and 28)
The density and total number of cells in the dermal cup of anagen HFs were significantly decreased by E4 3 μM, while E2 had no effect (FIG. 29)
The density and total number of cells in the dermal papilla were tendentially decreased by E4 3 μM and its area was increased by E4 300 nM and 3 μM, while E2 had no effect (FIG. 30)
The density and total number of cells in the dermal papilla stalk were decreased by E4 3 μM, while E2 had no effect (FIG. 31)
The number of CD34+ cells in the suprabulbar outer root sheath was decreased by E4 300 nM and 3 μM, while E2 had no effect (FIG. 32)
K15 expression and number of cells in the suprabulbar outer root sheath were tendentially increased by E4 300 nM and E2 (FIG. 33)

### Discussion and Conclusions

The goal of the experiment was to confirm whether the findings related to HFs of healthy female subjects could be extrapolated to HF of healthy male subjects and thus provide a motivation to further explore estetrol treatment for hair growth promotion in healthy male subjects.

This experiment confirmed the anagen prolonging effect of E4 ex vivo in male HFs for all tested concentrations as was previously observed in healthy female HFs. However, E2 at 300 nM did not display any effect in this specific donor.

The encouraging anagen prolonging effect of E4 3 μM was further supported by the significant reduction of cells accumulating in the dermal papilla stalk, as fibroblasts accumulate in this specific compartment during their emigration from the dermal papilla into the dermal cup as a consequence of catagen transition (Kloepper et al., Exp Dermatol, 2010 March; 19(3):305-12). Furthermore, the increased proliferation of hair matrix keratinocytes with E4 3 μM also supported the observed anagen prolonging effect.

Altogether, these results are promising and further encourage the exploration of E4 treatment in additional male donors. E4 is suggested to have the potential to maintain anagen and to prevent HFs from miniaturization, and should be envisaged for male pattern hair loss management.

### Example 9: Female Pattern Hair Loss (FPHL) Donor Hair Follicle Organ Culture (Ex Vivo Model)

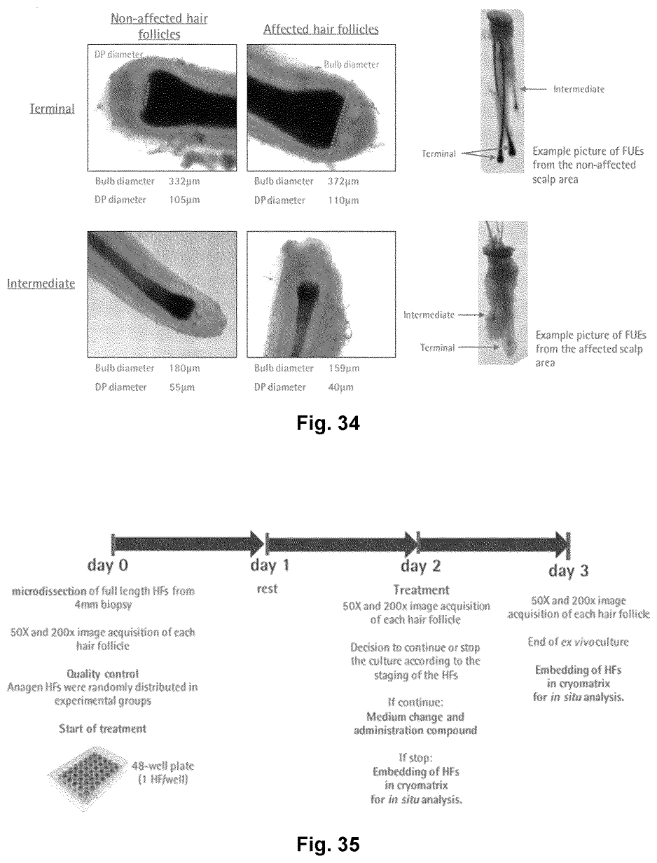

Example 7 is repeated for female donor subjects suffering from pattern hair loss (FPHL), with a few modifications. In brief, terminal and intermediate hair follicles (HF) from affected (vertex) and non-affected (occipital) areas were obtained from female donors diagnosed to have FPHL (FIG. 34). After microdissection from skin as described in the art (Edelkamp et al., Methods Mol Biol, 2020, 2154:105-119; and Langan et al., Exp Dermatol, 2015 December; 24(12):903-1), HFs are placed in adequate culture medium and allowed a 24 h rest period to allow them to recover from the trauma of microdissection. Test substance (either vehicle (0.03% EtOH) or E4 at concentrations of 3 μM) was added to the medium at the end of the 24 h. Total culturing time was 3-5 days depending on the stage of the HFs in the vehicle group. The experimental design is depicted in FIG. 35. The same parameters as those described in Example 7 were investigated.

Preliminary results are available for two donors, one Caucasian female of 60 years with diffuse FPHL pattern and one middle eastern female of 31 years with classical FPHL pattern. Three additional donors still have to be collected.

### Preliminary Results

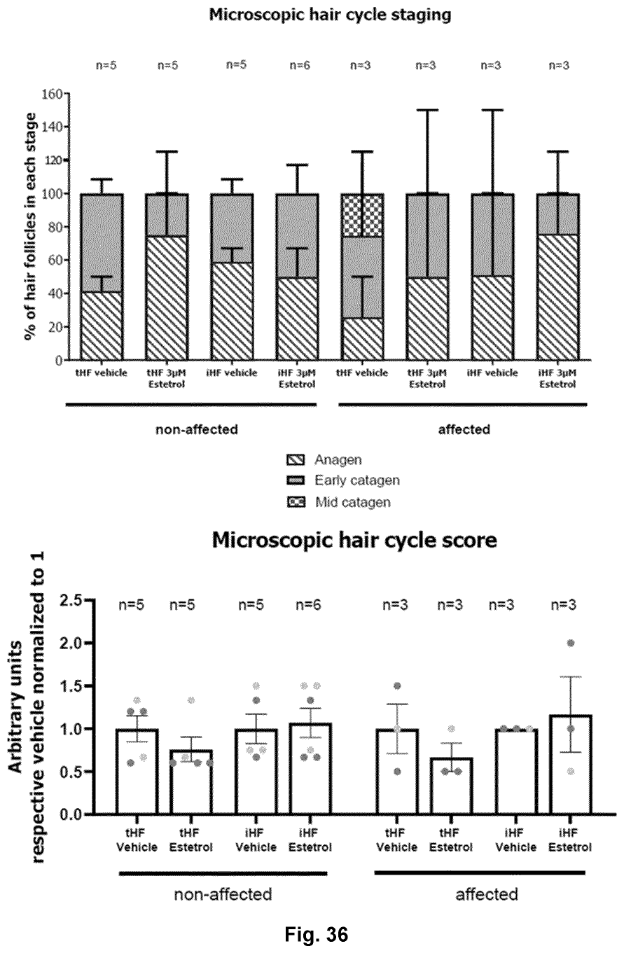

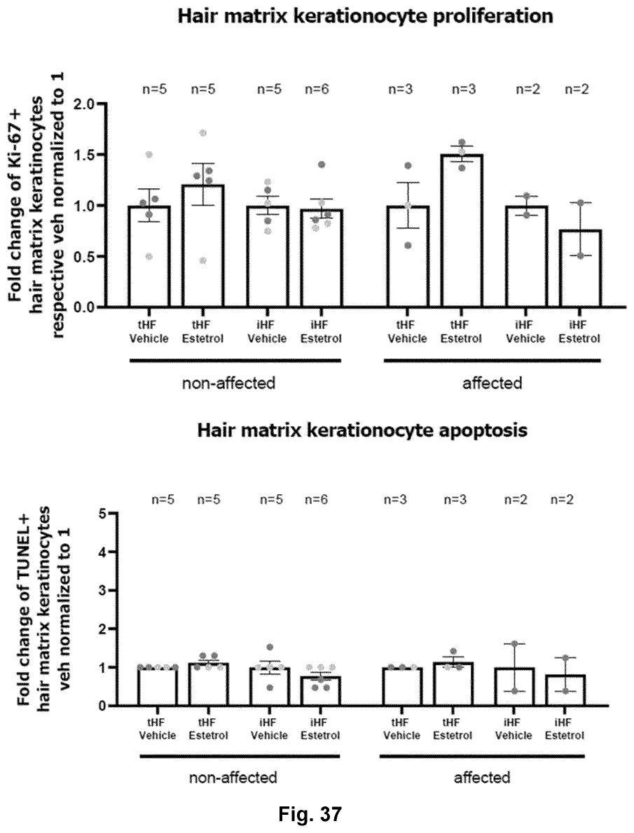

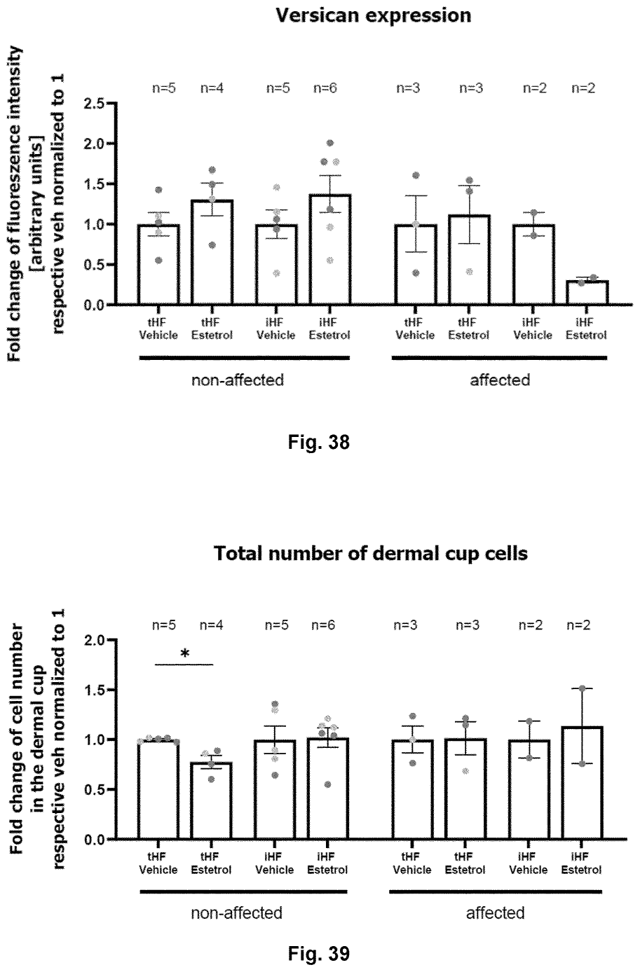

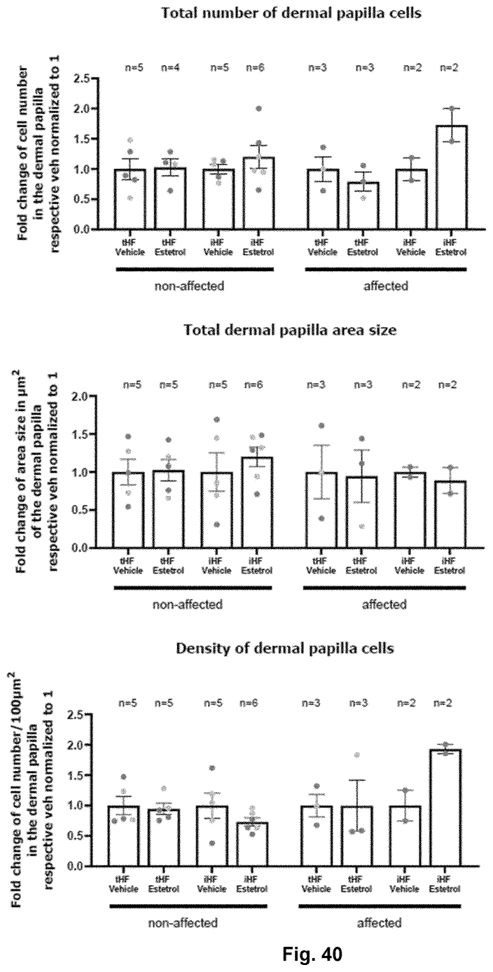

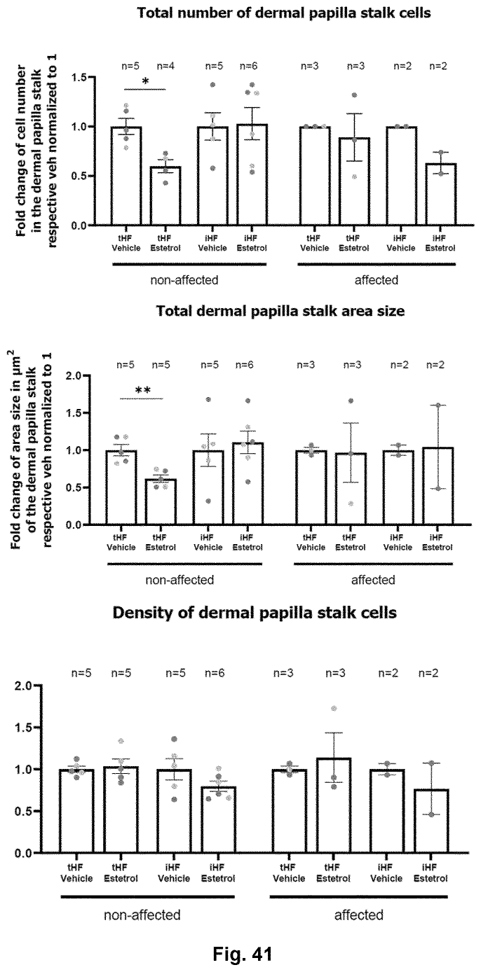

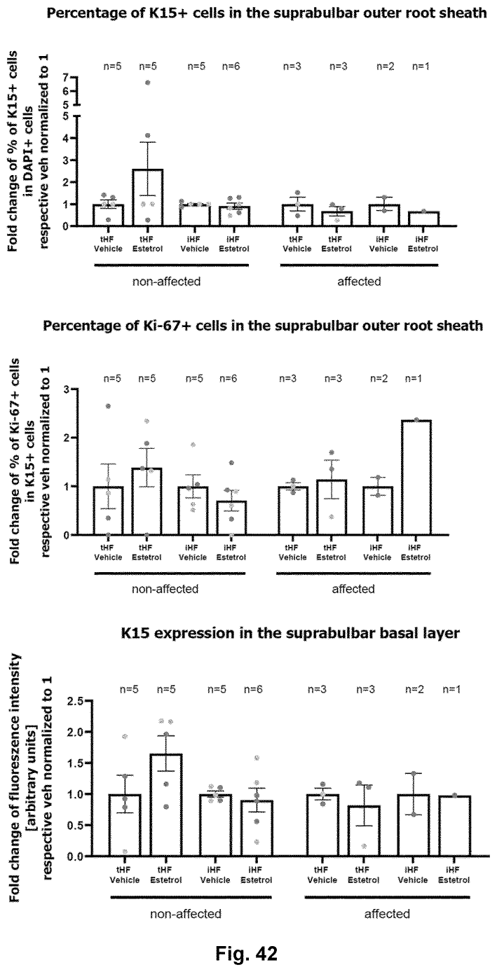

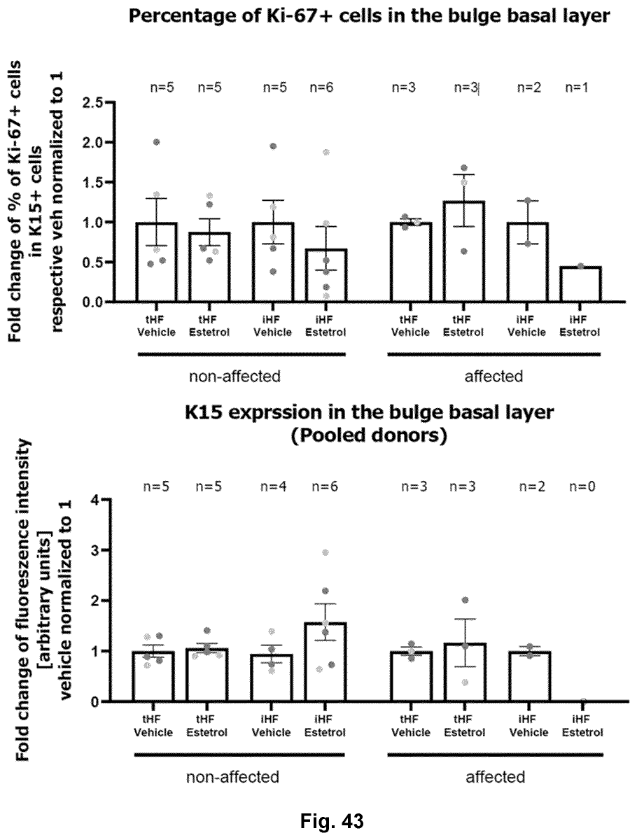

Microscopically, E4 prolonged anagen in HFs from non-affected (terminal) and affected (terminal and intermediate) scalp (FIG. 36)
E4 tendentially increased hair matrix keratinocytes proliferation in terminal HFs from non-affected and affected scalp areas, without affecting hair matrix keratinocytes apoptosis (FIG. 37)
E4 tendentially increased versican expression in terminal and intermediate HFs from non-affected scalp (FIG. 38)
E4 significantly reduced the total number of cells in the dermal cup of terminal HFs from the non-affected scalp (FIG. 39)
E4 tendentially increased the total number of cells and area size of the dermal papilla in intermediate HFs from the non-affected scalp, and tendentially increased the density and total number of cells in intermediate HFs from the affected scalp (FIG. 40)
E4 significantly decreased the total number of cells and area size of the dermal papilla stalk in terminal HFs from the non-affected scalp, and tendentially decreased the density of dermal papilla stalk cells in intermediate HFs from the non-affected scalp (FIG. 41)
In the suprabulbar outer root sheath, E4 tendentially increased K15 expression and number of positive cells in terminal HFs from the non-affected scalp, and increased proliferation of K15+ cells in terminal HFs from non-affected and intermediate HFs from affected scalp (FIG. 42)
In the bulge basal layer, E4 tendentially increased K15 expression in intermediate HFs from the non-affected scalp, and increased proliferation of K15+ cells in terminal HFs from affected scalp (FIG. 43)

### Discussion and Conclusions

The goal of the experiment was to confirm that the findings relating to HFs of healthy female subjects, as well as healthy male subjects, could be extrapolated to HF of female subjects affected by FPHL and thus provide a motivation to further explore estetrol treatment for hair growth promotion in female subjects confronted with hair loss.

Preliminary data from two individual patients confirmed the anagen prolonging effect of E4 on terminal HFs from the non-affected scalp, in line with an increase in hair matrix keratinocytes. This beneficial effect was supported by a significant reduction in the number of cells in the dermal papilla stalk and dermal cup, as fibroblasts accumulate in these specific compartments during their emigration from the dermal papilla into the dermal cup as a consequence of catagen transition (Kloepper et al., 2010). Interestingly, E4 also seemed to maintain anagen in terminal and to a lesser extend intermediate HFs from affected area, whilst increasing the hair matrix keratinocytes only in terminal HFs.

Furthermore, E4 exerted an increase in dermal papilla inductivity in both terminal and intermediate HFs from non-affected area, through a tendential increase in versican expression and alkaline phosphatase activity, whilst HFs from affected area were not strikingly altered.

In addition, K15 expression and number of positive cells in terminal HFs from the non-affected area were tendentially increased by E4, along with an increase in their proliferation.

Taken together, the beneficial effect of E4 already seen in healthy female HFs could be recapitulated in terminal HFs from non-affected area and partially in terminal HFs from affected area. As the latter one corresponds to the HFs that are more prone to miniaturize, the preliminary data further encourage the exploration of E4 treatment for FPHL management, as E4 has the potential to maintain anagen and to prevent HFs from miniaturization.

### Example 10: Male Pattern Hair Loss (MPHL) Donor Hair Follicle Organ Culture (Ex Vivo Model)

Example 7 is repeated for male donor subjects suffering from pattern hair loss (MPHL). In brief, full-length frontotemporal hair follicles (HF) were obtained from Caucasian male donors diagnosed to have MPHL. After microdissection from skin as described in the art (Edelkamp et al., Methods Mol Biol, 2020, 2154:105-119; and Langan et al., Exp Dermatol, 2015 December; 24(12):903-1), HFs are placed in adequate culture medium and allowed a 24 h rest period to allow them to recover from the trauma of microdissection. Test substance (either vehicle (0.3% EtOH) or E4 at concentrations of 30 μM, 3 μM or 300 nM) is added to the medium at the end of the 24 h. Total culturing time is 5-9 days depending on the stage of the HFs in the vehicle group. This model allows to assess if E4 treatment is able to prolong anagen and/or to delay catagen initiation in male subjects affected by MPHL. The same parameters as those described in Example 7 are investigated.

The experiment intends to confirm that the findings relating to HFs of healthy female and male subjects, as well as female subjects affected by FPHL, can be extrapolated to HF of male subjects affected by MPHL and thus provide a motivation to further explore estetrol treatment for hair growth promotion in male subjects confronted with hair loss.

Preliminary results described in example 8 are promising and further encourage this exploration.

### Example 11: A Clinical Study to Assess the Effect of Estetrol Treatment in Menopausal (Peri- and Post-Menopausal) Woman on Hair Loss

A randomized, double-blind, placebo-controlled, phase II (pilot) study to evaluate the effects of estetrol in postmenopausal women with androgenetic alopecia and to assess the influence on hair parameters is started.

### Study Design:

This randomized, double-blind, placebo-controlled, phase II (pilot) study consists of a dermatological part.

The determination of subject eligibility includes a classification of pattern of hair loss by using the Savin Density Scale. Subjects will be randomly allocated and will receive either a topical foam formulation based on estetrol monohydrate (0.06%, 0.22% or 0.50% respectively formulated as a foam using butane, butylated hydroxytoluene, cetyl alcohol, citric acid, glycerol, isobutane, lactic acid, polysorbate 60, propane, purified water, stearyl alcohol and ethanol anhydrous) or placebo topical foam formulation. Note that due to solubilisation during the formulation process the estetrol monohydrate may no longer be present in the monohydrate form, which should be evaluated for each topical composition separately.

Dermatological investigations include quantitative hair measurements, Subject Self-Assessment (SSA), and Investigator's Global Assessment (IGA) of hair growth. Safety of estetrol (E4) is assessed throughout the study in all subjects.

### Number of Participants (Planned:

### 120 Participants (30 Per Treatment Regime)

This sample size has been determined for a 5% two-sided type I error and a power of 80% to detect a 20-hairs/cm² difference between groups in Target Area Hair Count (TAHC), assuming a standard deviation of 25 hairs/cm², considering the use of non-parametric approach (Sauerbronn, int J Gynaecol Obstet, 2000 January; 68(1):35-41).

### Dermatological Efficacy Variables:

Dermatological measurements and assessments are performed at baseline (either at Screening or at the following visit) and at visits (e.g. week 12 and 24; month 4, 8 and 12).

Primary dermatological efficacy variables will be:
Target Area Hair Count (TAHC),
Hair Growth Assessment (HGA).

Other variables will be considered as secondary variables.

Hair parameters will be assessed using the following measurements:

Digital image analysis from standardized Macro Photography (HairMetrix Phototrichogram, Canfield) for quantitative hair measurements:

Target Area Hair Count (TAHC) and

Target Area Hair Width (TAHW) for non-vellus and vellus/vellus like (miniaturized) hair, separately. A scalp micro dot tattoo is performed at baseline and during one or more of the visits to assist with standardized macro photography.

Standardized Global Photography for SSA and IGA using the global photo of the subject's scalp taken at baseline through the Canfield Camera device software and comparing it to the respective real time global photo made instantly available in the Canfield review software installed on the Canfield supplied laptop

Subject self-assessment of scalp hair growth will be performed by the following 3 assessments: Hair Growth Assessment (HGA) (Olsen E A, Whiting D, Bergfeld W, Miller J, Hordinsky M, Wanser R, et al. A multicenter, randomized, placebo-controlled, double-blind clinical trial of a novel formulation of 5% Minoxidil topical foam versus placebo in the treatment of androgenetic alopecia in men. J Am Acad Dermatol. 2007; 57:767-74).

Scalp hair growth is compared to baseline (Visit X) using the following 7-point scale:
[−3] greatly decreased
[−2] moderately decreased
[−1] slightly decreased
[0] no change
[1] slightly increased
[2] moderately increased
[3] greatly increased
   for the following statement:
“My scalp hair growth compared to Baseline is: . . . ”

### Efficacy Variables: Hair Growth Index (HGI):

Hair Growth Index (HGI) (cf. Gubelin Harcha W, Barboza Martinez J, Tsai T F, Katsuoka K, Kawashima M, Tsuboi R, et al. A randomized, active- and placebo-controlled study of the efficacy and safety of different doses of dutasteride versus placebo and finasteride in the treatment of male subjects with androgenetic alopecia. J Am Acad Dermatol. 2014; 70:489-98 e3)

Scalp hair growth is compared to baseline (Visit X) on a health outcome questionnaire using the following

### 7 Point Scale:

[−3] much less
[−2] moderately less
[−1] slightly less
[0] the same amount
[1] slightly more
[2] moderately more
[3] much more
   for the following 3 questions:
“Since the start of treatment, when I look at my thinning area, I can see . . . (scalp)”.
   “Since the start of treatment, my hair now covers . . . (scalp)”.
   “Since the start of treatment, the appearance (thickness/quality/amount) of the thinning area on my head is . . . ”.

Hair Growth Satisfaction Scale (HGSS) (cf. Gubelin Harcha W, Barboza Martinez J, Tsai T F, Katsuoka K, Kawashima M, Tsuboi R, et al. A randomized, active- and placebo-controlled study of the efficacy and safety of different doses of dutasteride versus placebo and finasteride in the treatment of male subjects with androgenetic alopecia. J Am Acad Dermatol. 2014; 70:489-98 e3)

Scalp hair growth/appearance is compared to baseline (Visit X) using the following 7-point scale:
[−3] very dissatisfied
[−2] dissatisfied
[−1] somewhat dissatisfied
[0] neutral/neither satisfied nor dissatisfied
[1] somewhat satisfied
[2] satisfied
[3] very satisfied
   for 5 questions, “How satisfied do you feel about . . . ”:
The overall appearance of your hair.
   The appearance of the thinning area(s) within treatment areas on your head.
   The amount of scalp that can be seen in the treatment areas.
   The amount of hair in the treatment areas.
   The growth of hair in the treatment areas.

Investigator global assessment (IGA) of scalp hair growth will be performed using the following 7-point scale:
[−3] greatly decreased
[−2] moderately decreased
[−1] slightly decreased
[0] no change
[1] slightly increased
[2] moderately increased
[3] greatly increased

Furthermore, DLQI and Hairdex® (cf. Fischer T W, Schmidt S, Strauss B, Elsner P. Hairdex Ein Instrument zur Untersuchung der krankheitsbezogenen Lebensqualität bei Patienten mit Haarerkrankungen. Hautarzt., 2001 March; 52(3):219-27) can be answered by the subjects.

### Safety Variables:

Medical and gynecological history
Physical examination
Gynecological examination
Breast examination
Dermatological examination of the skin
Vital signs
Electrocardiogram (ECG)
Papanicolaou (PAP) test
Transvaginal ultrasound (TVUS)
Mammography
Laboratory examinations:

### Fasted Blood Sampling for

Hematology/chemistry
   Lipid/glucose parameters for inclusion

### Blood Sampling for:

Follicle-stimulating hormone (FSH) and thyroid-stimulating hormone (TSH) for inclusion
Urine pregnancy test.
Prior and concomitant medication will be checked and observed during the entire study period.
Adverse events (AEs) will be monitored and collected during the entire study period.

### Inclusion Criteria:

Subjects will be allocated to treatment if they meet all of the following inclusion criteria:
1. Signed and dated written informed consent form and any required privacy authorization prior to the initiation of any trial procedure, after the nature of the trial has been explained according to local regulatory requirements;
2. Females, ≥40 up to ≤65 years of age at screening;
3. For hysterectomied subjects: documented hysterectomy must have occurred at least 6 weeks prior to the start of screening. Hysterectomy can be total or subtotal (i.e., cervix was not removed);
4. For non-hysterectomied subjects: uterus with bi-layer endometrial thickness ≤4 mm on TVUS;
5. Seeking treatment for relief of VMS associated with menopause;
6. Body mass index ≥18.5 kg/m² up to ≤35.0 kg/m²;
7. A mammogram that shows no sign of significant disease performed during screening or within 9 months prior to the start of screening¹;
8. Postmenopausal status defined as any of the following:
   a. For non-hysterectomied subjects:
      At least 12 months of spontaneous amenorrhea
      or 6 months of spontaneous amenorrhea with serum FSH levels >40 mlU/mL (values obtained after washout of estrogen/progestin containing drugs, if applicable, see exclusion criteria 18 and 20)
      or 6 weeks postsurgical bilateral oophorectomy2 with or without hysterectomy;
   b. For hysterectomied subjects:
      serum FSH >40 mlU/mL (values obtained after washout of estrogen/progestin containing drug, if applicable, see exclusion criteria 18 and 20);
      or at least 6 weeks postsurgical bilateral oophorectomy²;
9. Good physical and mental health, in the judgement of the Investigator as based on medical history, physical and gynecological examination, and clinical assessments performed prior to Visit 1;
10. Able to understand and comply with the protocol requirements, instructions, and protocol-stated restrictions;
11. Able and willing to complete trial daily diaries and questionnaires.
12. Non-smoker of at least one year (including electronic cigarettes)

Further inclusion criteria considering skin condition:
13. Decrease in hair density on the top of the scalp, relative to the sides and back of the scalp, with scalp hair density in involved density area stages n. 2 to n. 6 on the Savin Density Scale.
14. Subject is willing to maintain the same hairstyle, hair length, and hair color throughout the study (implying the same hair coloring interval prior to the approved photos at screening/baseline).
15. Subject agrees to continue her other general hair care products and regimen for the entire study.
16. Subject agrees to maintain same dietary and supplement pattern.

¹Subjects must have a Breast Imaging-Reporting And Data System (BI-RADS) score of 1 or 2 to enroll in the study. An incomplete mammogram result, i.e., BI-RADS 0, is not acceptable and requires further assessment. The site must obtain a copy of the official report for the subject's study file. A digitalized imaging should be obtained if mammography is done as part of this study.

²A report or a statement on letterhead from the subject's physician documenting both ovaries were removed is needed.

### Exclusion Criteria:

Subjects will not be allocated to treatment if they meet one of the following exclusion criteria:
1. History of malignancy, with the exception of basal cell or non-metastatic squamous cell carcinoma of the skin if diagnosed more than 1 year prior to the screening visit³;
2. Any clinically significant findings found by the Investigator at the breast examination and/or on mammography suspicious of breast malignancy that would require additional clinical testing to rule out breast cancer (however, simple cysts confirmed by ultrasound are allowed);
3. PAP test with atypical squamous cells undetermined significance (ASC-US) or higher (low-grade squamous intraepithelial lesion [LSIL], atypical squamous cells cannot exclude high-grade squamous intraepithelial lesion [HSIL] [ASC-H], HSIL dysplastic or malignant cells) in sub-totally hysterectomied and non-hysterectomied subjects⁴. Note: ASC-US is allowed if a reflex human papilloma virus (HPV) testing is performed and is negative for high risk oncogene HPV subtype 16 and 18;
4. For non-hysterectomied subjects:
   a. Presence of uterine cancer, endometrial hyperplasia;
   b. Presence of endometrial polyp(s);
   c. Undiagnosed vaginal bleeding or undiagnosed abnormal uterine bleeding
   d. Endometrial ablation;
   e. Any uterine/endometrial abnormality that in the judgment of the investigator contraindicates the use of estrogen and/or progestin therapy. This includes presence or history of adenomyosis or significant myoma;
5. Systolic blood pressure (BP) higher than 139 mmHg, diastolic BP higher than 89 mmHg⁵ during screening;
6. History of venous or arterial thromboembolic disease (e.g., superficial or deep vein thrombosis, pulmonary embolism, stroke, myocardial infarction, angina pectoris, etc.), or first-degree family history of venous thromboembolism (VTE);
7. History of known acquired of congenital coagulopathy or abnormal coagulation factors, including known thrombophilia's;
8. Laboratory values of fasting glucose above 125 mg/dL and/or glycated hemoglobin above 7.5% 6;
9. Dyslipoproteinemia (LDL >190 mg/dL and/or triglycerides >300 mg/dL) 7;
10. Presence or history of gallbladder disease, unless cholecystectomy has been performed;
11. Systemic lupus erythematosus;
12. Any malabsorption disorders including gastric bypass surgery;
13. History of acute liver disease in the preceding 12 months before the start of screening or presence or history of chronic or severe liver disease [alanine transaminase (ALT) or aspartate transaminase (AST)>2× upper limit of normal (ULN), bilirubin >1.5 ULN], or liver tumors;
14. Chronic or current acute renal impairment (estimated glomerular filtration rate <60 mL/min);
15. Positive human immunodeficiency virus (HIV), hepatitis B or C serology;
16. Porphyria;
17. Diagnosis or treatment of major psychiatric disorder (e.g., schizophrenia, bipolar disorder, etc.) in the judgement of the Investigator;
18. Use of estrogen/progestin containing drug(s) up to:
   a. 1 week before screening start for vaginal non-systemic hormonal products (rings, creams, gels);
   b. 4 weeks before screening start for vaginal or transdermal estrogen or estrogen/progestin products with systemic effect;
   c. 8 weeks before screening start for oral estrogen and/or progestin products and/or selective estrogen receptor modulator therapy;
   d. 8 weeks before screening start for intrauterine progestin therapy;
   e. 3 months before screening start for progestin implants or estrogen alone injectable drug therapy;
   f. 6 months before screening start for estrogen pellet therapy or progestin injectable drug therapy;
19. Use of androgen/dehydroepiandrosterone (DHEA) containing drugs:
   a. 8 weeks before screening start for oral, topical, vaginal or transdermal androgen;
   b. 6 months before screening start for implantable or injectable androgen therapy;
20. Use of phytoestrogens or black cohosh for treatment of VMS up to 2 weeks before the start of screening;
21. Not willing to stop any hormonal products as described in exclusion criteria 18, 19 and 20 during their participation in the trial;
22. Inadequately treated thyroid dysfunction. Subjects with low or high TSH are allowed if free thyroxine (T4) at screening is within normal range⁸.
23. History or presence of allergy/intolerance to the investigational product or drugs of this class or any component of it, or history of drug or other allergy that, in the opinion of the Investigator contraindicates subject participation;
24. History of alcohol or substance abuse (including marijuana, even if legally allowed) or dependence in the previous 12 months before the start of screening as determined by the Investigator, based on reported observations;
25. Sponsor or CRO employees or employees under the direct supervision of the Investigator and/or involved directly in the trial;
26. Subjects with known or suspected history of a clinically significant systemic disease, unstable medical disorders, life-threatening disease or current malignancies that would pose a risk to the subject in the opinion of the Investigator;
27. Participation in another investigational drug clinical trial within 1 month (30 days) or having received an investigational drug within the last month (30 days) before the start of screening;
28. Is judged by the Investigator to be unsuitable for any reason.

Further exclusion criteria considering impact on hair condition:
29. Subject has any dermatological disorders of the scalp in the target region with the possibility of interfering with the application of the IMP or examination method, such as fungal or bacterial infections, seborrheic dermatitis, psoriasis, eczema, folliculitis, scars, or scalp atrophy.
30. Subject has any skin pathology or condition that, in the investigator's opinion, could interfere with the evaluation of the IMP or requires use of interfering topical, systemic (e.g., uncontrolled thyroid disease, certain genetic disorders that involve hair growth or patterns), or surgical therapy.
31. Subject has a history or any signs of hyperandrogenemia (e.g., excessive facial/pubic/periumbilical hair growth, severe acne, excessive testosterone values in medical history).
32. Subject has current or recent history (within 6 months) of hair weaves, non-breathable wigs or hair bonding.
33. Subject had scalp hair transplants at any time.
34. Subject has a history or active hair loss due to diffuse telogen effluvium, alopecia areata, scarring alopecia, trichotillomania, or conditions/diseases other than AGA.
35. Subject has a current or recent history (within 6 months) of severe dietary or weight changes or has a history of eating disorder(s), any history of bariatric surgery (gastric bypass, gastric sleeve, stomach stapling); macro- or micro-nutrient deficiencies within the last 6 months (i.e., clinically significant iron deficiency, protein deficiency confirmed by laboratory testing) and/or any current diagnosis of malabsorptive disease (i.e., Celiac, Irritable Bowel disease etc.).
36. Subject has used any of the following topical preparations or procedures on the scalp:
   a) Topical scalp treatments for hair growth including Minoxidil, hormone therapy, anti-androgens, or other agents that are known to affect hair growth within 12 weeks prior to baseline (Visit 2).
   b) Topical scalp over-the-counter (OTC) or cosmetic treatments known or reasonably believed to affect hair growth (e.g., brands such as Maxilene, Nioxin, Foltene, etc.) or hair health or hair growth products with saw palmetto, copper, etc. within 4 weeks prior to baseline (Visit 2).
   c) Topical scalp treatments that may have ancillary effect on hair growth including, but not limited to, corticosteroids, pimecrolimus, tacrolimus, and retinoids within 4 weeks prior to baseline (Visit 2).
   d) Scalp procedures (surgical, laser, light, or energy treatments, micro-needling, etc.) within 6 months prior to baseline (Visit 2).
   e) Platelet rich plasma (PRP) procedure on the scalp within 1 year.
37. Subject has used the following systemic medications or procedures:
   a) Beta blockers, cimetidine, diazoxide, or corticosteroids (including intramuscular and intralesional injections) within 12 weeks of Visit 2/baseline. Inhaled, intranasal, or ocular corticosteroids are allowed if use is stable [defined as doses and frequency unchanged for at least 4 weeks prior to baseline (Visit 2)].
   b) Retinoid, isotretinoin, vitamin A intake above 10,000 IU per day, or cyclosporine therapy within 6 months prior to baseline (Visit 2).
   c) Any 5 alpha reductase medications (i.e., Finasteride (Propecia®, etc.), Dutasteride or similar product(s)) within 12 months prior to baseline (Visit 2).
   d) Chemotherapy or cytotoxic agents in the last 5 years.
   e) Radiation of the scalp at any time point.
   f) Anti-androgens within 6 months prior to baseline (Visit 2); Anti-androgens (cyproterone acetate, chlormadinone acetate, etc.) together with contraceptive pills are allowed if use is stable [defined as doses and frequency unchanged for at least 3 months prior to baseline (Visit 2)].
   g) Other systemic therapy, which in the opinion of the investigator, may materially affect the subject's hair or hair growth, including, but not limited to, spironolactone, vitamin (including biotin intake >5 mg/day) or homeopathy supplement hair growth or hair health products or other steroid hormones (in any form), including anabolic steroids during the 3 months prior to baseline (Visit 2) or during the study.

³ The exception is only for basal cell carcinoma or non-metastatic squamous cell carcinoma of the skin if either of them was diagnosed more than one year prior to the screening of the subject.

⁴ As indicated by written documentation of a prior test performed within 18 months prior screening or by a test performed at screening.

⁵ BP measurements at screening may be repeated if values are outside the inclusion criteria after sitting for an additional 5 to 10 minutes. The last reading will be used for eligibility. Subjects with mild to moderate hypertension who are controlled on a stable antihypertension regimen may be enrolled if they meet all inclusion/exclusion criteria. Subjects using methyldopa or clonidine containing antihypertensive medication will not be included.

⁶ Laboratory values of fasting glucose and glycated hemoglobin assessed during the last 6 months, and during washout and screening should be considered.

⁷ Subjects using lipid-lowering therapy should be on a stable dose for at least 1 month before screening.

⁸ Reflex T4 test to be performed at screening only if TSH at screening is outside normal range.

### Statistical Methods:

Analyses will be performed on the intention-to-treat and per-protocol population. Categorical factors will be summarized using frequencies and/or percentages, while continuous measures will be described using means and standard deviations.

For normally distributed data, the significance of the within-group change from baseline will be estimated from the Student's t distribution and between-group comparison of the change from baseline will be performed using independent t-tests. Results will be presented as the mean and 95% confidence interval. Equivalent nonparametric methods will be used for non-normally distributed data: Wilcoxon sign test for the within-group change from baseline and Mann-Whitney U test for the between-group comparison. Results will be presented as the median, minimum and maximum.

Analyses will be performed using SAS software (Stat version 15.2; SAS Institute, Cary, NC, USA) with a significance level of 0.05. In this exploratory POC trial, no adjustment for multiplicity of testing will be performed.

### Example 12: Randomized Double-Blinded, Placebo-Controlled Study Evaluating the Effects of Estetrol (E4) Topical Formulation on Hair Parameters in Men with Excessive Hair Shedding

Test product: Estetrol monohydrate topical formulation 0.06%, 0.22% or 0.50% respectively formulated as a solution using ethanol, propylene glycol, and water. Note that due to solubilisation during the formulation process the estetrol monohydrate may no longer be present in the monohydrate form, which should be evaluated for each topical composition separately.

Control product: placebo formulation

All study treatment will be given by the study investigator or designated member of staff to patients for self-administration. The four formulations will be provided in identical bottle with a number from 1 to 120. Patients will be asked to apply product topically on dry hair and scalp, preferably before sleeping, once a day, by massaging softly the scalp, for the entire trial period of 24 weeks.

### Objectives

### Primary Objective:

To assess the hair growth-promoting capacity of the E4 topical formulation in comparison to a placebo formulation, in men with clinical signs of excessive hair shedding by investigating: Hair shedding, Hair volume, Hair thickness, Hair density, Hair miniaturization, Anagen prolongation, Patient Self-Assessment.

### Secondary Objective:

To assess the skin compatibility and tolerability to the E4 topical formulation in comparison to a placebo formulation in men with clinical signs of excessive hair shedding by investigating: Patient Self-Assessment, and Clinical Observation of Adverse Effects.

### Methodology

120 healthy male volunteers from 18 to 65 years old complaining of hair shedding of more than 4 weeks duration will be asked to apply E4 topical formulation or placebo formulation to their scalp for the entire trial period of 24 weeks. Each patient will be randomly allocated to either of the four groups (n=30 in each group): three treatment group receiving E4 topical formulation at different strengths and a placebo group. Patients will visit the clinic at week 0, week 8, and week 24.
Number of patients: 120, 30 per study arm
Duration of treatment: 24 weeks

### Diagnosis and Main Criteria for Inclusion

Patients with confirmed diagnosis of male androgenetic alopecia and who received no administration of topical or systemic therapies for hair loss during the two months (including oral nutritional supplements) prior the initiation of the study will be included in the trial.

### Criteria for Evaluation

### Primary:

### Hair Growth Assessment:

Hair Shedding Visual Scale: After two non-wash days, patients will be asked to wash, comb and collect all hairs into a plastic bag before visitation to the clinic. The collected hairs will be subsequently evaluated using an arbitrary unit from 1 to 9 by following the Hair Shedding Visual Scale, described by Sinclair and recently validated by Tosti et al. (Tosti et al., Dermatol Ther, 2017).
Cross-Sectioned Trichometer (Hair Check®): The cross-sectional area of a bundle of hair selected from a specific 2 cm×2 cm area located in the mid-parietal area of the scalp, using a measuring template (numbered at 10 every time), which corresponds to approximately 12.5 cm from the glabella. It will be evaluated using the Hair Check® device. The quantitative result, known as the hair mass index, is an indirect measurement of hair volume.
Hair Thickness (Folliscope®): The hair diameter of 10-15 hairs will be measured with the Folliscope® device, at the same area of the scalp as the hair check. The mean of hair diameter will be calculated.
Terminal/Vellus Hair Ratio: Based on the measurement of hair thickness using the Folliscope® device, the number of terminal (defined as hairs >40 microns) and vellus or miniaturized hairs (defined as hairs <40 microns) will be counted.
Hair density: The number of hair/cm² will be measured using the device TrichoScan® after shaving a 1.6 cm diameter circle into the scalp and dyeing this area. Hair density will be measured within a 0.6 cm² area adjacent to the right side of the area used for hair check.
Phototrichogram (Anagen/Catagen-Telogen Ratio): The same area used for hair density will be used to determine the anagen/catagen-telogen ratio using digital video microscopy of the TrichoScan®.

The number of growing (anagen) hairs will be counted manually 48 hours after shaving. Hairs that do not grow after 48 hours will be regarded as catagen/telogen.

Patient Self-Assessment Questionnaire: A self-assessment questionnaire containing the following questions will be given to all subject at day 60 and day 180: P1: Visible decrease in hair shedding, P2: I find less hairs in the comb/brush, P3: It seems that my hairs are growing, P4: My hairs are more resistant after stress, P5: The quality of my hairs is better, P6: My hairs are stronger, P7: My hairs are denser, P8: My hairs are thicker, P9: My hairs are soft, P10: My hairs are shiny, P11: My hairs have increased volume, P12: I am satisfied with the results.

### Secondary:

Patient Self-Assessment Questionnaire: A self-assessment questionnaire containing the following questions will be given to all subject at day 60 and day 180: P13: Visual impression, P14: Smell impression, P15: Tolerability to product.
Clinical Observation of Adverse effect, i.e. redness and scaling of scalp skin

### Predicted Conclusions

We predict that E4 topical formulation will ameliorate clinical signs of male androgenetic alopecia in a dose-dependent manner and more efficiently than placebo formulation and protect against HF miniaturization (i.e. vellus-to-terminal hair reconversion).

Most of the effects will already be seen after 8 weeks of treatment and will be maintained at week 24.

### Example 13. Preparation of Aqueous Gel Formulations for Stability Studies

Table 8 lists the hydrogels, that were prepared for stability studies. Preparation took place as described for AG24 in Example 1.

TABLE 8
Compositions (% w/w) of estetrol (E4) aqueous gel
formulations prepared for stability studies.

| Formulation | 1 | 2 | 3 | 4 | 5 |
|---|---|---|---|---|---|
| Estetrol (E4) | 0.06 | 0.40 | 0.50 | 0.06 | 0.50 |
| PEG 400 | 43.95 | 43.95 | 43.95 | 43.95 | 43.95 |
| Transcutol ® P | 1.00 | 1.00 | 1.00 | 2.50 | 2.50 |
| Water (1st Addition) | 48 | 48 | 48 | 48 | 48 |
| Carbopol ® 980 | 0.50 | 0.50 | 0.50 | 0.50 | 0.50 |

| | |
|---|---|
| Sodium hydroxide | Adjust to pH 6-6.5 |
| Water (2^{nd} addition) | q.s. 100% |
| TOTAL | 100 |

1-15. (canceled)

16. A method of preventing or treating hair loss in a subject in need thereof, comprising topically applying to the subject's scalp a topical composition comprising an effective amount of an estetrol component.

17. The method according to claim 16, wherein the subject is a male subject.

18. The method according to claim 16, wherein the composition comprises from about 0.01% (w/w) to about 5% (w/w) of the estetrol component.

19. The method according to claim 16, wherein the composition comprises from about 0.02% (w/w) to about 1% (w/w) of the estetrol component.

20. The method according to claim 16, wherein the composition comprises from 0.1% (w/w) to 1% (w/w) of the estetrol component.

21. The method according to claim 16, wherein the composition comprises from 0.3% (w/w) to 0.7% (w/w) of the estetrol component.

22. The method according to claim 16, wherein the composition further comprises a permeation enhancer.

23. The method according to claim 16, wherein the composition is in a form selected from solutions, emulsions, suspensions, ointments, pastes, lotions, gels, foams, mousses, creams, and sprays.

24. The method according to claim 16, wherein the estetrol component is estetrol monohydrate.

25. The method according to claim 16, wherein the topical application of the topical composition leads to an improvement in or maintenance of one or more selected from hair texture, hair quality, and hair appearance.

26. The method according to claim 16, wherein the topical application of the topical composition leads to an improvement in one or both of target area hair count (TAHC) and target area hair width (TAHW).

27. The method according to claim 16, wherein the topical application of the topical composition is effective for one or more of preventing hair loss, reversing hair loss, slowing down hair loss, reducing hair loss, and increasing hair growth.

28. The method according to claim 16, wherein said hair loss is selected from female pattern hair loss (female androgenetic alopecia (FAGA)) and male pattern hair loss (MAGA).

29. The method according to claim 16, wherein the topical application of the topical composition is effective for one or more of increasing function of keratinocytes and increasing growth of keratinocytes.

30. The method according to claim 29, wherein the keratinocytes are epidermal keratinocytes (KCs) residing in a bulge, outer root sheet (ORS), or hair bulb.

31. The method according to claim 16, wherein the topical application of the topical composition is effective to increase one or more of hair follicle mesenchymal fibroblast function, hair follicle mesenchymal fibroblast growth, dermal papilla (DP) cell function, and dermal papilla (DP) cell growth.

32. The method according to claim 16, wherein the topical application of the topical composition is effective to delay onset of catagen or prevent catagen.

33. The method according to claim 16, wherein the topical application of the topical composition is effective to promote anagen or prolong anagen.

34. The method according to claim 16, wherein the topical application of the topical composition is effective to restore or promote epithelial-mesenchymal interaction between hair follicle cells.

35. The method according to claim 16, wherein the topical application of the topical composition is effective to reduce one or both of inflammation and oxidative stress in hair follicles.
